(19) United States
(12) Patent Application Publication
(10) Pub. No.: US 2023/0223105 A1
(43) Pub. Date: Jul. 13, 2023

(54) MITIGATION OF STATISTICAL BIAS IN GENETIC SAMPLING

(71) Applicant: Foundation Medicine, Inc., Cambridge, MA (US)

(72) Inventors: Lee Alan ALBACKER, Arlington, MA (US); Meagan Kathleen MONTESION, Somerville, MA (US)

(73) Assignee: Foundation Medicine, Inc., Cambridge, MA (US)

(21) Appl. No.: 17/802,436

(22) PCT Filed: Feb. 26, 2021
(86) PCT No.: PCT/US2021/019982

§ 371 Date: Aug. 25, 2022

Related U.S. Application Data
(60) Provisional application No. 63/093,015, filed on Oct. 16, 2020.
(60) Provisional application No. 62/982,677, filed on Feb. 27, 2020.

(51) Int. Cl. G16B 20/20; C12Q 1/6886; G16B 40/00

(52) U.S. Cl. CPC G16B 20/20; C12Q 1/6886; G16B 40/00; C12Q 2600/156

(57) ABSTRACT

Provided herein are methods, systems, and storage media that may find use, e.g., in sequencing (e.g., via NGS) polymorphic alleles, such as detecting loss-of-heterozygosity (LOH) of a human leukocyte antigen (HLA) gene or another polymorphic human gene. In some embodiments, the methods and systems comprise obtaining observed allele frequencies and observed binding propensities for the alleles to one or more bait molecule(s), then applying an optimization model to determine adjusted allele frequencies that take into account these binding propensities, thereby adjusting for and/or minimizing any potential bias rooted in differential allele: bait binding propensities with regard to determination of allele frequency.

### CROSS-REFERENCE TO RELATED APPLICATIONS

This application claims the benefit of U.S. Provisional Application Nos. 62/982,677, filed Feb. 27, 2020, and 63/093,015, filed Oct. 16, 2020, each of which is hereby incorporated by reference in its entirety.

### TECHNICAL FIELD

This application relates to mitigating sampling bias in genomic tests that use a hybrid capture process.

### BACKGROUND

Genomic testing has the potential to identify patients that are more likely to respond to certain treatments. Some genomic testing approaches use a hybrid capture step, e.g., to enrich for sequence reads at certain loci of interest (Frampton, G. M. et al. (2013) Nat. Biotechnol. 31:1023-1031). However, when hybrid capture is applied to polymorphic alleles, differences in allelic binding to specific capture probes have the potential to introduce bias into sampling. Accurate genomic profiling requires unbiased sequencing and counting of allele frequency regardless of specific polymorphisms.

One area in which genomic testing has been applied is determining personalized genomic information for cancer treatment. Highly polymorphic alleles may present challenges to obtaining accurate and comprehensive genomic information. For instance, some tumors are characterized by loss of HLA-I allele(s) (termed loss-of-heterozygosity or LOH). Whether or not a tumor has experienced LOH at certain genetic locations can be an important clinical fact, especially when the genetic location is relevant to important biological functions such as the person's immune system or other functions. For example, LOH at one or more HLA-I alleles can result in fewer neoantigens being presented to the immune system, leading to immune escape of the tumor. In addition, various loci can be modified by copy-loss LOH (i.e., loss of one allele) or by copy-neutral LOH (i.e., where one allele is lost but the other is duplicated, resulting in no net change in copy number).

Immunotherapies have revolutionized current treatments for advanced cancer patients. Some (e.g., cell-based therapies) provide or stimulate an immune response to the cancer, while others (e.g., immune checkpoint inhibitors or ICIs) are thought to reinvigorate the patient's own T-cell mediated immune response [Reck, M., et al. N Engl J Med 375, 1823-1833 (2016); Hellmann, M. D., et al. N Engl J Med 378, 2093-2104 (2018); Nghiem, P. T., et al. N Engl J Med 374, 2542-2552 (2016); Robert, C., et al. N Engl J Med 372, 2521-2532 (2015); Le, D. T., et al. N Engl J Med 372, 2509-2520 (2015)]. The adaptive immune system, via CD8+ T cells, recognizes tumor cells via the presentation of tumor-specific mutant peptides (neoantigens) presented on human leukocyte antigen class I (HLA-I)-encoded major histocompatibility complex class I (MHC-I) proteins [Mok, T. S. K., et al. Lancet 393, 1819-1830 (2019); Schumacher, T. N. & Schreiber, R. D. Science 348, 69-74 (2015); Turajlic, S., el al. Lancet Oncol 18, 1009-1021 (2017)]. From this perspective, it seems intuitive that tumors with increased tumor mutational burden (TMB) would be more likely to be targeted by immune stimulation via ICIs due to a greater number of potential neoantigens available for presentation [Hellmann, M. D., et al. N Engl J Med 378, 2093-2104 (2018); Le, D. T., et al. N Engl J Med 372, 2509-2520 (2015); Rizvi, N. A., et al. Science 348, 124-128 (2015)], but this may not always be the case. For example, in trials focused on non-small cell lung cancer (NSCLC), TMB failed to sufficiently predict for patient survival. However, efforts to use HLA genotyping to predict the relative efficiency of neoantigen presentation and use of this information together with TMB to predict checkpoint responses are showing promise (Goodman A M, et al. Genome Med. 2020; 12(1):45; Shim J H, et al. Ann Oncol. 2020; 31(7).902-11).

Response to immunotherapies such as ICI treatment have been found to be variable among different patients. In order to ensure that each patient receives a treatment that is most likely to be effective for their particular tumor, further methods and systems are needed to obtain unbiased polymorphic allele sequence and frequency, e.g., to predict responsiveness to immunotherapies and quickly stratify patients for the most potentially efficacious treatment.

### SUMMARY OF THE INVENTION

Accordingly, provided herein are methods and systems for mitigating sampling bias introduced via hybrid capture. These methods and systems account for and mitigate against biases that can be introduced when obtaining genomic data related to polymorphic alleles, e.g., resulting from hybrid capture of polynucleotides for sequencing.

For example, one highly polymorphic locus of the human genome that is critical for personalized treatment approaches is the HLA-I locus. Stratifying potential immunotherapy patients using LOH at the HLA-I locus has the potential to identify patients most likely to respond to immune-reinvigorating treatments such as ICIs. As demonstrated herein, somatic loss of HLA-I was shown to be a negative predictor of patient survival in ICI-treated NSCLC, which blunts the effect of high TMB. The landscape of somatic HLA-I LOH in over 83,000 patient samples across 59 disease groups was also determined, finding a pan-cancer incidence of 17% and significant enrichment in tumors with high TMB and inflamed tumors as represented by PD-L1 expression. Combined, TMB and HLA-I LOH may better select patients most likely to benefit from ICI in inflamed cancers and has implications for the design of personalized cancer vaccines. Other genetic loci known to be involved in LOH events are also described herein.

Described herein is a method, comprising identifying a plurality of chemical reactions such that: each reaction corresponds to a bait molecule binding to a different allele of a polymorphic gene, and each reaction resulting in the capture of a corresponding allele fraction; and the plurality of chemical reactions consists of a first subset of reactions and a second subset of reactions, in which the first and second subsets share no reaction in common and in which the first and second subsets each comprise at least one chemical reaction; identifying a plurality of equations that collectively relate binding propensities of each chemical reaction and allele fraction of each captured allele; empirically identifying the relative binding propensities of the first subset of the plurality of chemical reactions; and identifying the relative binding propensities of the second subset by minimizing a total error.

In some embodiments, minimizing the total error is subject to the constraint that the median relative binding propensities is equal to 1.

In some embodiments, one relative binding propensity is set equal to 1.

In some embodiments, minimizing the total error includes performing a least squares procedure.

In some embodiments, the method further comprises performing a hybrid capture process to measure raw allele frequencies in a DNA sample of a patient; and using the first and second subsets of relative binding propensities to scale the measured raw allele frequencies, thereby mitigating sampling bias.

In some embodiments, the polymorphic gene includes a Human Leukocyte Antigen gene. In some embodiments, the polymorphic gene is ST7/RAY1, ARH1/NOEY2, TSLC1, RB, PTEN, SMAD2, SMAD4, DCC, TP53, ATM, miR-15a, miR-16-1, NAT2, BRCA1, BRCA2, hOGG1, CDH1, IGF2, CDKN1C/P57, MEN1, PRKAR1A, H19, KRAS, BAP1, PTCH1, SMO, SUFU, NOTCH1, PPP6C, LATS1, CASP8, PTPN14, ARID1A, FBXW7, M6P/IGF2R, IFN-alpha, an olfactory receptor gene, CBFA2T3, DUTT1, FHIT, APC, P16, FCMD, TSC2, miR-34, c-MPL, RUNX3, DIRAS3, NRAS, miR-9, FAM50B, PLAGL1, ER, FLT3, ZDBF2, GPR1, c-KIT, NAP1L5, GRB10, EGFR, PEG10, BRAF, MEST, JAK2, DAPK1, LIT1, WT1, NF-1, PR, c-CBL, DLK1, AKT1, SNURF, a cytochrome P450 gene (CYP), ZNF587, SOCS1, TIMP2, RUNX1, AR, CEBPA, C19MC, EMP3, ZNF331, CDKN2A, PEG3, NNAT, GNAS, or GATA5.

In some embodiments, the method further comprises determining whether the patient has experienced a loss of heterozygosity.

Also described herein is a system, comprising: one or more processors; and a memory configured to store one or more computer program instructions, wherein the one or more computer program instructions when executed by the one or more processors are configured to: identify a plurality of chemical reactions such that: each reaction corresponds to a bait molecule binding to a different allele of a polymorphic gene, and each reaction resulting in the capture of a corresponding allele fraction; and the plurality of chemical reactions consists of a first subset of reactions and a second subset of reactions, in which the first and second subsets share no reaction in common and in which the first and second subsets each comprise at least one chemical reaction; identify a plurality of equations that collectively relate binding propensities of each chemical reaction and allele fraction of each captured allele; receive empirically identified relative binding propensities of the first subset of the plurality of chemical reactions; and identify the relative binding propensities of the second subset by minimizing a total error.

In some embodiments of the system, minimizing the total error is subject to the constraint that the median relative binding propensities is equal to 1.

In some embodiments of the system, one relative binding propensity is set equal to 1.

In some embodiments of the system, minimizing the total error includes performing a least squares procedure.

In some embodiments of the system, the method further comprises: receiving, at the one or more processors, measured raw allele frequencies in a DNA sample of a patient, wherein the measured raw allele frequencies were measured by performing a hybrid capture process; and scaling, at the one or more processors, the measured raw allele frequencies using the first and second subsets of relative binding propensities, thereby mitigating sampling bias.

In some embodiments of the system, the polymorphic gene includes a Human Leukocyte Antigen gene. In some embodiments of the system, the polymorphic gene is ST7/RAY1, ARH1/NOEY2, TSLC1, RB, PTEN, SMAD2, SMAD4, DCC, TP53, ATM, miR-15a, miR-16-1, NAT2, BRCA1, BRCA2, hOGG1, CDH1, IGF2, CDKN1C/P57, MEN1, PRKAR1A, H19, KRAS, BAP1, PTCH1, SMO, SUFU, NOTCH1, PPP6C, LATS1, CASP8, PTPN14, ARID1A, FBXW7, M6P/TGF2R, IFN-alpha, an olfactory receptor gene, CBFA2T3, DUTT1, FHIT, APC, P16, FCMD, TSC2, miR-34, c-MPL, RUNX3, DIRAS3, NRAS, miR-9, FAM50B, PLAGL1, ER, FLT3, ZDBF2, GPR1, c-KIT, NAP1L5, GRB10, EGFR, PEG10, BRAF, MEST, JAK2, DAPK1, LIT1, WT1, NF-1, PR, c-CBL, DLK1, AKT1, SNURF, a cytochrome P450 gene (CYP), ZNF587, SOCS1, TIMP2, RUNX1, AR, CEBPA, C19MC, EMP3, ZNF331, CDKN2A, PEG3, NNAT, GNAS, or GATA5.

In some embodiments of the system, the method further comprises determining, at the one or more processors, whether the patient has experienced a loss of heterozygosity.

Certain aspects of the present disclosure relate to methods for determining allele frequency. In some embodiments, the methods comprise: a) receiving, at one or more processors, an observed allele frequency for an allele of a gene, wherein the observed allele frequency corresponds to frequency of nucleic acid(s) encoding at least a portion of the allele as detected among a plurality of sequence reads corresponding to the gene, wherein the plurality of sequence reads was obtained by sequencing nucleic acids encoding the gene or a portion thereof as captured by hybridization with a bait molecule; b) receiving, at one or more processors, a relative binding propensity for the allele to the bait molecule, wherein the relative binding propensity of the allele corresponds to propensity of nucleic acid encoding at least a portion of the allele to bind the bait molecule in the presence of nucleic acids encoding portions of one or more other alleles of the gene; c) executing, by the one or more processors, an objective function to measure a difference between the relative binding propensity and the observed allele frequency of the allele; d) executing, by the one or more processors, an optimization model to minimize the objective function; and e) determining, by the one or more processors, an adjusted allele frequency of the allele based on the optimization model and the observed allele frequency.

In some embodiments, the optimization model is a least squares optimization model. In some embodiments, the optimization model is subject to one or more constraints. In some embodiments, the one or more constraints require that median value of the relative binding propensities for a plurality of alleles of the gene is equal to 1. In some embodiments, the observed allele frequency corresponds to relative frequency of nucleic acid(s) encoding at least a portion of the allele as detected among the plurality of sequence reads, as compared to a reference value. In some embodiments, the reference value is a total number of sequence reads. In some embodiments, the reference value is a number of sequence reads corresponding to a reference gene.

In some embodiments according to any of the embodiments described herein, the gene is a human leukocyte antigen (HLA) gene encoding a major histocompatibility (MHC) class I molecule. In some embodiments, the gene is ST7/RAY1, ARH1/NOEY2, TSLC1, RB, PTEN, SMAD2, SMAD4, DCC, TP53, ATM, miR-15a, miR-16-1, NAT2, BRCA1, BRCA2, hOGG1, CDH1, IGF2, CDKN1C/P57, MEN1, PRKAR1A, H19, KRAS, BAP1, PTCH1, SMO, SUFU, NOTCH1, PPP6C, LATS1, CASP8, PTPN14, ARID1A, FBXW7, M6P/IGF2R, IFN-alpha, an olfactory receptor gene, CBFA2T3, DUTT1, FHIT, APC, P16, FCMD, TSC2, miR-34, c-MPL, RUNX3, DIRAS3, NRAS, miR-9, FAM50B, PLAGL1, ER, FLT3, ZDBF2, GPR1, c-KIT, NAP1L5, GRB10, EGFR, PEG10, BRAF, MEST, JAK2, DAPK1, LIT1, WT1, NF-1, PR, c-CBL, DLK1, AKT1, SNURF, a cytochrome P450 gene (CYP), ZNF587, SOCS1, TIMP2, RUNX1, AR, CEBPA, C19MC, EMP3, ZNF331, CDKN2A, PEG3, NNAT, GNAS, or GATA5. In some embodiments, the methods further comprise, after determining the adjusted allele frequency: determining that the gene has undergone loss-of-heterozygosity (LOH) based at least in part on the adjusted allele frequency. In some embodiments, the plurality of sequence reads was obtained by performing next-generation sequencing (NGS), whole exome sequencing, or methylation sequencing on nucleic acids captured by hybridization with the bait molecule. In some embodiments, the methods further comprise, prior to obtaining the observed allele frequency: sequencing a plurality of polynucleotides by next-generation sequencing (NGS), whole exome sequencing, or methylation sequencing in order to obtain the plurality of sequence reads, wherein the plurality of polynucleotides comprises nucleic acid(s) encoding at least a portion of the allele. In some embodiments, the methods further comprise, prior to sequencing the plurality of polynucleotides: contacting a mixture of polynucleotides with the bait molecule under conditions suitable for hybridization, wherein the mixture comprises a plurality of polynucleotides capable of hybridization with the bait molecule; and isolating a plurality of polynucleotides that hybridized with the bait molecule, wherein the isolated plurality of polynucleotides that hybridized with the bait molecule are sequenced. In some embodiments, the methods further comprise, prior to contacting the mixture of polynucleotides with the bait molecule: obtaining a sample from an individual, wherein the sample comprises tumor cells and/or tumor nucleic acids; and extracting the mixture of polynucleotides from the sample, wherein the mixture of polynucleotides is from the tumor cells and/or tumor nucleic acids. In some embodiments, the sample further comprises non-tumor cells. In some embodiments, the sample comprises fluid, cells, or tissue. In some embodiments, the sample comprises blood or plasma. In some embodiments, the sample comprises a tumor biopsy or a circulating tumor cell. In some embodiments, the sample from the individual is a nucleic acid sample. In some embodiments, the nucleic acid sample comprises mRNA, genomic DNA, circulating tumor DNA, cell-free DNA, or cell-free RNA. In some embodiments, the methods further comprise obtaining an observed allele frequency for each of two or more alleles of a gene, wherein the observed allele frequencies correspond to frequency of nucleic acid(s) encoding at least a portion of the respective allele as detected among a plurality of sequence reads corresponding to the gene, wherein the plurality of sequence reads was obtained by sequencing nucleic acids encoding the gene or a portion thereof as captured by hybridization with a bait molecule; obtaining a relative binding propensity for each of two or more alleles to the bait molecule, wherein a second of the two or more alleles has a lower relative binding propensity to the bait molecule than a first of the two or more alleles; and identifying a second bait molecule, wherein the second of the two or more alleles has a higher relative binding propensity to the second bait molecule than to the first bait molecule. In some embodiments, the second bait molecule comprises a sequence complementary to at least a portion of the second of the two or more alleles.

In yet some other aspects, provided herein are methods of selecting a bait molecule, comprising: obtaining an observed allele frequency for two or more alleles of a gene, wherein the observed allele frequencies correspond to frequency of nucleic acid(s) encoding at least a portion of the respective allele as detected among a plurality of sequence reads corresponding to the gene, and wherein the plurality of sequence reads was obtained by sequencing nucleic acids encoding the gene or a portion thereof as captured by hybridization with a first bait molecule; obtaining a relative binding propensity for two or more alleles of a gene to the first bait molecule, wherein a second of the two or more alleles has a lower relative binding propensity to the first bait molecule than a first of the two or more alleles; and identifying or selecting the sequence of a second bait molecule, wherein the second of the two or more alleles has a higher relative binding propensity to the second bait molecule than to the first bait molecule. In some embodiments, the second bait molecule comprises a sequence complementary to at least a portion of the second of the two or more alleles of the gene. In some embodiments, the second bait molecule comprises a sequence based at least in part on the sequences of the second and a third of the two or more alleles of the gene, wherein the second and third alleles have a lower relative binding propensity to the first bait molecule than the first allele.

In yet some other aspects, provided herein are non-transitory computer-readable storage media. In some embodiments, the non-transitory computer-readable storage media comprise one or more programs for execution by one or more processors of a device, the one or more programs including instructions which, when executed by the one or more processors, cause the device to perform the method according to any of the embodiments described herein.

In still some other aspects, provided herein are methods for detecting loss-of-heterozygosity (LOH) of a human leukocyte antigen (HLA) gene. In some embodiments, the methods comprise: a) receiving, at one or more processors, an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule; b) receiving, at one or more processors, a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles; c) executing, by the one or more processors, an objective function to measure a difference between the relative binding propensity and the observed allele frequency of the HLA allele; d) executing, by the one or more processors, an optimization model to minimize the objective function; e) determining an adjusted allele frequency of the HLA allele based on the optimization model and the observed allele frequency; and f) determining, by the one or more processors, that LOH has occurred when the adjusted allele frequency of the HLA allele is less than a predetermined threshold. In some embodiments, the HLA gene is a human HLA-A, HLA-B, or HLA-C gene. In some embodiments, the plurality of sequence reads was obtained by sequencing nucleic acids obtained from a sample comprising tumor cells and/or tumor nucleic acids. In some embodiments, the sample further comprises non-tumor cells.

In still some other aspects, provided herein are methods of identifying an individual having cancer where loss-of-heterozygosity (LOH) of a human leukocyte antigen (HLA) gene indicates a propensity of the individual with a particular type of disease to respond to a particular treatment. In some embodiments, the methods comprise: detecting LOH of the HLA gene in a sample from the individual, wherein LOH of the HLA gene is detected according to the method according to any of the embodiments described herein. In some embodiments, LOH of the HLA gene in the sample indicates that the individual is not likely to benefit from a treatment comprising an ICI. In some embodiments, detecting lack of LOH of the HLA gene in the sample indicates that the individual is likely to benefit from a treatment comprising an ICI. In some embodiments, the methods further comprise: detecting a tumor mutation burden (TMB) in a sample obtained from the individual. In some embodiments, the methods further comprise: acquiring knowledge of a high tumor mutation burden (TMB) in a sample obtained from the individual. In some embodiments, LOH of the HLA gene and high TMB indicate that the individual is likely to benefit from a treatment comprising an ICI. In some embodiments, LOH of the HLA gene and low TMB, or LOH of the HLA gene without high TMB, indicate that the individual is not likely to benefit from a treatment comprising an ICI.

In still some other aspects, provided herein are methods of selecting a therapy for an individual having cancer. In some embodiments, the methods comprise: detecting loss-of-heterozygosity (LOH) of a human leukocyte antigen (HLA) gene in a sample from the individual, wherein LOH of the HLA gene is detected according to the method according to any of the embodiments described herein. In some embodiments, LOH of the HLA gene in the sample indicates that the individual is not likely to benefit from a treatment comprising an ICI. In some embodiments, detecting lack of LOH of the HLA gene in the sample indicates that the individual is likely to benefit from a treatment comprising an ICI. In some embodiments, the methods further comprise: detecting a tumor mutation burden (TMB) in a sample obtained from the individual. In some embodiments, the methods further comprise: acquiring knowledge of a high tumor mutation burden (TMB) in a sample obtained from the individual. In some embodiments, LOH of the HLA gene and high TMB indicate that the individual is likely to benefit from a treatment comprising an ICI. In some embodiments, LOH of the HLA gene and low TMB, or LOH of the HLA gene without high TMB, indicate that the individual is not likely to benefit from a treatment comprising an ICI.

In still some other aspects, provided herein are methods of identifying one or more treatment options for an individual having cancer. In some embodiments, the methods comprise: (a) acquiring knowledge of loss-of-heterozygosity (LOH) of a human leukocyte antigen (HLA) gene in a sample from the individual, wherein LOH of the HLA gene is detected according to the method according to any of the embodiments described herein; and (b) generating a report comprising one or more treatment options identified for the individual based at least in part on said knowledge. In some embodiments, LOH of the HLA gene in the sample indicates that the individual is not likely to benefit from a treatment comprising an ICI. In some embodiments, the one or more treatment options do not include treatment comprising an ICI. In some embodiments, the methods comprise: (a) acquiring knowledge of lack of loss-of-heterozygosity (LOH) of a human leukocyte antigen (HLA) gene in a sample from the individual, wherein lack of LOH of the HLA gene is detected according to the method according to any of the embodiments described herein; and (b) generating a report comprising one or more treatment options identified for the individual based at least in part on said knowledge. In some embodiments, lack of LOH of the HLA gene in the sample indicates that the individual is likely to benefit from a treatment comprising an ICI. In some embodiments, the methods further comprise: detecting a tumor mutation burden (TMB) in a sample obtained from the individual. In some embodiments, LOH of the HLA gene and high TMB indicate that the individual is likely to benefit from a treatment comprising an ICI. In some embodiments, LOH of the HLA gene and low TMB, or LOH of the HLA gene without high TMB, indicate that the individual is not likely to benefit from a treatment comprising an ICI. In some embodiments, the methods further comprise acquiring knowledge of high TMB in a sample from the individual, and the one or more treatment options include treatment comprising an ICI.

In still some other aspects, provided herein are methods of selecting treatment for an individual having cancer. In some embodiments, the methods comprise acquiring knowledge of loss-of-heterozygosity (LOH) of a human leukocyte antigen (HLA) gene in a sample from an individual having cancer, wherein LOH of the HLA gene is detected according to the method according to any of the embodiments described herein. In some embodiments, responsive to the acquisition of said knowledge: (i) the individual is classified as a candidate not to receive treatment with an immune checkpoint inhibitor (ICI); (ii) the individual is identified as not likely to respond to a treatment that comprises an immune checkpoint inhibitor (ICI); and/or (iii) the individual is classified as a candidate to receive a treatment other than an immune checkpoint inhibitor (ICI). In some embodiments, the methods comprise acquiring knowledge of lack of loss-of-heterozygosity (LOH) of a human leukocyte antigen (HLA) gene in a sample from an individual having cancer, wherein lack of LOH of the HLA gene is detected according to the method according to any of the embodiments described herein. In some embodiments, responsive to the acquisition of said knowledge: (i) the individual is classified as a candidate to receive treatment with an immune checkpoint inhibitor (ICI); and/or (ii) the individual is identified as likely to respond to a treatment that comprises an immune checkpoint inhibitor (ICI). In some embodiments, the methods comprise: acquiring knowledge of LOH of a human leukocyte antigen (HLA) gene in a sample obtained from the individual and acquiring knowledge of a high tumor mutation burden (TMB) in a sample obtained from the individual. In some embodiments, responsive to the acquisition of said knowledge: (i) the individual is classified as a candidate to receive treatment with an immune checkpoint inhibitor (ICI); and/or (ii) the individual is identified as likely to respond to a treatment that comprises an immune checkpoint inhibitor (ICI).

In still some other aspects, provided herein are methods of predicting survival of an individual having cancer treated with an immune checkpoint inhibitor (ICI). In some embodiments, the methods comprise acquiring knowledge of loss-of-heterozygosity (LOH) of a human leukocyte antigen (HLA) gene in a sample from the individual, wherein LOH of the HLA gene is detected according to the method according to any of the embodiments described herein. In some embodiments, responsive to the acquisition of said knowledge, the individual is predicted to have shorter survival after treatment with the ICI, as compared to survival of an individual treated with the ICI whose cancer does not exhibit LOH of the HLA gene. In some embodiments, the methods comprise acquiring knowledge of lack of loss-of-heterozygosity (LOH) of a human leukocyte antigen (HLA) gene in a sample from the individual, wherein lack of LOH of the HLA gene is detected according to the method according to any of the embodiments described herein. In some embodiments, responsive to the acquisition of said knowledge, the individual is predicted to have longer survival after treatment with the ICI, as compared to survival of an individual treated with the ICI whose cancer exhibits LOH of the HLA gene. In some embodiments, the methods comprise acquiring knowledge of loss-of-heterozygosity (LOH) of a human leukocyte antigen (HLA) gene in a sample from the individual and acquiring knowledge of a high tumor mutation burden (TMB) in a sample obtained from the individual, wherein LOH of the HLA gene is detected according to the method according to any of the embodiments described herein. In some embodiments, responsive to the acquisition of said knowledge, the individual is predicted to have longer survival after treatment with the ICI, as compared to survival of an individual treated with the ICI whose cancer has LOH of the HLA gene without a high TMB.

In still some other aspects, provided herein are methods of monitoring an individual having cancer, comprising acquiring knowledge of loss-of-heterozygosity (LOH) of a human leukocyte antigen (HLA) gene in a sample from the individual, wherein LOH of the HLA gene is detected according to the method according to any of the embodiments described herein, and wherein responsive to the acquisition of said knowledge, the individual is predicted to have increased risk of recurrence, as compared to an individual whose cancer does not exhibit LOH of the HLA gene.

In still some other aspects, provided herein are methods of screening an individual having cancer, comprising acquiring knowledge of loss-of-heterozygosity (LOH) of a human leukocyte antigen (HLA) gene in a sample from the individual, wherein LOH of the HLA gene is detected according to the method according to any of the embodiments described herein, and wherein responsive to the acquisition of said knowledge, the individual is predicted to have increased risk of recurrence, as compared to an individual whose cancer does not exhibit LOH of the HLA gene.

In still some other aspects, provided herein are methods of evaluating an individual having cancer, comprising acquiring knowledge of loss-of-heterozygosity (LOH) of a human leukocyte antigen (HLA) gene in a sample from the individual, wherein LOH of the HLA gene is detected according to the method according to any of the embodiments described herein, and wherein the LOH of the HLA gene identifies the individual as having increased risk of recurrence, as compared to an individual whose cancer does not exhibit LOH of the HLA gene.

In some embodiments according to any of the embodiments described herein, LOH of the HLA gene is determined by: a) receiving, at one or more processors, an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule; b) receiving, at one or more processors, a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles; c) executing, by the one or more processors, an objective function that measures a difference between the relative binding propensity and the observed allele frequency of the HLA allele; d) executing, by the one or more processors, an optimization model configured to minimize the objective function; e) determining, by the one or more processors, an adjusted allele frequency of the HLA allele based on the optimization model and the observed allele frequency; and f) determining that LOH has occurred when the adjusted allele frequency of the HLA allele is less than a predetermined threshold.

In still some other aspects, provided herein are methods of treating or delaying progression of cancer. In some embodiments, the methods comprise: (1) detecting loss-of-heterozygosity (LOH) of a human leukocyte antigen (HLA) gene in a sample obtained from an individual, wherein LOH of the HLA gene is detected by: a) receiving, at one or more processors, an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule; b) receiving, at one or more processors, a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles; c) executing, by the one or more processors, an objective function to measure a difference between the relative binding propensity and the observed allele frequency of the HLA allele; d) executing, by the one or more processors, an optimization model to minimize the objective function; e) determining, by the one or more processors, an adjusted allele frequency of the HLA allele based on the optimization model and the observed allele frequency; and f) determining, by the one or more processors, that LOH has occurred when the adjusted allele frequency of the HLA allele is less than a predetermined threshold; and (2) based at least in part on detection of LOH of the HLA gene, administering an effective amount of a treatment other than an immune checkpoint inhibitor (ICI) to the individual. In some embodiments, the methods comprise: (1) detecting lack of loss-of-heterozygosity (LOH) of a human leukocyte antigen (HLA) gene in a sample obtained from an individual, wherein lack of LOH of the HLA gene is detected by: a) receiving, at one or more processors, an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule; b) receiving, at one or more processors, a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles; c) executing, by the one or more processors, an objective function to measure a difference between the relative binding propensity and the observed allele frequency of the HLA allele; d) executing, by the one or more processors, an optimization model to minimize the objective function; e) determining, by the one or more processors, an adjusted allele frequency of the HLA allele based on the optimization model and the observed allele frequency; and f) determining, by the one or more processors, that LOH has not occurred when the adjusted allele frequency of the HLA allele is greater than a predetermined threshold; and (2) based at least in part on detection of lack of LOH of the HLA gene, administering an effective amount of an immune checkpoint inhibitor (ICI) to the individual. In some embodiments, the methods comprise: (1) detecting loss-of-heterozygosity (LOH) of a human leukocyte antigen (HLA) gene in a sample obtained from an individual, wherein LOH of the HLA gene is detected by: a) receiving, at one or more processors, an observed allele frequency for an HLA allele, wherein the observed allele frequency corresponds to a frequency of nucleic acid(s) encoding at least a portion of the HLA allele as detected among a plurality of sequence reads corresponding to an HLA gene, wherein the plurality of sequence reads was obtained by sequencing nucleic acids encoding the HLA gene or a portion thereof as captured by hybridization with a bait molecule; b) receiving, at one or more processors, a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles; c) executing, by the one or more processors, an objective function to measure a difference between the relative binding propensity and the observed allele frequency of the HLA allele; d) executing, by the one or more processors, an optimization model to minimize the objective function; e) determining, by the one or more processors, an adjusted allele frequency of the HLA allele based on the optimization model and the observed allele frequency; f) determining, by the one or more processors, that LOH has occurred when the adjusted allele frequency of the HLA allele is less than a predetermined threshold: g) acquiring knowledge or, or detecting, high tumor mutational burden (TMB) in a sample obtained from the individual; and (3) based at least in part on detection of LOH of the HLA gene and high TMB, administering an effective amount of a treatment comprising an immune checkpoint inhibitor (ICI) to the individual.

In still some other aspects, provided herein is an immune checkpoint inhibitor (ICI) for use in method of treating or delaying progression of cancer in an individual, wherein loss-of-heterozygosity (LOH) of a human leukocyte antigen (HLA) gene has been detected in a sample obtained from the individual by: a) receiving, at one or more processors, an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule; b) receiving, at one or more processors, a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles; c) executing, by the one or more processors, an objective function to measure a difference between the relative binding propensity and the observed allele frequency of the HLA allele; d) executing, by the one or more processors, an optimization model to minimize the objective function; e) determining, by the one or more processors, an adjusted allele frequency of the HLA allele based on the optimization model and the observed allele frequency; and f) determining, by the one or more processors, that LOH has not occurred when the adjusted allele frequency of the HLA allele is greater than a predetermined threshold.

In still some other aspects, provided herein is an immune checkpoint inhibitor (ICI) for use in the manufacture of a medicament for treating or delaying progression of cancer in an individual, wherein loss-of-heterozygosity (LOH) of a human leukocyte antigen (HLA) gene has been detected in a sample obtained from the individual by: a) receiving, at one or more processors, an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule; b) receiving, at one or more processors, a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles; c) executing, by the one or more processors, an objective function to measure a difference between the relative binding propensity and the observed allele frequency of the HLA allele; d) executing, by the one or more processors, an optimization model to minimize the objective function; e) determining, by the one or more processors, an adjusted allele frequency of the HLA allele based on the optimization model and the observed allele frequency; and f) determining, by the one or more processors, that LOH has not occurred when the adjusted allele frequency of the HLA allele is greater than a predetermined threshold.

In still some other aspects, provided herein is an immune checkpoint inhibitor (ICI) for use in the manufacture of a medicament for treating or delaying progression of cancer in an individual, wherein loss-of-heterozygosity (LOH) of a human leukocyte antigen (HLA) gene and high tumor mutation burden (TMB) have been detected in a sample obtained from the individual by: a) receiving, at one or more processors, an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule; b) receiving, at one or more processors, a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles; c) executing, by the one or more processors, an objective function to measure a difference between the relative binding propensity and the observed allele frequency of the HLA allele; d) executing, by the one or more processors, an optimization model to minimize the objective function; e) determining, by the one or more processors, an adjusted allele frequency of the HLA allele based on the optimization model and the observed allele frequency; f) determining, by the one or more processors, that LOH has occurred when the adjusted allele frequency of the HLA allele is greater than a predetermined threshold; and g) detecting a high tumor mutation burden (TMB) in a sample obtained from the individual.

In still some other aspects, provided herein is a non-transitory computer readable storage medium comprising one or more programs executable by one or more computer processors for performing a method, comprising: receiving, using the one or more processors, an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule; receiving, using the one or more processors, a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles; executing, using the one or more processors, an objective function to measure a difference between the relative binding propensity and the observed allele frequency of the HLA allele; executing, using the one or more processors, an optimization model to minimize the objective function; determining, using the one or more processors, an adjusted allele frequency of the HLA allele based on the optimization model and the observed allele frequency; and determining, using the one or more processors, that LOH has occurred when the adjusted allele frequency of the HLA allele is less than a predetermined threshold. In still some other aspects, provided herein is a system comprising one or more processors; and a memory configured to store one or more computer program instructions, wherein the one or more computer program instructions when executed by the one or more processors are configured to: determine an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule; determine a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles; execute an objective function to measure a difference between the relative binding propensity and the observed allele frequency of the HLA allele; execute an optimization model to minimize the objective function; determine an adjusted allele frequency of the HLA allele based on the optimization model and the observed allele frequency; and determine that LOH has occurred when the adjusted allele frequency of the HLA allele is less than a predetermined threshold. In some embodiments, the HLA gene is a human HLA-A, HLA-B, or HLA-C gene. In some embodiments, the plurality of sequence reads was obtained by sequencing nucleic acids obtained from a sample comprising tumor cells and/or tumor nucleic acids. In some embodiments, the sample further comprises non-tumor cells. In some embodiments, the sample is from a tumor biopsy or tumor specimen. In some embodiments, the sample comprises tumor cell-free DNA (cfDNA). In some embodiments, the sample comprises fluid, cells, or tissue. In some embodiments, the sample comprises blood or plasma. In some embodiments, the sample comprises a tumor biopsy or a circulating tumor cell. In some embodiments, the sample is a nucleic acid sample. In some embodiments, the nucleic acid sample comprises mRNA, genomic DNA, circulating tumor DNA, cell-free DNA, or cell-free RNA. In some embodiments, the method further comprises using the one or more processors, acquiring knowledge of or detecting tumor mutational burden (TMB) from a plurality of sequence reads, wherein the plurality of sequence reads was obtained by sequencing nucleic acids at least a portion of a genome. In some embodiments, the TMB is determined based on a number of non-driver somatic coding mutations per megabase of genome sequenced.

In still some other aspects, provided herein are methods for detecting loss-of-heterozygosity (LOH) of a human leukocyte antigen (HLA) gene, comprising: (1) providing a plurality of nucleic acids obtained from a sample from an individual, wherein the plurality of nucleic acids comprises nucleic acids encoding an HLA gene; (2) optionally, ligating one or more adaptors onto one or more nucleic acids from the plurality; (3) amplifying nucleic acids from the plurality; (4) capturing a plurality of nucleic acids corresponding to the HLA gene, wherein the plurality of nucleic acids corresponding to the HLA gene is captured from the amplified nucleic acids by hybridization with a bait molecule: (5) sequencing, by a sequencer, the captured nucleic acids to obtain a plurality of sequence reads corresponding to the HLA gene; fitting, by one or more processors, one or more values associated with one or more of the plurality of sequence reads to a model; and (6) based on the model, detecting LOH of the HLA gene and a relative binding propensity for an HLA allele of the HLA gene. In some embodiments, LOH of the HLA gene and relative binding propensity for an HLA allele of the HLA gene are detected by a) obtaining an observed allele frequency for an HLA allele, wherein observed allele frequency corresponds to frequency of nucleic acid(s) encoding at least a portion of the HLA allele as detected among the plurality of sequence reads corresponding to the HLA gene; b) obtaining a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles; c) applying an objective function to measure a difference between the relative binding propensity and the observed allele frequency of the HLA allele; d) applying an optimization model to minimize the objective function; e) determining an adjusted allele frequency of the HLA allele based on the optimization model and the observed allele frequency; and f) determining that LOH has occurred when the adjusted allele frequency of the HLA allele is less than a predetermined threshold. In some embodiments, the methods further comprise, based at least in part on detection of LOH of the HLA gene, administering an effective amount of a treatment other than an immune checkpoint inhibitor (ICI) to the individual. In some embodiments, the methods further comprise, based at least in part on detection of LOH of the HLA gene, recommending a treatment other than an immune checkpoint inhibitor (ICI). In some embodiments, the methods further comprise detecting, or acquiring knowledge of, a high tumor mutational burden (TMB) in the sample (or a second sample obtained from the individual). In some embodiments, the methods further comprise, based at least in part on detection of LOH of the HLA gene and high TMB, administering an effective amount of an immune checkpoint inhibitor (ICI) to the individual. In some embodiments, the methods further comprise, based at least in part on detection of LOH of the HLA gene and high TMB, recommending a treatment comprising an immune checkpoint inhibitor (ICI) to the individual. In some embodiments, the HLA gene is a human HLA-A, HLA-B, or HLA-C gene. In some embodiments, the methods further comprise, prior to (1), extracting the plurality of nucleic acids from the sample. In some embodiments, the sample comprises tumor cells and/or tumor nucleic acids. In some embodiments, the sample further comprises non-tumor cells. In some embodiments, the sample is from a tumor biopsy or tumor specimen. In some embodiments, the sample comprises tumor cell-free DNA (cfDNA). In some embodiments, the sample comprises fluid, cells, or tissue. In some embodiments, the sample comprises blood or plasma. In some embodiments, the sample comprises a tumor biopsy or a circulating tumor cell. In some embodiments, the sample is a nucleic acid sample. In some embodiments, the nucleic acid sample comprises mRNA, genomic DNA, circulating tumor DNA, cell-free DNA, or cell-free RNA. In some embodiments, the TMB is determined based on a number of non-driver somatic coding mutations per megabase of genome sequenced.

In some embodiments according to any of the embodiments described herein, the ICI comprises a PD-1 inhibitor, a PD-L1 inhibitor, or a CTLA-4 inhibitor. In some embodiments, the methods further comprise detecting a tumor mutation burden (TMB) in a sample obtained from the individual. In some embodiments, LOH of the HLA gene in the sample and high TMB identify the individual as one who may benefit from the treatment comprising an ICI. In some embodiments, an effective amount of the immune checkpoint inhibitor (ICI) is administered to the individual based at least in part on LOH of the HLA gene in the sample and high TMB. In some embodiments, high TMB refers to a TMB of greater than or equal to 10 mutations/Mb or greater than or equal to 13 mutations/Mb. In some embodiments, the HLA gene is an HLA-I gene.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE FIGURES

FIG. 1 is a schematic depiction of a hybrid capture process.

FIG. 2 illustrates the result of a bias removal process.

FIG. 3A depicts an exemplary device, in accordance with some embodiments.

FIG. 3B depicts an exemplary system, in accordance with some embodiments.

FIGS. 4A-D depict properties and methods of HLA-I detection. FIG. 4A is a schematic representation of somatic HLA-I LOH, PD-L1 expression, and tumor mutational burden (TMB) in the context of other oncogenic processes (adapted from Hegde, P. S. and Chen, D. S. (2020) Immunity 52:17-35). FIG. 4B illustrates the relation of HLA-I LOH to immune response. HLA-I LOH is related to TMB through neoantigens and to PD-L1 as an evasion mechanism (see McGranahan, N. et al. (2017) Cell 171:1259-1271). FIG. 4C is a schematic overview of the computational pipeline used for detection of somatic loss of heterozygosity as well as germline homozygosity of the HLA-I locus from mixed tumor-normal next-generation sequencing results. FIG. 4D illustrates methodological considerations for detection of HLA-I LOH due to baiting effects, including the bait/target sequence divergence effects in BAF (upper), and the modeled BAF accounting for sequencing (lower).

FIGS. 4E-4G show the effects of hybridization on HLA baiting efficiency. FIG. 4E provides an example of how hybrid-capture can pull down HLA target sequences with different efficiency. Shown are allele frequency (AF) of the HLA-A*31:01 allele before (top; median AF=0.3) and after (bottom; median AF=0.5) adjusting for hybrid-capture binding affinity bias in samples with HLA-A*31:01 and HLA-A*11:01 alleles. FIG. 4F shows a dendrogram of representative sequences for each known two-digit haplotype of HLA-A. A matrix of all pairwise sequence distances was used to cluster the haplotypes. The k affinity constants for haplotypes on the left were all greater than or equal to 1, while the k constants for sequences on the right were all less than 1. FIG. 4G shows a dendrogram of representative sequences for each known two-digit haplotype of HLA-A. A matrix of all pairwise sequence distances was used to cluster the haplotypes. The k affinity constants for haplotypes on the left were all greater than or equal to 1, while the k constants for sequences on the right were greater than 0.7 or between 0.7 and 0.9. The dot on the left axis represents the sequence of a specific bait molecule used for capture of various HLA alleles.

FIGS. 5A & 5B depict survival probability for known genomic associations in clinic-genomic databases (CGDBs). FIG. 5A shows the survival curves for high (≥10 mutations/Mb) and low (<10 mutations/Mb) tumor mutational burden (TMB). TMB high was positively associated with survival (HR=0.76, P=0.007). FIG. 5B shows the survival curves for loss of SK/I or KEAP1. Loss of STK11 or KEAP1 was negatively associated with survival (HR=1.3, P=0.009) in a prior analysis of a second-line checkpoint inhibitor monotherapy treated non-squamous non-small cell lung cancer (NSCLC) cohort (N=652).

FIGS. 6A-6D show that somatic HLA-I LOH and TMB are independent and significant predictors patient survival in immune checkpoint inhibitor (ICI)-treated NSCLC. FIG. 6A shows the enrichment of sample attributes, including genomic driver alterations, in non-squamous NSCLC samples with HLA-I LOH (right, n=2,769) and without evidence of HLA-I LOH (left, n=10,471). TMB high: ≥10 muts/Mb; PD-L1 positive; ≥1% tumor proportion score. Statistics conducted by Fisher's Exact and only highly significant (P<0.01) associations are labeled. FIG. 6B shows the overall survival of non-squamous NSCLC patients from start of second-line ICI monotherapy, stratified by HLA-I LOH status. Median overall survival (mOS) for HLA-I intact (n=180) was 11.3 months [8.2-15.3] and HLA-I LOH (n=60) was 8.0 months [5.2-13.1]. HR for HLA-I intact=0.68 [0.49-0.95], P=0.02. FIG. 6C shows the lack of effect of biopsy timing in CGDB. FIG. 6D depicts overall survival of non-squamous NSCLC patients from start of second-line ICI monotherapy, stratified by HLA-I LOH and TMB status (TMB high: ≥10 muts/Mb, TMB low: <10 muts/Mb). TMB high, HLA-I intact (n=82) mOS 14.09 months [9.0-21.1]. TMB high, HLA-I LOH (n=31) mOS 10.87 months [6.60-20.0]. TMB low, HLA-I intact (n=98) mOS 9.59 months [6.18-14.8]. TMB low, HLA-I LOH (n=29) mOS 4.83 months [2.86-12.6]. HR for TMB high=0.74 [0.54-0.99], P=0.046. HR for HLA-I intact=0.65 [0.47-0.91], P=0.013. Somatic HLA-I LOH and TMB as independent and significant predictors of patient survival in ICI-treated NSCLC.

FIGS. 7A & 7B show the impact of HLA-I germline zygosity on patient survival in ICI-treated NSCLC. FIG. 7A depicts the overall survival of all non-squamous NSCLC patients from start of second-line ICI monotherapy, stratified by number of germline unique HLA-I alleles. The mOS was the same for both cohorts, regardless of germline HLA-I allele count (germline HLA-T allele count=6 (n=182): mOS 10.8 [7.49-14.0]; germline HLA-I allele count <6: mOS 10.8 [4.80-18.3]; P=0.6). FIG. 7B depicts the overall survival of non-squamous NSCLC patients with no evidence of somatic HLA-I LOH from start of second-line ICI monotherapy, stratified by number of germline unique HLA-I alleles. The mOS for patients with a germline HLA-I allele count of 6 (n=141) was 11.9 months [8.84-15.90] and the mOS for patients with a germline allele count less than 6 (n=39) was 7.1 months [3.68-19.20], P=0.9. HLA-I germline zygosity on patient survival in ICI-treated NSCLC.

FIGS. 7C & 7D show overall survival of non-squamous NSCLC patients in the real-world clinico-genomic cohort from start of second-line ICI monotherapy. FIG. 7C shows overall survival stratified by the most statistically significant TMB (muts/Mb) and HLA-I status combination. mOS for patients that were any TMB, HLA-I Intact or TMB≥13, HLA-I LOH (n=203) was 12.2 months [9.1-15.3]. The mOS for patients that were TMB<13, HLA-I LOH (n=37) was 6.0 months [2.9-8.9]. HR for any TMB, HLA-I Intact; TMB≥13, HLA-I LOH=0.45 [0.31-0.66], P=0.00004. FIG. 7D shows overall survival stratified by HLA-I LOH and TMB status across multiple TMB thresholds (1-20 mut/Mb). For each threshold, TMB high ≥TMB threshold and TMB low <TMB threshold. The hazard ratio is derived from multivariate Cox proportional hazards models controlled for TMB at each TMB threshold.

FIGS. 8A-8F illustrate the pan-cancer landscape of somatic HLA-I LOH. FIG. 8A depicts the prevalence of HLA-I LOH across 59 different solid tumor types in 83,664 unique patient samples. The number of patients within each tumor type are summarized in Table 2. FIG. 8B shows the prevalence of HLA-I LOH in microsatellite stable (MSS) as compared to microsatellite instable (MSI-H) samples in tumor types with high frequency (≥3%) of microsatellite instability. Number of samples (MSS, MSI): small intestine (n=420, n=21), gastric (n=1100, n=49), colorectal (n=9787, n=332), endometrial (n=1883, n=330), uterine (n=385, n=20). Statistics conducted by Fisher's Exact. FIG. 8C shows the prevalence of HLA-I LOH within breast cancer molecular subtypes. All breast (n=9686), HER+(n=281), ER+/HER2− (n=731), triple negative (n=631). Statistics conducted by Chi-square. FIG. 8D shows the prevalence of HLA-I LOH in PD-L1 positive (≥1% tumor proportion score, n=3271) as compared to PD-L1 negative (<1% tumor proportion score, n=9920) samples (top). Statistics conducted by Fisher's Exact. FIG. 8D also shows the association between the prevalence of HLA-I LOH and the prevalence of PD-L1 positivity within each tumor type (bottom). Association is fitted with a linear regression. FIG. 8E shows the prevalence of HLA-I LOH in TMB high (≥10 muts/Mb, n=13393) as compared to TMB low (<10 muts/Mb, n=70263) samples (top). Statistics conducted by Fisher's Exact. FIG. 8E also shows the association between the prevalence of HLA-I LOH and the prevalence of TMB high samples within each tumor type (bottom). Association is fitted with a regression model (e.g., loess regression, quadratic regression, etc.). For tumor types with high rates of microsatellite instability (small intestine, gastric, colorectal, endometrial, and uterine), MSS and MSI-H samples are represented separately in the bottom graphs of FIGS. 8D & 8E. Significant (P<0.05) associations are labeled with an asterisk. FIG. 8F shows the association of HLA-I LOH with TMB and PD-L1.

FIGS. 9A & 9B show the association of DAX loss of function mutations and HLA-I LOH in tumor types with low rates of PD-L1 positivity and low TMB. FIG. 9A shows the enrichment of sample attributes, including genomic driver alterations, in pancreatic islet cell samples with HLA-I LOH (right, n=97) and without evidence of HLA-I LOH (left, n=157). FIG. 9B shows the enrichment of sample attributes, including genomic driver alterations, in adrenocortical carcinoma samples with HLA-I LOH (right, n=62) and without evidence of HLA-I LOH (left, n=110). Statistics conducted by Fisher's Exact and only significant (P<0.05) associations are labeled.

FIGS. 10A & 10B show results linking somatic HLA-I LOH to immune evasion in samples with tumor antigen presentation. FIG. 10A shows a neoantigen prediction of recurrent driver mutations conducted by NetMHCpan. Predicted neoantigens are listed as gene:protein effect and the percent of times the predicted presenting allele was either lost or kept during a loss of heterozygosity event is shown. Only neoantigens involved with >5 events are included. Statistics conducted by Binomial Test and significance determined as P<0.05. FIG. 10B depicts the prevalence of HLA-I LOH in tumor types with known oncoviral associations. HPV: human papillomavirus; EBV: Epstein-Barr virus; HBV: hepatitis B virus. Number of samples (virus-positive, virus-negative): head and neck squamous cell carcinoma (SqCC) (n=363, n=771), cervical (n=141, n=121), gastric (n=189, n=1018), nasopharyngeal (n=50, n=38), hepatocellular (n=64, n=506). Statistics conducted by Fisher's Exact and significant (P<0.05) associations are labeled with an asterisk. Somatic HLA-I LOH is a potential mechanism for immune evasion in samples with tumor antigen presentation

FIGS. 11A & 11B show the enrichment of genomic alterations in samples with somatic HLA-I LOH. FIG. 11A illustrates the enrichment of genomic alterations in tumor types with HLA-I LOH (“Enriched in HLA-I LOH Samples”) and without evidence of HLA-I LOH (“Enriched in HLA-I Intact Samples”). Tumor types in the top quartile overall in prevalence of TMB high samples (≥10 muts/Mb), PD-L1 positivity (≥1% tumor proportion score), APOBEC mutational signature, tobacco mutational signature, and UV mutational signature are shown. Only genes with enrichment in at least six different tumor types are included. FIG. 11B depicts tumor type enrichment in samples with select genomic mutations, stratified by HLA-I LOH status. The statistics shown in FIGS. 11A & 11B were conducted by Fisher's Exact, and only significant (P<0.05) associations are shown.

FIG. 12 depicts a block diagram of an exemplary process for detecting loss-of-heterozygosity (LOH) of a human leukocyte antigen (HLA) gene, in accordance with some embodiments.

FIG. 13 depicts a block diagram of an exemplary process for identifying relative binding propensities of different alleles of a polymorphic gene to a bait molecule, in accordance with some embodiments.

FIG. 14 depicts a block diagram of an exemplary process for determining allele frequency, in accordance with some embodiments.

### DETAILED DESCRIPTION

It is often an important clinical goal to assess whether a subject (e.g., a human or other animal) has experienced a loss of heterozygosity (“LOH”) in one or more genes. One way to determine LOH of a particular gene in a subject is to use a hybrid capture process. As used herein, LOH can refer to copy-loss LOH and/or copy-neutral LOH.

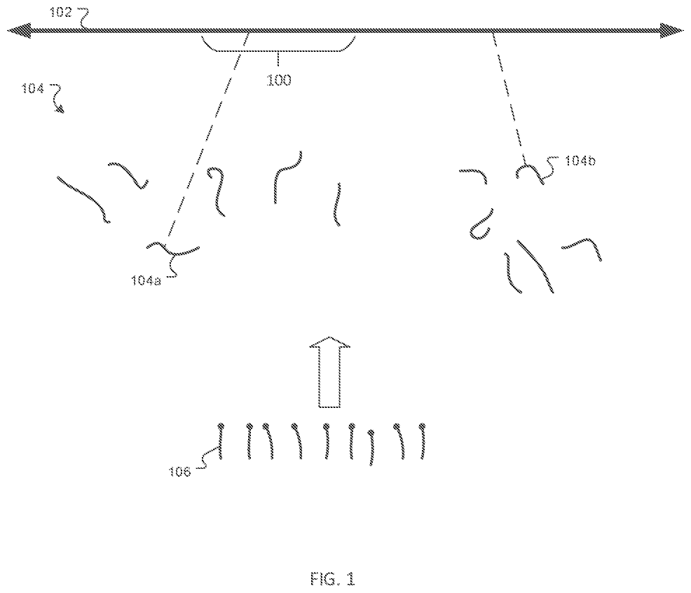

FIG. 1 illustrates a prior art hybrid capture process. Further details about this and other hybrid capture processes can be found in U.S. Pat. No. 9,340,830, the entirety of which is incorporated by reference herein.

A population of DNA fragments 104 from the subject is prepared, some of which correspond to the gene of interest 100 within the subject's genome 102. If the subject is heterozygous at the gene of interest 100 then population of DNA fragments 104 will comprise different alleles (one from each parent), in roughly equal amounts. On the other hand, if the subject has undergone LOH, then one of the parent's alleles will be absent or significantly decreased in the population of on-target fragments 104a.

Thus, consistent with the hybrid capture approach, a population of bait molecules 106 corresponding to the gene of interest 100 are introduced to the population of the subject's DNA fragments 104. The bait molecules 106 will bond with “on-target” fragments 104a—that is, DNA fragments 104 that originate from the gene of interest 100. Conversely, the bait molecules 106 will not bond with “off-target” fragments 104b.

After sufficient time to allow such bonding to happen, the fragment/bait hybrids are captured and the remaining fragments are discarded. The captured hybrids are then sequenced to determine which alleles are present, and their relative frequencies. If the allele frequencies are sufficiently close to equal, then the patient can be determined to be heterozygous. If one allele frequency is sufficiently low, then the patient can be determined to have undergone LOH in the gene of interest 100.

This relatively straightforward process can be complicated by a number of factors. First, the patient sample may be of a mixed nature. For example, if the sample comes from a tumor biopsy, the sample may contain both normal, healthy cells from the patient as well as cancerous cells from a tumor. Second, some cancer cells may exhibit aneuploidy, in which the cancer cells have a greater or lesser than typical number of duplicate chromosomes. If one or both of these factors are present, they may change the expected allele frequencies for either a heterozygous subject or a subject that has experienced LOH.

Techniques have been developed to assess LOH even in the presence of these factors. One approach is to introduce extra parameters, including tumor purity (i.e., the proportion of the sample that contains tumor cells vs. healthy cells) and tumor ploidy (i.e., the number of duplicate chromosomes the tumor cells possess) into a mathematical calculation similar to that described above. An example of this approach is described, for example, in McGranahan et al., Allele-Specific HLA Loss and Immune Escape in Lung Cancer Evolution (Cell, 2017 Nov. 30; 171(6): 1259-1271.e11), the entirety of which is incorporated by reference herein.

However, the techniques disclosed therein still are prone to a statistical bias that may skew the assessment of measured allele frequencies, especially for genes of interest 100 exhibiting a large degree of polymorphism.

One such family of genes are the Human Leukocyte Antigen (“HLA”) genes. These genes are responsible, in part, for regulating the immune system in humans. These functions are spread across several sites within the human genome 102, but even at certain particular sites there can be up to thousands of possible alleles.

Consequently, in the hybrid capture process described above, a particular bait molecule 106 will not enjoy perfect complementarity with most possible alleles. Moreover, different alleles may compete with each other to bind with the same bait molecule. In turn, these phenomena can effect (sometimes profoundly) the propensity of an on-target fragment 104a successfully binding to a bait molecule 106. This results in that particular allele being under-sampled or over-sampled by the capture process, and therefore in a measured allele frequency that is artificially high or low, and in some cases incorrectly making a determination whether the subject has experienced LOH or not.

One approach to mitigate this sampling error is to empirically determine relative binding propensities of the various alleles to a particular bait molecule; e.g., if a sample of subject DNA fragments 104 truly included equal proportions of on-target fragments 104a from two different alleles, then it may be empirically determined what actual allele frequencies result from a hybrid capture process using a particular bait molecule 106. Because the binding propensities are due, at least in part, to inter-allelic competition, this determination may be made on an allele-pair-by-allele-pair basis, not just on an allele-by-allele basis. If those relative binding propensities were known, then the sampling bias of subsequent hybrid capture processes with those alleles and bait molecules can be corrected by scaling the observed allele frequencies. For example, an objective function can be applied to measure a difference between the relative binding propensity and the observed allele frequency of a given allele.

But for highly polymorphic genes like HLA, this approach may not be practical, insofar as there are too many allele pairs to determine all the relative binding propensities. However, the following techniques can be useful if only a subset of relative binding propensities are known.

In what follows, suppose the gene of interest 100 is a polymorphic gene having n possible alleles. The relative binding propensity of alleles i and j to a bait molecule is given by:

$$\frac{{k}_{i}}{{k}_{j}} = \frac{{AF}_{i}}{{AF}_{j}}$$

Where kᵢ and kⱼ are the relative binding propensities of alleles i and j, respectively, and AFᵢ and AFⱼ represent the corresponding allele frequencies of alleles i and j respectively.

Note that since AFᵢ and AFⱼ are determined in part by the interaction of their corresponding alleles, these numbers should be understood to describe allele frequencies of alleles i and j only in the presence of the other allele. In other words, if i, j, and k are distinct, then AFᵢ might be different in the presence of allele j vs. allele k. In some implementations, it is convenient to linearize these equations by taking the logarithm of the expression above, obtaining:

log(kᵢ)−log(kⱼ)=log(AFᵢ)−log(AFⱼ)

With n alleles, there are a total of n(n−1) pairs of alleles. Thus, one may obtain a system of n(n−1) linear equations of the form above, that express relative binding propensities of alleles in terms of observed allele frequencies. If empirical allele frequency data is available for all possible pairs of alleles, this system may be solved in a straightforward manner.

However, even if empirical allele frequency data is available for only a subset of the possible pairs, a useful estimate may still be made by an error-minimization approach. The above system of equations can be expressed as the form Ax=b, where A is a matrix with n(n−1) rows and n columns, in which all entries are 0 in each row, except for a 1 term in one column and a −1 term in another, such that no two rows are equal. The vector x is a column vector having the component log kᵢ in the i-th position, and b is a column vector with each component of the form log(AFₙ)−log(AFₘ), with the values of n and m corresponding to the positions of the nonzero terms of A in the corresponding row. In some implementations, a row of the matrix can be modified so its only nonzero term is equal to 1, in some position (column m, for example). This is tantamount to arbitrarily setting the relative binding propensity of the bait molecule to allele m equal to 1, thereby setting the scale against which other relative binding propensities are measured.

If not all kᵢ and/or AFᵢ terms are known, then a practical estimate may be arrived at by defining error terms E_{i,j} by the expression

$${E}_{ij} = \frac{{k}_{i}}{{k}_{j}} - \frac{{AF}_{i}}{{AF}_{j}}$$

and selecting the unknown kᵢ and/or AFᵢ terms to minimize the total error (or some mathematical function thereof; e.g., an absolute value, the squared value, etc.). In some implementations, this minimization may be performed subject to other constraints, e.g. the requirement that the median value of all the kᵢ terms is equal to 1. In some implementations, the error is minimized by performing a least-squares optimization, although other optimization methods are suitable.

With the kᵢ terms having been either empirically determined or computed according to the previous paragraph, they can be used to re-scale the raw, measured allele frequencies from a hybrid capture process, thereby mitigating sampling bias that existed as a result of the factors described above.

Certain aspects of the present disclosure relate to methods for determining allele frequency. In some embodiments, the methods comprise: a) obtaining an observed allele frequency for an allele of a gene, wherein the observed allele frequency corresponds to frequency of nucleic acid(s) encoding at least a portion of the allele as detected among a plurality of sequence reads corresponding to the gene, wherein the plurality of sequence reads was obtained by sequencing nucleic acids encoding the gene or a portion thereof as captured by hybridization with a bait molecule; b) obtaining a relative binding propensity for the allele to the bait molecule, wherein the relative binding propensity of the allele corresponds to propensity of nucleic acid encoding at least a portion of the allele to bind the bait molecule in the presence of nucleic acids encoding portions of one or more other alleles of the gene; c) applying an objective function to measure a difference between the relative binding propensity and the observed allele frequency of the allele; d) applying an optimization model to minimize the objective function; and e) determining an adjusted allele frequency of the allele based on the optimization model and the observed allele frequency.

Optimization Modeling

Optimization refers to the method and process of working toward a solution which may be the best available solution, a preferred solution, or a solution that offers a specific benefit within a range of constraints; or continually improving; or refining; or searching for a high point or maximum (or a low point or a minimum) for an objective; or processing to reduce a penalty function or cost function; etc. In optimization modeling, the objective is often to minimize the model error, also known as the residuals of the model (a residual being the difference between an observed value and the fitted value provided by the model).

Generally, an optimization model has three main components: a) an objective function, which is the function that needs to be optimized (e.g., minimize error of parameter estimation of the model); b) a collection of variables, wherein the solution to the optimization problem is the set of values of the variables for which the objective function reaches its optimal value; and c) a collection of constraints that restrict the values of the variables. Various optimization models are known in the art and may be used in the methods of the present disclosure. One of skill in the art would be able to ascertain the suitable optimization model to use according to their specific needs and criteria. Examples of optimization models in the art include, but are not limited to, least squares regression models, logistic regression models, quadratic regression models, loess regression models, Bayesian ridge regression models, lasso regression models, elastic net regression models, decision tree models, gradient boosted tree models, neural network models, and support vector machine models. Further descriptions regarding optimization modeling can be found, e.g., in Yang, X. (2008). Introduction to mathematical optimization. From Linear Programming to Metaheuristics; Allaire, G., & Allaire, G. (2007). Numerical analysis and optimization: an introduction to mathematical modelling and numerical simulation. Oxford university press; Pedregal, P. (2006). Introduction to optimization (Vol. 46). Springer Science & Business Media; Chong, E. K., & Zak, S. H. (2004). An introduction to optimization. John Wiley & Sons; and the like.

Allele Frequencies

In some embodiments, the optimization model comprises allele frequencies as model variables. Allele frequency is the frequency of an allele (i.e., a variant of nucleotide sequence) at a genomic locus in a population of alleles, expressed as a fraction or percentage. When the population of alleles refers to a population of alleles of one individual subject, the frequency of an allele can be calculated as the ratio of the sequence counts of the allele to the total sequence counts of all alleles at a given genomic locus of the individual subject. In this sense, the allele frequency represents the allelic composition of the individual at the genomic locus, from which the zygosity (e.g. homozygous or heterozygous) can be inferred. For instance, for a diploid individual subject, such as a human:
1) if the allele frequency of an allele has a value of, or reasonably close to (e.g., within the statistical confidence interval of), 0, then the individual subject is considered homozygous null (also known as nullizygous) for this allele;
2) if the allele frequency of an allele has a value of, or reasonably close to (e.g., within the statistical confidence interval of), 0.5, then the individual subject is considered heterozygous for this allele; and
3) if the allele frequency of an allele has a value of, or reasonably close to (e.g., within the statistical confidence interval of), 1, then the individual subject is considered homozygous for this allele.

In some embodiments, the allele frequency is an observed allele frequency, corresponding to relative frequency of nucleic acid(s) encoding at least a portion of the allele as detected among the plurality of sequence reads, as compared to a reference value. In some embodiments, the reference value is a total number of sequence reads. In some embodiments, the reference value is a number of sequence reads corresponding to a reference gene, or a function thereof, such as reads per million mapped reads (RPM) or counts per million mapped reads (CPM).

In some embodiments, the allele frequency can be expressed as the relative binding propensity. In a hybrid capture-based sequencing process, the relative binding propensity corresponds to the likelihood of one allele binding to the bait molecule in the presence of one or more other alleles. Accordingly, in some embodiments, an optimization model is applied to an objective function that measures a difference between the relative binding propensity of one allele and the observed allele frequency of the allele.

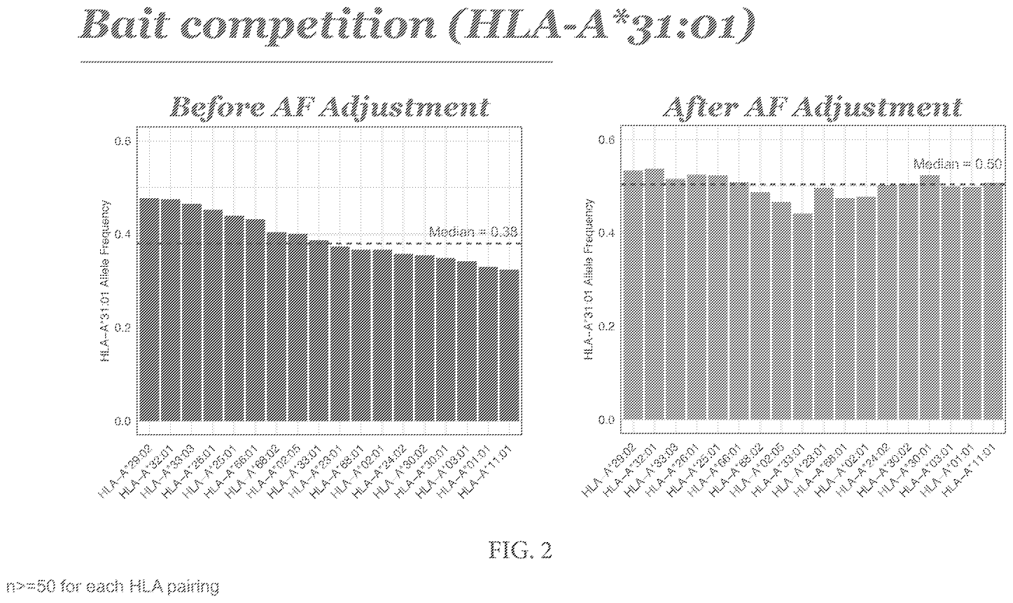

By way of example, FIG. 2 illustrates the result of such a scaling for various HLA-A alleles. The bar chart on the left indicates raw allele frequencies from heterozygous subjects of the HLA-A*31:01 allele in the presence of various other HLA-A alleles indicated on the horizontal axis. The median allele frequency is 0.38, indicating that HLA-A*31:01 is typically under-sampled in the presence of the indicated other alleles. After correcting the bias, the chart on the right indicates a median allele frequency of 0.5, which is more consistent with a heterozygous sample population.

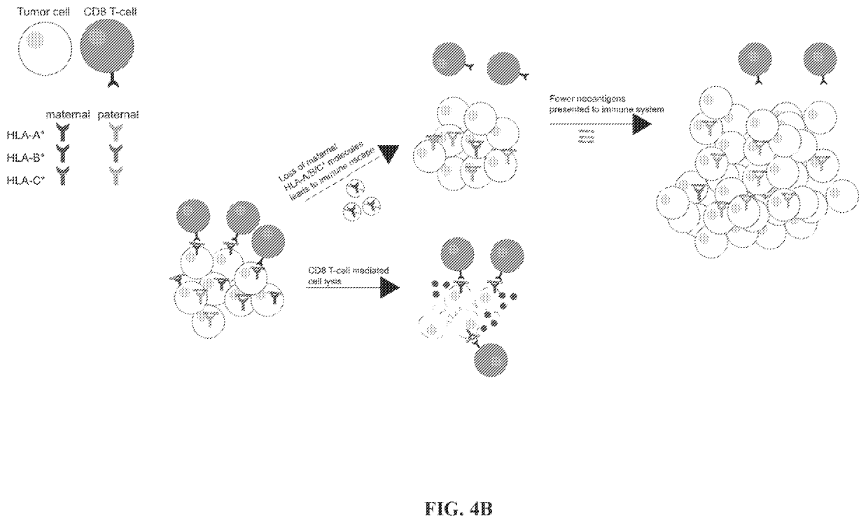

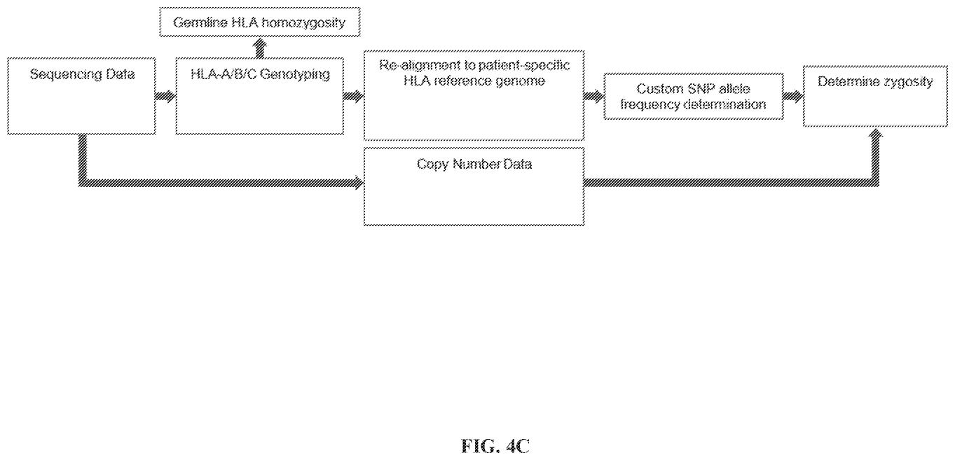

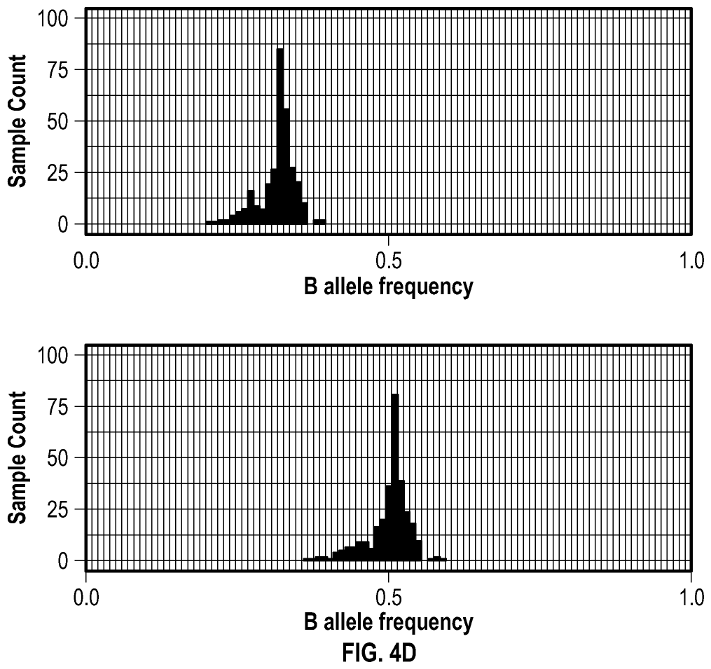

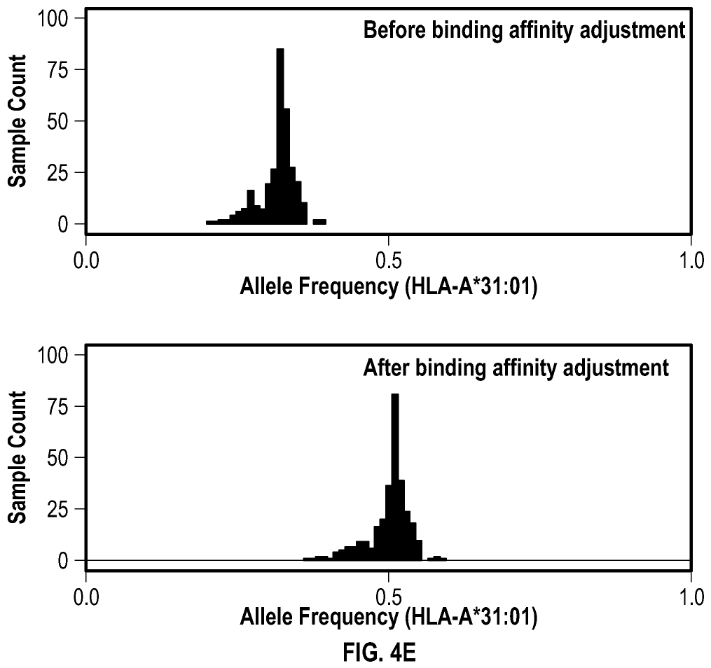

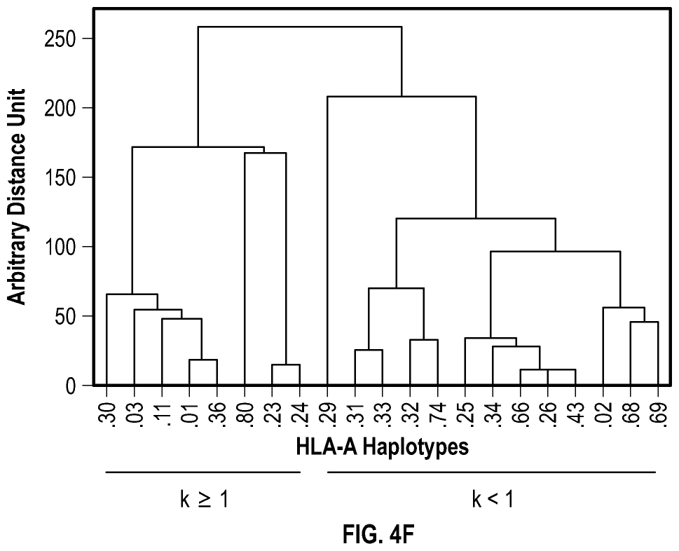

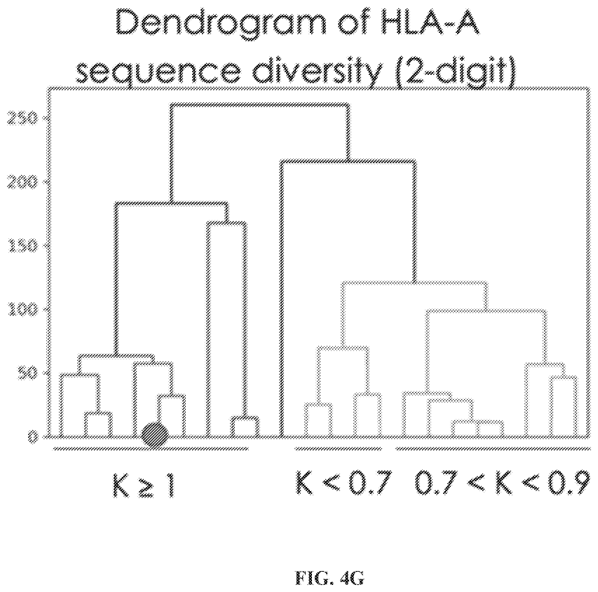

FIG. 4D illustrates the effect of adjusted allele frequencies for use in determining loss-of-heterozygosity (LOH) for the human leukocyte antigen class I (HLA-I) gene in a population of individuals. The X-axis shows the B allele frequency (BAF) for each individual in the population, wherein the B allele refers to the non-reference allele, or the minor allele. The Y-axis shows the sample count of the BAF in the population. FIG. 4D shows that after adjusting the allele frequencies using the methods of the present disclosure, the median allele frequency is adjusted from around 0.32 (upper panel) to around 0.5 (lower panel), suggesting most of the population of individuals are heterozygous for the HLA-I gene.

As shown in FIG. 4G, particular alleles (or fragments thereof) may have a range of relative binding propensities to a particular bait molecule. In order to improve capture of sequences representing the full polymorphic variation of the gene, one may wish to select one or more additional bait molecule(s), particularly those that have improved binding propensities to alleles with a lower relative binding propensity to the original bait molecule.

As such, in some embodiments, the methods of the present disclosure may include obtaining an observed allele frequency for two or more alleles of a gene; obtaining a relative binding propensity for two or more alleles of a gene to a specific bait molecule; and/or identifying or selecting the sequence of a second bait molecule. In some embodiments, one or more alleles of the gene with a lower relative binding propensity to a first bait molecule may have a higher binding propensity to the second bait molecule than to the first bait molecule. For example, the second bait molecule can comprise a sequence complementary to at least a portion of one of the lower-binding alleles of the gene, or to a sequence (e.g., a consensus sequence) based on complementarity or binding to the sequence(s) of one or more lower-binding alleles of the gene. This allows for bait selection based on the sequences of lower-binding alleles of a polymorphic gene, e.g., in order to sample the diversity of the gene more comprehensively or with less bias (e.g., based on hybrid capture).

Least Squares Optimization

In some embodiments, the optimization model is a least squares optimization model. A least squares optimization model is a regression optimization model wherein the objective function is a quadratic function (e.g., a sum of squares function) of the parameters to be optimized (e.g., variable residuals/error to be minimized). In some embodiments, a least squares optimization model is used in the methods of the present disclosure to minimize an objective function which measures a difference between the relative binding propensity and the observed allele frequency of an allele. In some embodiments, the optimization model is a quadratic regression. In some embodiments, the optimization model is a loess regression.

In some embodiments, the optimization model may be used to correct or adjust variables of interest (e.g., allele frequencies). In some embodiments, an optimization model and the observed allele frequency of an allele are used to determine the adjusted allele frequency of the allele. The adjusted allele frequency can further be used in downstream operations, e.g., inferring the zygosity status of the individual subject for the allele.

Further descriptions of least squares optimization can be found, e.g., in Wolberg, J. (2006). Data analysis using the method of least squares: extracting the most information from experiments. Springer Science & Business Media; Borowiak, D. (2001). Linear models, least squares and alternatives; Björck, Å. (1996). Numerical methods for least squares problems. Society for Industrial and Applied Mathematics; Luenberger. D. G. (1997) “Least-Squares Estimation”. Optimization by Vector Space Methods. New York: John Wiley & Sons. pp. 78-102; and the like.

Model Constraints

In some embodiments, the optimization model is subject to one or more constraints. Constraints limit the possible values for the variables in an optimization model. In some embodiments, the one or more constraints require that median value of the relative binding propensities for a plurality of alleles of the gene is equal to 0. In some embodiments, the one or more constraints require that median value of the relative binding propensities for a plurality of alleles of the gene is equal to 0.5. In some embodiments, the one or more constraints require that median value of the relative binding propensities for a plurality of alleles of the gene is equal to 1.

Sequencing

In some embodiments, the plurality of sequence reads was obtained by performing sequencing on nucleic acids captured by hybridization with the bait molecule. In some embodiments, the plurality of sequence reads was obtained by performing whole exome sequencing on nucleic acids captured by hybridization with the bait molecule. In some embodiments, the plurality of sequence reads was obtained by performing next-generation sequencing (NGS), whole exome sequencing, or methylation sequencing on nucleic acids captured by hybridization with the bait molecule.

In some embodiments, the methods further comprise, prior to obtaining the observed allele frequency: sequencing a plurality of polynucleotides by next-generation sequencing (NGS) in order to obtain the plurality of sequence reads, wherein the plurality of polynucleotides comprises nucleic acid(s) encoding at least a portion of the allele. NGS methods are known in the art, and are described, e.g., in Metzker, M. (2010) Nature Biotechnology Reviews 11:31-46. Platforms for next-generation sequencing include, e.g., Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, and Pacific Biosciences' PacBio RS system. NGS technologies can include one or more of steps, e.g., template preparation, sequencing and imaging, and data analysis. Methods for template preparation can include steps such as randomly breaking nucleic acids (e.g., genomic DNA) into smaller sizes and generating sequencing templates (e.g., fragment templates or mate-pair templates). The spatially separated templates can be attached or immobilized to a solid surface or support, allowing massive amounts of sequencing reactions to be performed simultaneously. Types of templates that can be used for NGS reactions include, e.g., clonally amplified templates originating from single DNA molecules, and single DNA molecule templates. Exemplary sequencing and imaging steps for NGS include, e.g., cyclic reversible termination (CRT), sequencing by ligation (SBL), single-molecule addition (pyrosequencing), and real-time sequencing. After NGS reads have been generated, they can be aligned to a known reference sequence or assembled de novo. For example, identifying genetic variations such as single-nucleotide polymorphism and structural variants in a sample (e.g., a tumor sample) can be accomplished by aligning NGS reads to a reference sequence (e.g., a wildtype sequence). Methods of sequence alignment for NGS are described e.g., in Trapnell C. and Salzberg S. L. Nature Biotech., 2009, 27:455-457. Examples of de novo assemblies are described, e.g., in Warren R et al., Bioinformatics, 2007, 23:500-501; Butler J. et al., Genome Res., 2008, 18:810-820; and Zerbino D. R. and Birney E., Genome Res., 2008, 18:821-829. Sequence alignment or assembly can be performed using read data from one or more NGS platforms, e.g., mixing Roche/454 and Illumina/Solexa read data.

In some embodiments, the methods further comprise, prior to obtaining the observed allele frequency: sequencing a plurality of polynucleotides by whole exome sequencing in order to obtain the plurality of sequence reads, wherein the plurality of polynucleotides comprises nucleic acid(s) encoding at least a portion of the allele.

In some embodiments, the methods further comprise, prior to sequencing the plurality of polynucleotides: contacting a mixture of polynucleotides with the bait molecule under conditions suitable for hybridization, wherein the mixture comprises a plurality of polynucleotides capable of hybridization with the bait molecule; and isolating a plurality of polynucleotides that hybridized with the bait molecule, wherein the isolated plurality of polynucleotides that hybridized with the bait molecule are sequenced by NGS. FIG. 1 illustrates such a hybrid capture process. Further details about this and other hybrid capture processes can be found in U.S. Pat. No. 9,340,830, the entirety of which is incorporated by reference herein. In some embodiments, the methods further comprise, prior to contacting the mixture of polynucleotides with the bait molecule: obtaining a sample from an individual, wherein the sample comprises tumor cells and/or tumor nucleic acids; and extracting the mixture of polynucleotides from the sample, wherein the mixture of polynucleotides is from the tumor cells and/or tumor nucleic acids. In some embodiments, the sample further comprises non-tumor cells.

In some embodiments, the methods comprise subjecting a plurality of polynucleotides to methylation sequencing in order to obtain the plurality of sequence reads. In some embodiments, the plurality of polynucleotides comprises nucleic acid(s) encoding at least a portion of the allele.

In some embodiments, nucleic acids are obtained from a sample, e.g., comprising tumor cells and/or tumor nucleic acids. For example, the sample can comprise tumor cell(s), circulating tumor cell(s), tumor nucleic acids (e.g., tumor circulating tumor DNA, cfDNA, or cfRNA), part or all of a tumor biopsy, fluid, cells, tissue, mRNA, genomic DNA, RNA, cell-free DNA, and/or cell-free RNA. In some embodiments, the sample is from a tumor biopsy or tumor specimen. In some embodiments, the sample further comprises non-tumor cells and/or non-tumor nucleic acids. In some embodiments, the fluid comprises blood, serum, plasma, saliva, semen, cerebral spinal fluid, amniotic fluid, peritoneal fluid, interstitial fluid, etc.

In some embodiments, the sample is or comprises biological tissue or fluid. The sample can contain compounds that are not naturally intermixed with the tissue in nature such as preservatives, anticoagulants, buffers, fixatives, nutrients, antibiotics or the like. In one embodiment, the sample is preserved as a frozen sample or as a formaldehyde- or paraformaldehyde-fixed paraffin-embedded (FFPE) tissue preparation. For example, the sample can be embedded in a matrix, e.g., an FFPE block or a frozen sample. In another embodiment, the sample is a blood or blood constituent sample. In yet another embodiment, the sample is a bone marrow aspirate sample. In another embodiment, the sample comprises cell-free DNA (cfDNA). Without wishing to be bound by theory, it is believed that in some embodiments, cfDNA is DNA from apoptosed or necrotic cells. Typically, cfDNA is bound by protein (e.g., histone) and protected by nucleases. CfDNA can be used as a biomarker, for example, for non-invasive prenatal testing (NIPT), organ transplant, cardiomyopathy, microbiome, and cancer. In another embodiment, the sample comprises circulating tumor DNA (ctDNA). Without wishing to be bound by theory, it is believed that in some embodiments, ctDNA is cfDNA with a genetic or epigenetic alteration (e.g., a somatic alteration or a methylation signature) that can discriminate it originating from a tumor cell versus a non-tumor cell. In another embodiment, the sample comprises circulating tumor cells (CTCs). Without wishing to be bound by theory, it is believed that in some embodiments, CTCs are cells shed from a primary or metastatic tumor into the circulation. In some embodiments, CTCs apoptose and are a source of ctDNA in the blood/lymph.

In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as ductal lavages or bronchoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained.

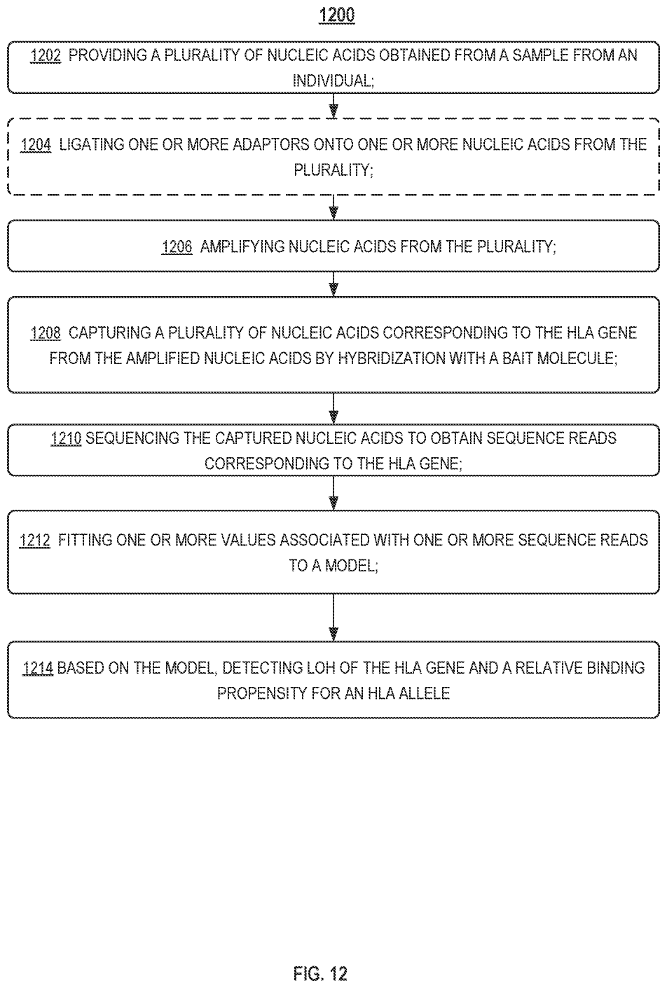

FIG. 12 illustrates an exemplary process 1200 for detecting loss-of-heterozygosity (LOH) of a human leukocyte antigen (HLA) gene, in accordance with some embodiments. Process 1200 is performed, for example, using one or more electronic devices implementing a software program. In some examples, process 1200 is performed using a client-server system, and the blocks of process 1200 are divided up in any manner between the server and a client device. In other examples, the blocks of process 1200 are divided up between the server and multiple client devices. Thus, while portions of process 1200 are described herein as being performed by particular devices of a client-server system, it will be appreciated that process 1200 is not so limited. In other examples, process 1200 is performed using only a client device or only multiple client devices. In process 1200, some blocks are, optionally, combined, the order of some blocks is, optionally, changed, and some blocks are, optionally, omitted. In some examples, additional steps may be performed in combination with the process 1200. Accordingly, the operations as illustrated (and described in greater detail below) are exemplary by nature and, as such, should not be viewed as limiting.

At block 1202, a plurality of nucleic acids obtained from a sample from an individual are provided, wherein the plurality of nucleic acids comprises nucleic acids encoding an HLA gene. Optionally, at block 1204, one or more adaptors are ligated onto one or more nucleic acids from the plurality of nucleic acids. At block 1206, nucleic acids are amplified from the plurality of nucleic acids. At block 1208, a plurality of nucleic acids corresponding to the HLA gene are captured from the amplified nucleic acids by hybridization with a bait molecule. At block 1210, an exemplary sequencer sequences the captured nucleic acids to obtain a plurality of sequence reads corresponding to the HLA gene. At block 1212, an exemplary system (e.g., one or more electronic devices) fits one or more values associated with one or more of the plurality of sequence reads to a model. At block 1214, the system detects LOH of the HLA gene and a relative binding propensity for an HLA allele of the HLA gene based on the model.

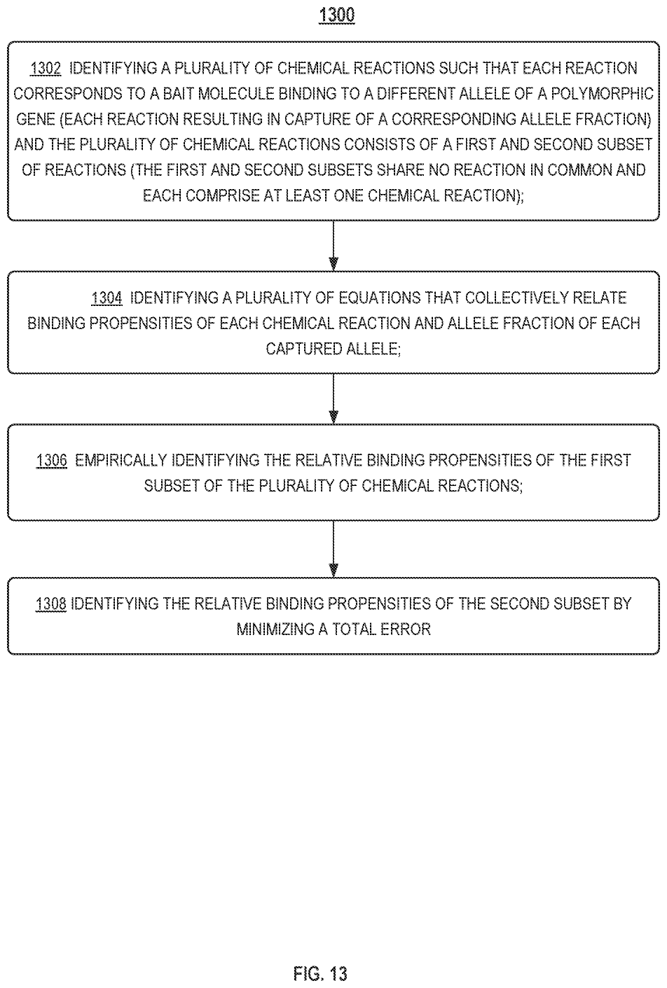

FIG. 13 illustrates an exemplary process 1300 for identifying relative binding propensities of different alleles of a polymorphic gene to a bait molecule, in accordance with some embodiments. Process 1300 is performed, for example, using one or more electronic devices implementing a software program. In some examples, process 1300 is performed using a client-server system, and the blocks of process 1300 are divided up in any manner between the server and a client device. In other examples, the blocks of process 1300 are divided up between the server and multiple client devices. Thus, while portions of process 1300 are described herein as being performed by particular devices of a client-server system, it will be appreciated that process 1300 is not so limited. In other examples, process 1300 is performed using only a client device or only multiple client devices. In process 1300, some blocks are, optionally, combined, the order of some blocks is, optionally, changed, and some blocks are, optionally, omitted. In some examples, additional steps may be performed in combination with the process 1300. Accordingly, the operations as illustrated (and described in greater detail below) are exemplary by nature and, as such, should not be viewed as limiting.

At block 1302, an exemplary system (e.g., one or more electronic devices) identifies a plurality of chemical reactions, e.g., such that each reaction corresponds to a bait molecule binding to a different allele of a polymorphic gene, and each reaction resulting in capture of a corresponding allele fraction, and the plurality of chemical reactions consists of a first subset of reactions and a second subset of reactions, in which the first and second subsets share no reaction in common and in which the first and second subsets each comprise at least one chemical reaction. At block 1304, the system identifies a plurality of equations that collectively relate binding propensities of each chemical reaction and allele fraction of each captured allele. At block 1306, the system empirically identifies the relative binding propensities of the first subset of the plurality of chemical reactions. At block 1308, the system identifies the relative binding propensities of the second subset by minimizing a total error.

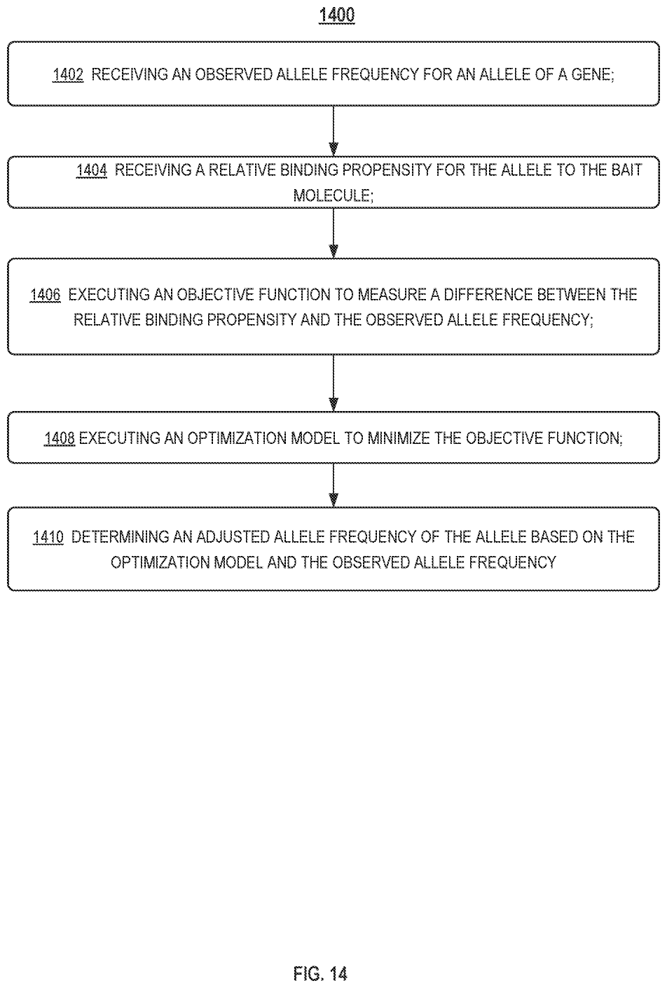

FIG. 14 illustrates an exemplary process 1400 for determining allele frequency, in accordance with some embodiments. In some embodiments, the allele frequency of one or more HLA alleles is determined, e.g., to detect LOH. Process 1400 is performed, for example, using one or more electronic devices implementing a software program. In some examples, process 1400 is performed using a client-server system, and the blocks of process 1400 are divided up in any manner between the server and a client device. In other examples, the blocks of process 1400 are divided up between the server and multiple client devices. Thus, while portions of process 1400 are described herein as being performed by particular devices of a client-server system, it will be appreciated that process 1300 is not so limited. In other examples, process 1400 is performed using only a client device or only multiple client devices. In process 1400, some blocks are, optionally, combined, the order of some blocks is, optionally, changed, and some blocks are, optionally, omitted. In some examples, additional steps may be performed in combination with the process 1400. Accordingly, the operations as illustrated (and described in greater detail below) are exemplary by nature and, as such, should not be viewed as limiting.

At block 1402, an exemplary system (e.g., one or more electronic devices) receives an observed allele frequency for an allele of a gene. In some embodiments, the observed allele frequency corresponds to frequency of nucleic acid(s) encoding at least a portion of the allele as detected among a plurality of sequence reads corresponding to the gene, and the plurality of sequence reads was obtained by sequencing nucleic acids encoding the gene or a portion thereof as captured by hybridization with a bait molecule. In some embodiments, the gene is a human HLA gene, and the alleles are human HLA alleles (e.g., as described herein). At block 1404, the system receives a relative binding propensity for the allele to the bait molecule. In some embodiments, the relative binding propensity of the allele corresponds to propensity of nucleic acid encoding at least a portion of the allele to bind the bait molecule in the presence of nucleic acids encoding portions of one or more other alleles of the gene. At block 1406, the system executes an objective function to measure a difference between the relative binding propensity and the observed allele frequency of the allele. At block 1408, the system executes an optimization model to minimize the objective function. At block 1410, the system determines an adjusted allele frequency of the allele based on the optimization model and the observed allele frequency.

Software and Devices

In some other aspects, provided herein are non-transitory computer-readable storage media. In some embodiments, the non-transitory computer-readable storage media comprise one or more programs for execution by one or more processors of a device, the one or more programs including instructions which, when executed by the one or more processors, cause the device to perform the method according to any of the embodiments described herein.

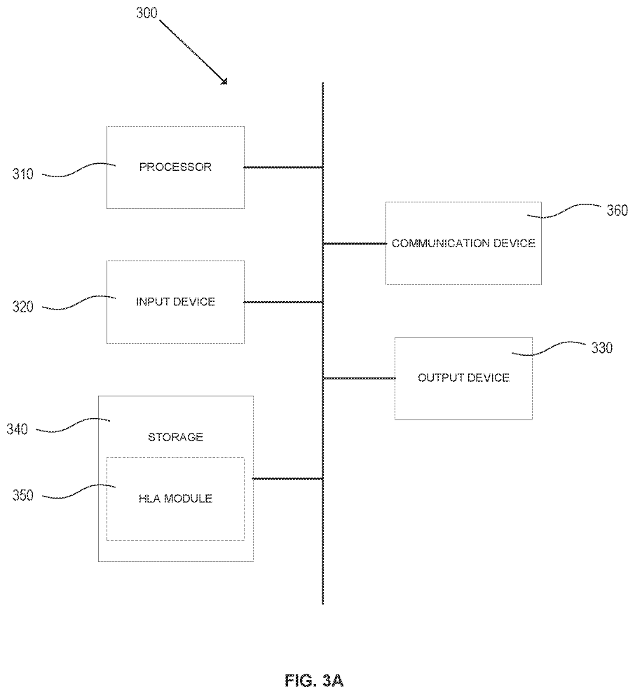

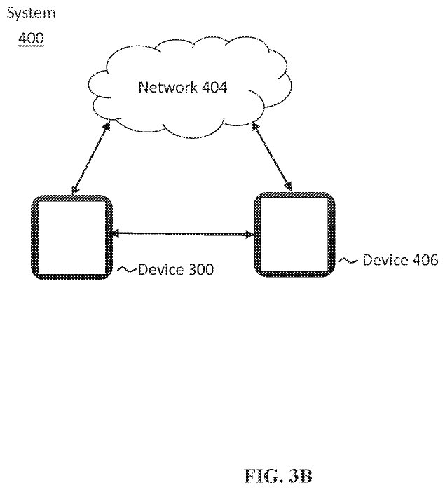

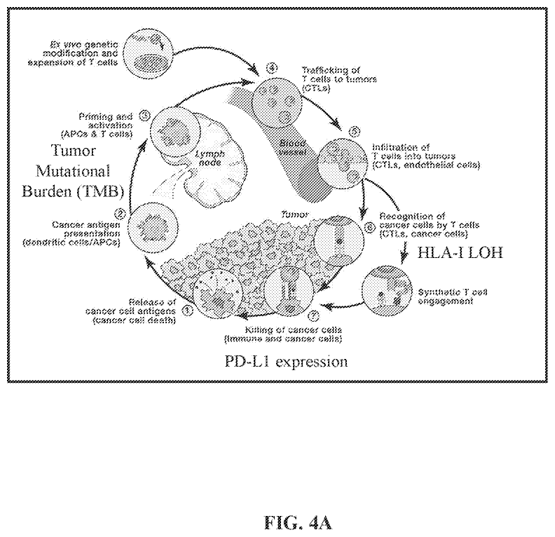

FIG. 3A illustrates an example of a computing device in accordance with one embodiment. Device 300 can be a host computer connected to a network. Device 300 can be a client computer or a server. As shown in FIG. 3A, device 300 can be any suitable type of microprocessor-based device, such as a personal computer, workstation, server or handheld computing device (portable electronic device) such as a phone or tablet. The device can include, for example, one or more of processor 310, input device 320, output device 330, storage 340, and communication device 360. Input device 320 and output device 330 can generally correspond to those described above, and can either be connectable or integrated with the computer.

Input device 320 can be any suitable device that provides input, such as a touch screen, keyboard or keypad, mouse, or voice-recognition device. Output device 330 can be any suitable device that provides output, such as a touch screen, haptics device, or speaker.

Storage 340 can be any suitable device that provides storage (e.g., an electrical, magnetic or optical memory including a RAM, cache, hard drive, or removable storage disk). Communication device 360 can include any suitable device capable of transmitting and receiving signals over a network, such as a network interface chip or device. The components of the computer can be connected in any suitable manner, such as via a wired media (e.g., a physical bus, ethernet, or any other wire transfer technology) or wirelessly (e.g., Bluetooth®, Wi-Fit, or any other wireless technology).

HLA module 350, which can be stored as executable instructions in storage 340 and executed by processor 310, can include, for example, the processes that embody the functionality of the present disclosure (e.g., as embodied in the devices as described above).

HLA module 350 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 340, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

HLA module 350 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 300 may be connected to a network (e.g., Network 404, as shown in FIG. 3B and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 300 can implement any operating system suitable for operating on the network. HLA module 350 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example.

FIG. 3B illustrates an example of a computing system in accordance with one embodiment. In System 400, Device 300 (e.g., as described above and illustrated in FIG. 3A) is connected to Network 404, which is also connected to Device 406. In some embodiments, Device 406 is a sequencer. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio RS system. Devices 300 and 406 may communicate, e.g., using suitable communication interfaces via Network 404, such as a Local Area Network (LAN), Virtual Private Network (VPN), or the Internet. In some embodiments, Network 404 can be, for example, the Internet, an intranet, a virtual private network, a cloud network, a wired network, or a wireless network. Devices 300 and 406 may communicate, in part or in whole, via wireless or hardwired communications, such as Ethernet, IEEE 802.11b wireless, or the like. Additionally, Devices 300 and 406 may communicate, e.g., using suitable communication interfaces, via a second network, such as a mobile/cellular network. Communication between Devices 300 and 406 may further include or communicate with various servers such as a mail server, mobile server, media server, telephone server, and the like. In some embodiments, Devices 300 and 406 can communicate directly (instead of, or in addition to, communicating via Network 404), e.g., via wireless or hardwired communications, such as Ethernet, IEEE 802.11b wireless, or the like.

One or all of Devices 300 and 406 generally include logic (e.g., http web server logic) or is programmed to format data, accessed from local or remote databases or other sources of data and content, for providing and/or receiving information via Network 404 according to various examples described herein.

Human Leukocyte Antigen (HLA) and Loss-of-Heterozygosity (LOH)

In some embodiments according to any of the embodiments described herein, the gene is a human leukocyte antigen (HLA) gene encoding a major histocompatibility (MHC) class I molecule. In some embodiments, the methods further comprise, after determining the adjusted allele frequency: determining that the gene has undergone loss-of-heterozygosity (LOH) based at least in part on the adjusted allele frequency. In other embodiments, the gene is ST7/RAY1, ARH1/NOEY2, TSLC1, RB, PTEN, SMAD2, SMAD4, DCC, TP53, ATM, miR-15a, miR-16-1, NAT2, BRCA1, BRCA2, hOGG1, CDH1, IGF2, CDKN1C/P57, MEN1, PRKAR1A, H19, KRAS, BAP1, PTCH1, SMO, SUFU, NOTCH1, PPP6C, LATS1, CASP8, PTPN14, ARID1A, FBXW7, M6P/IGF2R, IFN-alpha, an olfactory receptor gene, CBFA2T3, DUTT1, FHIT, APC, P16, FCMD, TSC2, miR-34, c-MPL, RUNX3, DIRAS3, NRAS, miR-9, FAM50B, PLAGL1, ER, FLT3, ZDBF2, GPR1, c-KIT, NAP1L5, GRB10, EGFR, PEG10, BRAF, MEST, JAK2, DAPK1, LTT1, WT1, NF-1, PR, c-CBL, DLK1, AKT1, SNURF, a cytochrome P450 gene (CYP), ZNF587, SOCS1, TIMP2, RUNX1, AR, CEBPA, C19MC, EMP3, ZNF331, CDKN2A, PEG3, NNAT, GNAS, or GATA5.

In yet some other aspects, provided herein are methods for detecting loss-of-heterozygosity (LOH) of a human leukocyte antigen (HLA) gene. In some embodiments, the methods comprise: a) obtaining an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule; b) obtaining a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles; c) applying an objective function to measure a difference between the relative binding propensity and the observed allele frequency of the HLA allele; d) applying an optimization model to minimize the objective function; e) determining an adjusted allele frequency of the HLA allele based on the optimization model and the observed allele frequency; and f) determining that LOH has occurred when the adjusted allele frequency of the HLA allele is less than a predetermined threshold. In some embodiments, the HLA gene is a human HLA-A, HLA-B, or HLA-C gene. In some embodiments, the plurality of sequence reads was obtained by sequencing nucleic acids obtained from a sample comprising tumor cells and/or tumor nucleic acids. In some embodiments, the sample further comprises non-tumor cells. In some embodiments, the methods are for detecting loss-of-heterozygosity (LOH) of a polymorphic gene of interest. In some embodiments, the methods comprise: a) obtaining an observed allele frequency for an allele of a gene of interest, wherein observed allele frequency corresponds to frequency of nucleic acid(s) encoding at least a portion of the allele as detected among a plurality of sequence reads corresponding to the gene, wherein the plurality of sequence reads was obtained by sequencing nucleic acids encoding the gene or a portion thereof as captured by hybridization with a bait molecule; b) obtaining a relative binding propensity for the allele to the bait molecule, wherein the relative binding propensity of the allele corresponds to propensity of nucleic acid encoding at least a portion of the allele to bind the bait molecule in the presence of nucleic acids encoding portions of one or more other alleles; c) applying an objective function to measure a difference between the relative binding propensity and the observed allele frequency of the allele; d) applying an optimization model to minimize the objective function; e) determining an adjusted allele frequency of the allele based on the optimization model and the observed allele frequency; and f) determining that LOH has occurred when the adjusted allele frequency of the allele is less than a predetermined threshold. In some embodiments, the polymorphic gene is ST7/RAY1, ARH1/NOEY2, TSLC1, RB, PTEN, SMAD2, SMAD4, DCC, TP53, ATM, miR-15a, miR-16-1, NAT2, BRCA1, BRCA2, hOGG1, CDH1, IGF2, CDKN1C/P57, MEN1, PRKAR1A, H19, KRAS, BAP1, PTCH1, SMO, SUFU, NOTCH1, PPP6C, LATS1, CASP8, PTPN14, ARID1A, FBXW7, M6P/IGF2R, IFN-alpha, an olfactory receptor gene, CBFA2T3, DUTT1, FHIT, APC, P16, FCMD, TSC2, miR-34, c-MPL, RUNX3, DIRAS3, NRAS, miR-9, FAM50B, PLAGL1, ER, FLT3, ZDBF2, GPR1, c-KIT, NAP1L5, GRB10, EGFR, PEG10, BRAF, MEST, JAK2, DAPK1, LIT1, WT1, NF-1, PR, c-CBL, DLK1, AKT1, SNURF, a cytochrome P450 gene (CYP), ZNF587, SOCS1, TIMP2, RUNX1, AR, CEBPA, C19MC, EMP3, ZNF331, CDKN2A, PEG3, NNAT, GNAS, or GATA5.

In yet some other aspects, any of the methods of the present disclosure further comprise measuring TMB, e.g., in a sample of the present disclosure comprising tumor cells and/or tumor nucleic acids. In some embodiments, the methods comprise determining LOH and assessing TMB, e.g., in a sample of the present disclosure. As demonstrated herein, HLA LOH and high TMB (and optionally intact HLA gene(s)) may be predictive of increased overall survival, increased probability of greater survival, and/or increased likelihood of response to ICI therapy, e.g., as compared to HLA LOH without high TMB. In some embodiments, high TMB refers to a TMB of greater than or equal to 10 mutations/Mb or greater than or equal to 13 mutations/Mb. In some embodiments, TMB is obtained from a plurality of sequence reads, e.g., a plurality of sequence reads obtained by sequencing nucleic acids at least a portion of a genome (such as from an enriched or unenriched sample). In some embodiments, TMB is determined based on a number of non-driver somatic coding mutations per megabase of genome sequenced.

In some embodiments, any of the methods of the present disclosure comprise acquiring knowledge of LOH of the HLA gene (e.g., in a sample obtained from an individual) and acquiring knowledge of TMB (e.g., in a sample obtained from an individual). In some embodiments, any of the methods of the present disclosure comprise detecting LOH of the HLA gene (e.g., in a sample obtained from an individual) and acquiring knowledge of TMB (e.g., in a sample obtained from an individual). In some embodiments, any of the methods of the present disclosure comprise acquiring knowledge of LOH of the HLA gene (e.g., in a sample obtained from an individual) and detecting or determining TMB (e.g., in a sample obtained from an individual). In some embodiments, any of the methods of the present disclosure comprise detecting LOH of the HLA gene (e.g., in a sample obtained from an individual) and detecting or determining TMB (e.g., in a sample obtained from an individual). In some embodiments, the samples used to detect/determine LOH and TMB are the same. In some embodiments, the samples used to detect/determine LOH and TMB are different.

Treatments and Therapies

In still some other aspects, provided herein are methods of identifying an individual having cancer where loss-of-heterozygosity (LOH) of a human leukocyte antigen (HLA) gene indicates a propensity of the individual with a particular type of disease to respond to a particular treatment. In some embodiments, the methods comprise: detecting LOH of the HLA gene in a sample from the individual, wherein LOH of the HLA gene is detected according to the method according to any of the embodiments described herein. In some embodiments, LOH of the HLA gene in the sample indicates that the individual is not likely to benefit from a treatment comprising an ICI. In some embodiments, detecting lack of LOH of the HLA gene in the sample indicates that the individual is likely to benefit from a treatment comprising an ICI. In some embodiments, the methods further comprise: detecting a tumor mutation burden (TMB) in a sample obtained from the individual. In some embodiments, the methods further comprise: acquiring knowledge of a high tumor mutation burden (TMB) in a sample obtained from the individual. In some embodiments, LOH of the HLA gene and high TMB indicate that the individual is likely to benefit from a treatment comprising an ICI. In some embodiments, LOH of the HLA gene and low TMB, or LOH of the HLA gene without high TMB, indicate that the individual is not likely to benefit from a treatment comprising an ICI.

In still some other aspects, provided herein are methods of selecting a therapy for an individual having cancer. In some embodiments, the methods comprise: detecting loss-of-heterozygosity (LOH) of a human leukocyte antigen (HLA) gene in a sample from the individual, wherein LOH of the HLA gene is detected according to the method according to any of the embodiments described herein. In some embodiments, LOH of the HLA gene in the sample indicates that the individual is not likely to benefit from a treatment comprising an ICI. In some embodiments, detecting lack of LOH of the HLA gene in the sample indicates that the individual is likely to benefit from a treatment comprising an ICI. In some embodiments, the methods further comprise: detecting a tumor mutation burden (TMB) in a sample obtained from the individual. In some embodiments, the methods further comprise: acquiring knowledge of a high tumor mutation burden (TMB) in a sample obtained from the individual. In some embodiments, LOH of the HLA gene and high TMB indicate that the individual is likely to benefit from a treatment comprising an ICI. In some embodiments, LOH of the HLA gene and low TMB, or LOH of the HLA gene without high TMB, indicate that the individual is not likely to benefit from a treatment comprising an ICI.

In still some other aspects, provided herein are methods of identifying one or more treatment options for an individual having cancer. In some embodiments, the methods comprise: (a) acquiring knowledge of loss-of-heterozygosity (LOH) of a human leukocyte antigen (HLA) gene in a sample from the individual, wherein LOH of the HLA gene is detected according to the method according to any of the embodiments described herein; and (b) generating a report comprising one or more treatment options identified for the individual based at least in part on said knowledge. In some embodiments, LOH of the HLA gene in the sample indicates that the individual is not likely to benefit from a treatment comprising an ICI. In some embodiments, the one or more treatment options do not include treatment comprising an ICI. In some embodiments, the methods comprise: (a) acquiring knowledge of lack of loss-of-heterozygosity (LOH) of a human leukocyte antigen (HLA) gene in a sample from the individual, wherein lack of LOH of the HLA gene is detected according to the method according to any of the embodiments described herein; and (b) generating a report comprising one or more treatment options identified for the individual based at least in part on said knowledge. In some embodiments, lack of LOH of the HLA gene in the sample indicates that the individual is likely to benefit from a treatment comprising an ICI. In some embodiments, the methods further comprise: detecting a tumor mutation burden (TMB) in a sample obtained from the individual. In some embodiments, LOH of the HLA gene and high TMB indicate that the individual is likely to benefit from a treatment comprising an ICI. In some embodiments, LOH of the HLA gene and low TMB, or LOH of the HLA gene without high TMB, indicate that the individual is not likely to benefit from a treatment comprising an ICI. In some embodiments, the methods further comprise acquiring knowledge of high TMB in a sample from the individual, and the one or more treatment options include treatment comprising an ICI.

In still some other aspects, provided herein are methods of selecting treatment for an individual having cancer. In some embodiments, the methods comprise acquiring knowledge of loss-of-heterozygosity (LOH) of a human leukocyte antigen (HLA) gene in a sample from an individual having cancer, wherein LOH of the HLA gene is detected according to the method according to any of the embodiments described herein. In some embodiments, responsive to the acquisition of said knowledge: (i) the individual is classified as a candidate not to receive treatment with an immune checkpoint inhibitor (ICI); (ii) the individual is identified as not likely to respond to a treatment that comprises an immune checkpoint inhibitor (ICI); and/or (iii) the individual is classified as a candidate to receive a treatment other than an immune checkpoint inhibitor (ICI). In some embodiments, the methods comprise acquiring knowledge of lack of loss-of-heterozygosity (LOH) of a human leukocyte antigen (HLA) gene in a sample from an individual having cancer, wherein lack of LOH of the HLA gene is detected according to the method according to any of the embodiments described herein. In some embodiments, responsive to the acquisition of said knowledge: (i) the individual is classified as a candidate to receive treatment with an immune checkpoint inhibitor (ICI); and/or (ii) the individual is identified as likely to respond to a treatment that comprises an immune checkpoint inhibitor (ICI). In some embodiments, the methods comprise: acquiring knowledge of LOH of a human leukocyte antigen (HLA) gene in a sample obtained from the individual and acquiring knowledge of a high tumor mutation burden (TMB) in a sample obtained from the individual. In some embodiments, responsive to the acquisition of said knowledge: (i) the individual is classified as a candidate to receive treatment with an immune checkpoint inhibitor (ICI); and/or (ii) the individual is identified as likely to respond to a treatment that comprises an immune checkpoint inhibitor (ICI).

In still some other aspects, provided herein are methods of predicting survival of an individual having cancer treated with an immune checkpoint inhibitor (ICI). In some embodiments, the methods comprise acquiring knowledge of loss-of-heterozygosity (LOH) of a human leukocyte antigen (HLA) gene in a sample from the individual, wherein LOH of the HLA gene is detected according to the method according to any of the embodiments described herein. In some embodiments, responsive to the acquisition of said knowledge, the individual is predicted to have shorter survival after treatment with the ICI, as compared to survival of an individual treated with the ICI whose cancer does not exhibit LOH of the HLA gene. In some embodiments, the methods comprise acquiring knowledge of lack of loss-of-heterozygosity (LOH) of a human leukocyte antigen (HLA) gene in a sample from the individual, wherein lack of LOH of the HLA gene is detected according to the method according to any of the embodiments described herein. In some embodiments, responsive to the acquisition of said knowledge, the individual is predicted to have longer survival after treatment with the ICI, as compared to survival of an individual treated with the ICI whose cancer exhibits LOH of the HLA gene. In some embodiments, the methods comprise acquiring knowledge of loss-of-heterozygosity (LOH) of a human leukocyte antigen (HLA) gene in a sample from the individual and acquiring knowledge of a high tumor mutation burden (TMB) in a sample obtained from the individual, wherein LOH of the HLA gene is detected according to the method according to any of the embodiments described herein. In some embodiments, responsive to the acquisition of said knowledge, the individual is predicted to have longer survival after treatment with the ICI, as compared to survival of an individual treated with the ICI whose cancer has LOH of the HLA gene without a high TMB.

In some embodiments according to any of the embodiments described herein, LOH of the HLA gene is determined by: a) obtaining an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule; b) obtaining a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles; c) determining an objective function that measures a difference between the relative binding propensity and the observed allele frequency of the HLA allele; d) determining an optimization model configured to minimize the objective function; e) determining an adjusted allele frequency of the HLA allele based on the optimization model and the observed allele frequency; and f) determining that LOH has occurred when the adjusted allele frequency of the HLA allele is less than a predetermined threshold.

In still some other aspects, provided herein are methods of treating or delaying progression of cancer. In some embodiments, the methods comprise: (1) detecting loss-of-heterozygosity (LOH) of a human leukocyte antigen (HLA) gene in a sample obtained from an individual, wherein LOH of the HLA gene is detected by: a) obtaining an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule; b) obtaining a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles; c) applying an objective function to measure a difference between the relative binding propensity and the observed allele frequency of the HLA allele; d) applying an optimization model to minimize the objective function; e) determining an adjusted allele frequency of the HLA allele based on the optimization model and the observed allele frequency; and f) determining that LOH has occurred when the adjusted allele frequency of the HLA allele is less than a predetermined threshold; and (2) based at least in part on detection of LOH of the HLA gene, administering an effective amount of a treatment other than an immune checkpoint inhibitor (ICI) to the individual. In some embodiments, the methods comprise: (1) detecting lack of loss-of-heterozygosity (LOH) of a human leukocyte antigen (HLA) gene in a sample obtained from an individual, wherein lack of LOH of the HLA gene is detected by: a) obtaining an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule; b) obtaining a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles; c) applying an objective function to measure a difference between the relative binding propensity and the observed allele frequency of the HLA allele; d) applying an optimization model to minimize the objective function; e) determining an adjusted allele frequency of the HLA allele based on the optimization model and the observed allele frequency; and f) determining that LOH has not occurred when the adjusted allele frequency of the HLA allele is greater than a predetermined threshold; and (2) based at least in part on detection of lack of LOH of the HLA gene, administering an effective amount of an immune checkpoint inhibitor (ICI) to the individual.

Certain aspects of the present disclosure relate to immune checkpoint inhibitors (ICIs). As is known in the art, a checkpoint inhibitor targets at least one immune checkpoint protein to alter the regulation of an immune response. Immune checkpoint proteins include, e.g., CTLA4, PD-L1, PD-1, PD-L2, VISTA, B7-H2, B7-H3, B7-H4, B7-H6, 2B4, ICOS, HVEM, CEACAM, LAIR1, CD80, CD86, CD276, VTCN1, MHC class I, MHC class II, GALS, adenosine, TGFR, CSF1R, MICA/B, arginase, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, LAG-3, BTLA, IDO, OX40, and A2aR. In some embodiments, molecules involved in regulating immune checkpoints include, but are not limited to: PD-1 (CD279), PD-L1 (B7-H1, CD274), PD-L2 (B7-CD, CD273), CTLA-4 (CD152), HVEM, BTLA (CD272), a killer-cell immunoglobulin-like receptor (KIR), LAG-3 (CD223), TIM-3 (HAVCR2), CEACAM, CEACAM-1, CEACAM-3, CEACAM-5, GAL9, VISTA (PD-1H), TIGIT, LAIR1, CD160, 2B4, TGFRbeta, A2AR, GITR (CD357), CD80 (B7-1), CD86 (B7-2), CD276 (B7-H3), VTCNI (B7-H4), MHC class I, MHC class II, GALS, adenosine, TGFR, B7-H1, OX40 (CD134), CD94 (KLRD1), CD137 (4-1BB), CD137L (4-1BBL), CD40, IDO, CSF1R, CD40L, CD47, CD70 (CD27L), CD226, HHLA2, ICOS (CD278), ICOSL (CD275), LIGHT (TNFSF14, CD258), NKG2a, NKG2d, OX40L (CD134L), PVR (NECL5, CD155), SIRPa, MICA/B, and/or arginase. In some embodiments, an immune checkpoint inhibitor (i.e., a checkpoint inhibitor) decreases the activity of a checkpoint protein that negatively regulates immune cell function, e.g., in order to enhance T cell activation and/or an anti-cancer immune response. In other embodiments, a checkpoint inhibitor increases the activity of a checkpoint protein that positively regulates immune cell function, e.g., in order to enhance T cell activation and/or an anti-cancer immune response. In some embodiments, the checkpoint inhibitor is an antibody. Examples of checkpoint inhibitors include, without limitation, a PD-1 axis binding antagonist, a PD-L1 axis binding antagonist (e.g., an anti-PD-L1 antibody, e.g., atezolizumab (MPDL3280A)), an antagonist directed against a co-inhibitory molecule (e.g., a CTLA4 antagonist (e.g., an anti-CTLA4 antibody), a TIM-3 antagonist (e.g., an anti-TIM-3 antibody), or a LAG-3 antagonist (e.g., an anti-LAG-3 antibody)), or any combination thereof. In some embodiments, the immune checkpoint inhibitors comprise drugs such as small molecules, recombinant forms of ligand or receptors, or antibodies, such as human antibodies (see, e.g., International Patent Publication WO2015016718; Pardoll, Nat Rev Cancer, 12(4): 252-64, 2012; both incorporated herein by reference). In some embodiments, known inhibitors of immune checkpoint proteins or analogs thereof may be used, in particular chimerized, humanized or human forms of antibodies may be used.

In some embodiments according to any of the embodiments described herein, the ICI comprises a PD-1 antagonist/inhibitor or a PD-L1 antagonist/inhibitor.

In some embodiments, the checkpoint inhibitor is a PD-L1 axis binding antagonist, e.g., a PD-1 binding antagonist, a PD-L1 binding antagonist, or a PD-L2 binding antagonist. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. PD-L1 (programmed death ligand 1) is also referred to in the art as “programmed cell death 1 ligand 1,” “PDCD1 LG1,” “CD274,” “B7-H,” and “PDL1.” An exemplary human PD-L1 is shown in UniProtKB/Swiss-Prot Accession No. Q9NZQ7.1. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some instances, PD-1, PD-L1, and PD-L2 are human PD-1, PD-L1 and PD-L2.

In some instances, the PD-1 binding antagonist/inhibitor is a molecule that inhibits the binding of PD-1 to its ligand binding partners. In a specific embodiment, the PD-1 ligand binding partners are PD-L1 and/or PD-L2. In another instance, a PD-L1 binding antagonist/inhibitor is a molecule that inhibits the binding of PD-L1 to its binding ligands. In a specific embodiment, PD-L1 binding partners are PD-1 and/or B7-1. In another instance, the PD-L2 binding antagonist is a molecule that inhibits the binding of PD-L2 to its ligand binding partners. In a specific embodiment, the PD-L2 binding ligand partner is PD-1. The antagonist may be an antibody, an antigen binding fragment thereof, an immunoadhesin, a fusion protein, or an oligopeptide. In some embodiments, the PD-1 binding antagonist is a small molecule, a nucleic acid, a polypeptide (e.g., antibody), a carbohydrate, a lipid, a metal, or a toxin.

In some instances, the PD-1 binding antagonist is an anti-PD-1 antibody (e.g., a human antibody, a humanized antibody, or a chimeric antibody), for example, as described below. In some instances, the anti-PD-1 antibody is MDX-1 106 (nivolumab), MK-3475 (pembrolizumab, Keytruda®), MEDI-0680 (AMP-514), PDR001, REGN2810, MGA-012, JNJ-63723283, BI 754091, or BGB-108. In other instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some instances, the PD-1 binding antagonist is AMP-224. Other examples of anti-PD-1 antibodies include, but are not limited to, MEDI-0680 (AMP-514; AstraZeneca), PDR001 (CAS Registry No. 1859072-53-9; Novartis), REGN2810 (LIBTAYO® or cemiplimab-rwlc; Regeneron), BGB-108 (BeiGene), BGB-A317 (BeiGene), BI 754091, JS-001 (Shanghai Junshi), STI-A1110 (Sorrento), INCSHR-1210 (Incyte), PF-06801591 (Pfizer), TSR-042 (also known as ANB011; Tesaro/AnaptysBio), AM0001 (ARMO Biosciences), ENUM 244C8 (Enumeral Biomedical Holdings), or ENUM 388D4 (Enumeral Biomedical Holdings). In some embodiments, the PD-1 axis binding antagonist comprises tislelizumab (BGB-A317), BGB-108, STI-A1110, AM0001, BI 754091, sintilimab (IBI308), cetrelimab (JNJ-63723283), toripalimab (JS-001), camrelizumab (SHR-1210, INCSHR-1210, HR-301210), MEDI-0680 (AMP-514), MGA-012 (INCMGA 0012), nivolumab (BMS-936558, MDX1106, ONO-4538), spartalizumab (PDR001), pembrolizumab (MK-3475, SCH 900475, Keytruda®), PF-06801591, cemiplimab (REGN-2810, REGEN2810), dostarlimab (TSR-042, ANB011), FITC-YT-16 (PD-1 binding peptide), APL-501 or CBT-501 or genolimzumab (GB-226), AB-122, AK105, AMG 404, BCD-100, F520, HLX10, HX008, JTX-4014, LZM009, Sym021, PSB205, AMP-224 (fusion protein targeting PD-1), CX-188 (PD-1 probody), AGEN-2034, GLS-010, budigalimab (ABBV-181), AK-103, BAT-1306, CS-1003, AM-0001, TILT-123, BH-2922, BH-2941, BH-2950, ENUM-244C8, ENUM-388D4, HAB-21, H EISCOI 11-003, IKT-202, MCLA-134, MT-17000, PEGMP-7, PRS-332, RXI-762, STI-1110, VXM-10, XmAb-23104, AK-112, HLX-20, SSI-361, AT-16201, SNA-01, AB122, PD1-PIK, PF-06936308, RG-7769, CAB PD-1Abs, AK-123, MEDI-3387, MEDI-5771, 4H1128Z-E27, REMD-288, SG-001, BY-24.3, CB-201, IBT-319, ONCR-177, Max-1, CS-4100, JBI-426, CCC-0701, or CCX-4503, or derivatives thereof.

In some embodiments, the PD-L1 binding antagonist is a small molecule that inhibits PD-1. In some embodiments, the PD-L1 binding antagonist is a small molecule that inhibits PD-L1. In some embodiments, the PD-L1 binding antagonist is a small molecule that inhibits PD-L1 and VISTA or PD-L1 and TIM3. In some embodiments, the PD-L1 binding antagonist is CA-170 (also known as AUPM-170). In some embodiments, the PD-L1 binding antagonist is an anti-PD-L1 antibody. In some embodiments, the anti-PD-L1 antibody can bind to a human PD-L1, for example a human PD-L1 as shown in UniProtKB/Swiss-Prot Accession No. Q9NZQ7.1, or a variant thereof. In some embodiments, the PD-L1 binding antagonist is a small molecule, a nucleic acid, a polypeptide (e.g., antibody), a carbohydrate, a lipid, a metal, or a toxin.

In some instances, the PD-L1 binding antagonist is an anti-PD-L1 antibody, for example, as described below. In some instances, the anti-PD-L1 antibody is capable of inhibiting the binding between PD-L1 and PD-1, and/or between PD-L1 and B7-1. In some instances, the anti-PD-L1 antibody is a monoclonal antibody. In some instances, the anti-PD-L1 antibody is an antibody fragment selected from a Fab, Fab′-SH, Fv, scFv, or (Fab′)2 fragment. In some instances, the anti-PD-L1 antibody is a humanized antibody. In some instances, the anti-PD-L1 antibody is a human antibody. In some instances, the anti-PD-L1 antibody is selected from YW243.55.S70, MPDL3280A (atezolizumab), MDX-1 105, MEDI4736 (durvalumab), or MSB0001718C (avelumab). In some embodiments, the PD-L1 axis binding antagonist comprises atezolizumab, avelumab, durvalumab (imfinzi), BGB-A333, SHR-1316 (HTI-1088), CK-301, BMS-936559, envafolimab (KN035, ASC22), CS1001, MDX-1105 (BMS-936559), LY3300054, ST1-A1014, FAZ053, CX-072, INCB086550, GNS-1480, CA-170, CK-301, M-7824, HTI-1088 (HTI-131, SHR-1316), MSB-2311, AK-106, AVA-004, BBI-801, CA-327, CBA-0710, CBT-502, FPT-155, IKT-201, IKT-703, 10-103, JS-003, KD-033, KY-1003, MCLA-145, MT-5050, SNA-02, BCD-135, APL-502 (CBT-402 or TQB2450), IMC-001, KD-045, INBRX-105, KN-046, IMC-2102, IMC-2101, KD-005, IMM-2502, 89Zr-CX-072, 89Zr-DFO-6E11, KY-1055, MEDI-1109, MT-5594, SL-279252, DSP-106, Gensci-047, REMD-290, N-809, PRS-344, FS-222, GEN-1046, BH-29xx, or FS-118, or a derivative thereof.

In some embodiments, the checkpoint inhibitor is an antagonist/inhibitor of CTLA4. In some embodiments, the checkpoint inhibitor is a small molecule antagonist of CTLA4. In some embodiments, the checkpoint inhibitor is an anti-CTLA4 antibody. CTLA4 is part of the CD28-B7 immunoglobulin superfamily of immune checkpoint molecules that acts to negatively regulate T cell activation, particularly CD28-dependent T cell responses. CTLA4 competes for binding to common ligands with CD28, such as CD80 (B7-1) and CD86 (B7-2), and binds to these ligands with higher affinity than CD28. Blocking CTLA4 activity (e.g., using an anti-CTLA4 antibody) is thought to enhance CD28-mediated costimulation (leading to increased T cell activation/priming), affect T cell development, and/or deplete Tregs (such as intratumoral Tregs). In some embodiments, the CTLA4 antagonist is a small molecule, a nucleic acid, a polypeptide (e.g., antibody), a carbohydrate, a lipid, a metal, or a toxin. In some embodiments, the CTLA-4 inhibitor comprises ipilimumab (IBI310, BMS-734016, MDX010, MDX-CTLA4, MED14736), tremelimumab (CP-675, CP-675,206), APL-509, AGEN1884, CS1002, AGEN1181, Abatacept (Orencia, BMS-188667, RG2077), BCD-145, ONC-392, ADU-1604, REGN4659, ADG116, KN044, KN046, or a derivative thereof.

In some embodiments, the anti-PD-1 antibody or antibody fragment is MDX-1106 (nivolumab), MK-3475 (pembrolizumab, Keytruda®), MEDI-0680 (AMP-514), PDR001, REGN2810, MGA-012, JNJ-63723283, BI 754091, BGB-108, BGB-A317, JS-001, STI-A1110, INCSHR-1210, PF-06801591, TSR-042, AM0001, ENUM 244C8, or ENUM 388D4. In some embodiments, the PD-1 binding antagonist is an anti-PD-1 immunoadhesin. In some embodiments, the anti-PD-1 immunoadhesin is AMP-224. In some embodiments, the anti-PD-L1 antibody or antibody fragment is YW243.55.S70, MPDL3280A (atezolizumab), MDX-1105, MED14736 (durvalumab), MSB0010718C (avelumab), LY3300054, STI-A1014, KN035, FAZ053, or CX-072.

In some embodiments, the immune checkpoint inhibitor comprises a LAG-3 inhibitor (e.g., an antibody, an antibody conjugate, or an antigen-binding fragment thereof). In some embodiments, the LAG-3 inhibitor comprises a small molecule, a nucleic acid, a polypeptide (e.g., an antibody), a carbohydrate, a lipid, a metal, or a toxin. In some embodiments, the LAG-3 inhibitor comprises a small molecule. In some embodiments, the LAG-3 inhibitor comprises a LAG-3 binding agent. In some embodiments, the LAG-3 inhibitor comprises an antibody, an antibody conjugate, or an antigen-binding fragment thereof. In some embodiments, the LAG-3 inhibitor comprises eftilagimod alpha (IMP321, IMP-321, EDDP-202, EOC-202), relatlimab (BMS-986016), GSK2831781 (IMP-731), LAG525 (IMP70l), TSR-033, EVIP321 (soluble LAG-3 protein), BI 754111, IMP761, REGN3767, MK-4280, MGD-013, XmAb22841, INCAGN-2385, ENUM-006, AVA-017, AM-0003, iOnctura anti-LAG-3 antibody, Arcus Biosciences LAG-3 antibody, Sym022, a derivative thereof, or an antibody that competes with any of the preceding.

In some embodiments, the anti-cancer therapy comprises an immunoregulatory molecule or a cytokine. In some embodiments, the methods provided herein comprise administering to the individual an immunoregulatory molecule or a cytokine, e.g., in combination with another anti-cancer therapy. An immunoregulatory profile is required to trigger an efficient immune response and balance the immunity in a subject. Examples of suitable immunoregulatory cytokines include, but are not limited to, interferons (e.g., IFNα, IFNβ and IFNγ), interleukins (e.g., IL-1, IL-2, IL-3, IL-4, IL-5, IL-6, IL-7, IL-8, IL-9, IL-10, IL-12 and IL-20), tumor necrosis factors (e.g., TNFα and TNFβ), erythropoietin (EPO), FLT-3 ligand, gip10, TCA-3, MCP-1, MIF, MIP-1α, MIP-1β, Rantes, macrophage colony stimulating factor (M-CSF), granulocyte colony stimulating factor (G-CSF), or granulocyte-macrophage colony stimulating factor (GM-CSF), as well as functional fragments thereof. In some embodiments, any immunomodulatory chemokine that binds to a chemokine receptor, i.e., a CXC, CC, C, or CX3C chemokine receptor, can be used in the context of the present disclosure. Examples of chemokines include, but are not limited to, MIP-3α (Lax), MIP-3β, Hcc-1, MPIF-1, MPIF-2, MCP-2, MCP-3, MCP-4, MCP-5, Eotaxin, Tarc, Elc, 1309, IL-8, GCP-2 Groa, Gro-P, Nap-2, Ena-78, Ip-10, MIG, I-Tac, SDF-1, or BCA-1 (BIc), as well as functional fragments thereof. In some embodiments, the immunoregulatory molecule is included with any of the treatments provided herein.

In some embodiments, the immune checkpoint inhibitor is monovalent and/or monospecific. In some embodiments, the immune checkpoint inhibitor is multivalent and/or multispecific.

In some embodiments, the methods comprise administering a second therapeutic agent. In some embodiments, the second agent is an agent other than an ICI (e.g., as described infra), or a second ICI (e.g., as described supra).

In some embodiments, the methods comprise administering an agent other than an ICI. In some embodiments, the agent comprises a chemotherapeutic agent, an anti-hormonal agent, an antimetabolite chemotherapeutic agent, a kinase inhibitor, a peptide, a gene therapy, a vaccine, a platinum-based chemotherapeutic agent, an immunotherapy, or an antibody.

In some embodiments, the anti-cancer therapy comprises a chemotherapy. In some embodiments, the methods provided herein comprise administering to the individual a chemotherapy, e.g., in combination with another anti-cancer therapy. Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antibiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenishers such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylomithine (DMFO); retinoids, such as retinoic acid; capecitabine; carboplatin, procarbazine, plicomycin, gemcitabine, navelbine, famesyl-protein tansferase inhibitors, transplatinum, and pharmaceutically acceptable salts, acids, or derivatives of any of the above.

Some non-limiting examples of chemotherapeutic drugs which can be combined with anti-cancer therapies of the present disclosure are carboplatin (Paraplatin), cisplatin (Platinol, Platinol-AQ), cyclophosphamide (Cytoxan, Neosar), docetaxel (Taxotere), doxorubicin (Adriamycin), erlotinib (Tarceva), etoposide (VePesid), fluorouracil (5-FU), gemcitabine (Gemzar), imatinib mesylate (Gleevec), irinotecan (Camptosar), methotrexate (Folex, Mexate, Amethopterin), paclitaxel (Taxol, Abraxane), sorafinib (Nexavar), sunitinib (Sutent), topotecan (Hycamtin), vincristine (Oncovin, Vincasar PFS), and vinblastine (Velban).

In some embodiments, the anti-cancer therapy comprises a kinase inhibitor. In some embodiments, the methods provided herein comprise administering to the individual a kinase inhibitor, e.g., in combination with another anti-cancer therapy. Examples of kinase inhibitors include those that target one or more receptor tyrosine kinases, e.g., BCR-ABL, B-Raf, EGFR, HER-2/ErbB2, IGF-IR, PDGFR-a, PDGFR-β, cKit, Flt-4, Flt3, FGFR1, FGFR3, FGFR4, CSF1R, c-Met, RON, c-Ret, or ALK; one or more cytoplasmic tyrosine kinases, e.g., c-SRC, c-YES, Abl, or JAK-2; one or more serine/threonine kinases, e.g., ATM, Aurora A & B, CDKs, mTOR, PKCi, PLKs, b-Raf, S6K, or STK11/LKB1; or one or more lipid kinases, e.g., PI3K or SKI. Small molecule kinase inhibitors include PHA-739358, nilotinib, dasatinib, PD166326, NSC 743411, lapatinib (GW-572016), canertinib (CI-1033), semaxinib (SU5416), vatalanib (PTK787/ZK222584), sutent (SU1 1248), sorafenib (BAY 43-9006), or leflunomide (SU101). Additional non-limiting examples of tyrosine kinase inhibitors include imatinib (Gleevec/Glivec) and gefitinib (Iressa).

In some embodiments, the anti-cancer therapy comprises an anti-angiogenic agent. In some embodiments, the methods provided herein comprise administering to the individual an anti-angiogenic agent, e.g., in combination with another anti-cancer therapy. Angiogenesis inhibitors prevent the extensive growth of blood vessels (angiogenesis) that tumors require to survive. Non-limiting examples of angiogenesis-mediating molecules or angiogenesis inhibitors which may be used in the methods of the present disclosure include soluble VEGF (for example: VEGF isoforms, e.g., VEGF121 and VEGF165; VEGF receptors, e.g., VEGFR1, VEGFR2; and co-receptors, e.g., Neuropilin-1 and Neuropilin-2), NRP-1, angiopoietin 2, TSP-1 and TSP-2, angiostatin and related molecules, endostatin, vasostatin, calreticulin, platelet factor-4, TIMP and CDAI, Meth-1 and Meth-2, IFNα, IFN-0 and IFN-γ, CXCL10, IL-4, IL-12 and IL-18, prothrombin (kringle domain-2), antithrombin III fragment, prolactin, VEGI, SPARC, osteopontin, maspin, canstatin, proliferin-related protein, restin and drugs such as bevacizumab, itraconazole, carboxyamidotriazole, TNP-470, CM101, IFN-a platelet factor-4, suramin, SU5416, thrombospondin, VEGFR antagonists, angiostatic steroids and heparin, cartilage-derived angiogenesis inhibitory factor, matrix metalloproteinase inhibitors, 2-methoxyestradiol, tecogalan, tetrathiomolybdate, thalidomide, thrombospondin, prolactina ν β3 inhibitors, linomide, or tasquinimod. In some embodiments, known therapeutic candidates that may be used according to the methods of the disclosure include naturally occurring angiogenic inhibitors, including without limitation, angiostatin, endostatin, or platelet factor-4. In another embodiment, therapeutic candidates that may be used according to the methods of the disclosure include, without limitation, specific inhibitors of endothelial cell growth, such as TNP-470, thalidomide, and interleukin-12. Still other anti-angiogenic agents that may be used according to the methods of the disclosure include those that neutralize angiogenic molecules, including without limitation, antibodies to fibroblast growth factor, antibodies to vascular endothelial growth factor, antibodies to platelet derived growth factor, or antibodies or other types of inhibitors of the receptors of EGF, VEGF or PDGF. In some embodiments, anti-angiogenic agents that may be used according to the methods of the disclosure include, without limitation, suramin and its analogs, and tecogalan. In other embodiments, anti-angiogenic agents that may be used according to the methods of the disclosure include, without limitation, agents that neutralize receptors for angiogenic factors or agents that interfere with vascular basement membrane and extracellular matrix, including, without limitation, metalloprotease inhibitors and angiostatic steroids. Another group of anti-angiogenic compounds that may be used according to the methods of the disclosure includes, without limitation, anti-adhesion molecules, such as antibodies to integrin alpha v beta 3. Still other anti-angiogenic compounds or compositions that may be used according to the methods of the disclosure include, without limitation, kinase inhibitors, thalidomide, itraconazole, carboxyamidotriazole, CM101, IFN-α, IL-12, SU5416, thrombospondin, cartilage-derived angiogenesis inhibitory factor, 2-methoxyestradiol, tetrathiomolybdate, thrombospondin, prolactin, and linomide. In one particular embodiment, the anti-angiogenic compound that may be used according to the methods of the disclosure is an antibody to VEGF, such as Avastin®/bevacizumab (Genentech).

In some embodiments, the anti-cancer therapy comprises an anti-DNA repair therapy. In some embodiments, the methods provided herein comprise administering to the individual an anti-DNA repair therapy, e.g., in combination with another anti-cancer therapy. In some embodiments, the anti-DNA repair therapy is a PARP inhibitor (e.g., talazoparib, rucaparib, olaparib), a RAD51 inhibitor (e.g., RI-1), or an inhibitor of a DNA damage response kinase, e.g., CHCK1 (e.g., AZD7762), ATM (e.g., KU-55933, KU-60019, NU7026, or VE-821), and ATR (e.g., NU7026).

In some embodiments, the anti-cancer therapy comprises a radiosensitizer. In some embodiments, the methods provided herein comprise administering to the individual a radiosensitizer, e.g., in combination with another anti-cancer therapy. Exemplary radiosensitizers include hypoxia radiosensitizers such as misonidazole, metronidazole, and trans-sodium crocetinate, a compound that helps to increase the diffusion of oxygen into hypoxic tumor tissue. The radiosensitizer can also be a DNA damage response inhibitor interfering with base excision repair (BER), nucleotide excision repair (NER), mismatch repair (MMR), recombinational repair comprising homologous recombination (HR) and non-homologous end-joining (NHEJ), and direct repair mechanisms. Single strand break (SSB) repair mechanisms include BER, NER, or MMR pathways, while double stranded break (DSB) repair mechanisms consist of HR and NHEJ pathways. Radiation causes DNA breaks that, if not repaired, are lethal. SSBs are repaired through a combination of BER, NER and MMR mechanisms using the intact DNA strand as a template. The predominant pathway of SSB repair is BER, utilizing a family of related enzymes termed poly-(ADP-ribose) polymerases (PARP). Thus, the radiosensitizer can include DNA damage response inhibitors such as PARP inhibitors.

In some embodiments, the anti-cancer therapy comprises an anti-inflammatory agent. In some embodiments, the methods provided herein comprise administering to the individual an anti-inflammatory agent, e.g., in combination with another anti-cancer therapy. In some embodiments, the anti-inflammatory agent is an agent that blocks, inhibits, or reduces inflammation or signaling from an inflammatory signaling pathway In some embodiments, the anti-inflammatory agent inhibits or reduces the activity of one or more of any of the following: IL-1, IL-2, IL-3, IL-4, IL-5, IL-6, IL-7, IL-8, IL-9, IL-10, IL-12, IL-13, IL-15, IL-18, IL-23; interferons (IFNs), e.g., IFNα, IFNβ, IFN-γ, IFN-γ inducing factor (IGIF); transforming growth factor-β (TGF-β); transforming growth factor-α (TGF-α); tumor necrosis factors, e.g., TNF-α, TNF-β, TNF-RI, TNF-RII; CD23; CD30; CD40L; EGF; G-CSF; GDNF; PDGF-BB; RANTES/CCL5; IKK; NF-κB; TLR2; TLR3; TLR4; TL5; TLR6; TLR7; TLR8; TLR8; TLR9; and/or any cognate receptors thereof. In some embodiments, the anti-inflammatory agent is an IL-1 or IL-1 receptor antagonist, such as anakinra (Kineret®), rilonacept, or canakinumab. In some embodiments, the anti-inflammatory agent is an IL-6 or IL-6 receptor antagonist, e.g., an anti-IL-6 antibody or an anti-IL-6 receptor antibody, such as tocilizumab (ACTEMRA®), olokizumab, clazakizumab, sarilumab, sirukumab, siltuximab, or ALX-0061. In some embodiments, the anti-inflammatory agent is a TNF-α antagonist, e.g., an anti-TNFα antibody, such as infliximab (Remicade®), golimumab (Simponi®), adalimumab (Humira®), certolizumab pegol (Cimzia®) or etanercept. In some embodiments, the anti-inflammatory agent is a corticosteroid. Exemplary corticosteroids include, but are not limited to, cortisone (hydrocortisone, hydrocortisone sodium phosphate, hydrocortisone sodium succinate, Ala-Cort®, Hydrocort Acetate®, hydrocortone phosphate Lanacort®, Solu-Corteft)), decadron (dexamethasone, dexamethasone acetate, dexamethasone sodium phosphate, Dexasone®, Diodex®, Hexadrol®, Maxidex®), methylprednisolone (6-methylprednisolone, methylprednisolone acetate, methylprednisolone sodium succinate, Duralone®, Medralone®, Medrol®, M-Prednisol®, Solu-Medrol®), prednisolone (Delta-Cortef®, ORAPRED®, Pediapred®, Prezone®), and prednisone (Deltasone®, Liquid Pred®, Meticorten®, Orasone®), and bisphosphonates (e.g., pamidronate (Aredia®), and zoledronic acid (Zometac®).

In some embodiments, the anti-cancer therapy comprises an anti-hormonal agent. In some embodiments, the methods provided herein comprise administering to the individual an anti-hormonal agent, e.g., in combination with another anti-cancer therapy. Anti-hormonal agents are agents that act to regulate or inhibit hormone action on tumors. Examples of anti-hormonal agents include anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGACE® megestrol acetate, AROMASIN® exemestane, formestanie, fadrozole, RIVISOR® vorozole, FEMARA® letrozole, and ARIMIDEX® (anastrozole); anti-androgens such as flutamide, nilutamide, bicalutamide, leuprolide, and goserelin; troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN topoisomerase 1 inhibitor; ABARELIX® rmRH; and pharmaceutically acceptable salts, acids or derivatives of any of the above.

In some embodiments, the anti-cancer therapy comprises an antimetabolite chemotherapeutic agent. In some embodiments, the methods provided herein comprise administering to the individual an antimetabolite chemotherapeutic agent, e.g., in combination with another anti-cancer therapy. Antimetabolite chemotherapeutic agents are agents that are structurally similar to a metabolite, but cannot be used by the body in a productive manner. Many antimetabolite chemotherapeutic agents interfere with the production of RNA or DNA. Examples of antimetabolite chemotherapeutic agents include gemcitabine (GEMZAR®), 5-fluorouracil (5-FU), capecitabine (XELODA™), 6-mercaptopurine, methotrexate, 6-thioguanine, pemetrexed, raltitrexed, arabinosylcytosine ARA-C cytarabine (CYTOSAR-U®), dacarbazine (DTIC-DOMED), azocytosine, deoxycytosine, pyridmidene, fludarabine (FLUDARA), cladrabine, and 2-deoxy-D-glucose. In some embodiments, an antimetabolite chemotherapeutic agent is gemcitabine. Gemcitabine HCl is sold by Eli Lilly under the trademark GEMZAR®.

In some embodiments, the anti-cancer therapy comprises a platinum-based chemotherapeutic agent. In some embodiments, the methods provided herein comprise administering to the individual a platinum-based chemotherapeutic agent, e.g., in combination with another anti-cancer therapy. Platinum-based chemotherapeutic agents are chemotherapeutic agents that comprise an organic compound containing platinum as an integral part of the molecule. In some embodiments, a chemotherapeutic agent is a platinum agent. In some such embodiments, the platinum agent is selected from cisplatin, carboplatin, oxaliplatin, nedaplatin, triplatin tetranitrate, phenanthriplatin, picoplatin, or satraplatin.

In some embodiments, the anti-cancer therapy comprises a heat shock protein (HSP) inhibitor, a MYC inhibitor, an HDAC inhibitor, an immunotherapy, a neoantigen, a vaccine, or a cellular therapy. In some embodiments, the anti-cancer therapy includes one or more of a chemotherapy, a VEGF inhibitor, an Integrin β3 inhibitor, a statin, an EGFR inhibitor, an mTOR inhibitor, a PI3K inhibitor, a MAPK inhibitor, or a CDK4/6 inhibitor.

In some embodiments, the anti-cancer therapy comprises a kinase inhibitor. In some embodiments, the methods provided herein comprise administering to the individual a kinase inhibitor, e.g., in combination with another anti-cancer therapy. In some embodiments, the kinase inhibitor is crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, or TAE684 (NVP-TAE684). Additional examples of ALK kinase inhibitors that may be used according to any of the methods provided herein are described in examples 3-39 of WO2005016894, which is incorporated herein by reference.

In some embodiments, the anti-cancer therapy comprises a heat shock protein (HSP) inhibitor. In some embodiments, the methods provided herein comprise administering to the individual an HSP inhibitor, e.g., in combination with another anti-cancer therapy. In some embodiments, the HSP inhibitor is a Pan-HSP inhibitor, such as KNK423. In some embodiments, the HSP inhibitor is an HSP70 inhibitor, such as cmHsp70.1, quercetin, VER155008, or 17-AAD. In some embodiments, the HSP inhibitor is a HSP90 inhibitor. In some embodiments, the HSP90 inhibitor is 17-AAD, Debio0932, ganetespib (STA-9090), retaspimycin hydrochloride (retaspimycin, IPI-504), AUY922, alvespimycin (KOS-1022, 17-DMAG), tanespimycin (KOS-953, 17-AAG), DS 2248, or AT13387 (onalespib). In some embodiments, the HSP inhibitor is an HSP27 inhibitor, such as Apatorsen (OGX-427).

In some embodiments, the anti-cancer therapy comprises a MYC inhibitor. In some embodiments, the methods provided herein comprise administering to the individual a MYC inhibitor, e.g., in combination with another anti-cancer therapy. In some embodiments, the MYC inhibitor is MYCi361 (NUCC-0196361), MYCi975 (NUCC-0200975), Omomyc (dominant negative peptide), ZINC16293153 (Min9), 10058-F4, JKY-2-169, 7594-0035, or inhibitors of MYC/MAX dimerization and/or MYC/MAX/DNA complex formation.

In some embodiments, the anti-cancer therapy comprises a histone deacetylase (HDAC) inhibitor. In some embodiments, the methods provided herein comprise administering to the individual an HDAC inhibitor, e.g., in combination with another anti-cancer therapy. In some embodiments, the HDAC inhibitor is belinostat (PXD101, Beleodaq®4), SAHA (vorinostat, suberoylanilide hydroxamine, Zolinza®), panobinostat (LBH589, LAQ-824), ACY1215 (Rocilinostat), quisinostat (JNJ-26481585), abexinostat (PCI-24781), pracinostat (SB939), givinostat (ITF2357), resminostat (4SC-201), trichostatin A (TSA), MS-275 (etinostat), Romidepsin (depsipeptide, FK228), MGCDO103 (mocetinostat), BML-210, CAY10603, valproic acid, MC1568, CUDC-907, CI-994 (Tacedinaline), Pivanex (AN-9), AR-42, Chidamide (CS055, HBI-8000), CUDC-101, CHR-3996, MPTOE028, BRD8430, MRLB-223, apicidin, RGFP966, BG45, PCI-34051, C149 (NCC149), TMP269, Cpd2, T247, T326, LMK235, C1A, HPOB, Nexturastat A, Befexamac, CBHA, Phenylbutyrate, MC1568, SNDX275, Scriptaid, Merck60, PX089344, PX105684, PX117735, PX117792, PX117245, PX105844, compound 12 as described by L1 et al., Cold Spring Harb Perspect Med (2016) 6(10):a026831, or PX117445.

In some embodiments, the anti-cancer therapy comprises a VEGF inhibitor. In some embodiments, the methods provided herein comprise administering to the individual a VEGF inhibitor, e.g., in combination with another anti-cancer therapy. In some embodiments, the VEGF inhibitor is Bevacizumab (Avastin®), BMS-690514, ramucirumab, pazopanib, sorafenib, sunitinib, golvatinib, vandetanib, cabozantinib, levantinib, axitinib, cediranib, tivozanib, lucitanib, semaxanib, nindentanib, regorafinib, or aflibercept.

In some embodiments, the anti-cancer therapy comprises an integrin β3 inhibitor. In some embodiments, the methods provided herein comprise administering to the individual an integrin β3 inhibitor, e.g., in combination with another anti-cancer therapy. In some embodiments, the integrin β3 inhibitor is anti-avb3 (clone LM609), cilengitide (EMD121974, NSC, 707544), an siRNA, GLPG0187, MK-0429, CNTO95, TN-161, etaracizumab (MEDI-522), intetumumab (CNTO95) (anti-alphaV subunit antibody), abituzumab (EMD 525797/DI17E6) (anti-alphaV subunit antibody), JSM6427, SJ749, BCH-15046, SCH221153, or SC56631. In some embodiments, the anti-cancer therapy comprises an αIIbβ3 integrin inhibitor. In some embodiments, the methods provided herein comprise administering to the individual an αIIβ3 integrin inhibitor, e.g., in combination with another anti-cancer therapy. In some embodiments, the αIIβ3 integrin inhibitor is abciximab, eptifibatide (Integrilin®), or tirofiban (Aggrastat®).

In some embodiments, the anti-cancer therapy comprises a statin or a statin-based agent. In some embodiments, the methods provided herein comprise administering to the individual a statin or a statin-based agent, e.g., in combination with another anti-cancer therapy. In some embodiments, the statin or statin-based agent is simvastatin, atorvastatin, fluvastatin, pitavastatin, pravastatin, rosuvastatin, or cerivastatin.

In some embodiments, the anti-cancer therapy comprises an mTOR inhibitor. In some embodiments, the methods provided herein comprise administering to the individual an mTOR inhibitor, e.g., in combination with another anti-cancer therapy. In some embodiments, the mTOR inhibitor is temsirolimus (CCI-779), KU-006379, PP242, Torin1, Torin2, ICSN3250, Rapalink-1, CC-223, sirolimus (rapamycin), everolimus (RAD001), dactosilib (NVP-BEZ235), GSK2126458, WAY-001, WAY-600, WYE-687, WYE-354, SF1126, XL765, INK128 (MLN012), AZD8055, OSI027, AZD2014, or AP-23573.

In some embodiments, the anti-cancer therapy comprises a PI3K inhibitor. In some embodiments, the methods provided herein comprise administering to the individual a PI3K inhibitor, e.g., in combination with another anti-cancer therapy. In some embodiments, the PI3K inhibitor is GSK2636771, buparlisib (BKM120), AZD8186, copanlisib (BAY80-6946), LY294002, PX-866, TGX115, TGX126, BEZ235, SF1126, idelalisib (GS-1101, CAL-101), pictilisib (GDC-094), GDC0032, IPI145, INKi 117 (MLNI 117), SAR260301, KIN-193 (AZD6482), duvelisib, GS-9820, GSK2636771, GDC-0980, AMG319, pazobanib, or alpelisib (BYL719, Piqray).

In some embodiments, the anti-cancer therapy comprises a MAPK inhibitor. In some embodiments, the methods provided herein comprise administering to the individual a MAPK inhibitor, e.g., in combination with another anti-cancer therapy. In some embodiments, the MAPK inhibitor is SB203580, SKF-86002, BIRB-796, SC-409, RJW-67657, BIRB-796, VX-745, RO3201195, SB-242235, or MW181.

In some embodiments, the anti-cancer therapy comprises a CDK4/6 inhibitor. In some embodiments, the methods provided herein comprise administering to the individual a CDK4/6 inhibitor, e.g., in combination with another anti-cancer therapy. In some embodiments, the CDK4/6 inhibitor is ribociclib (Kisqali®, LEE011), palbociclib (PD0332991, Ibrance®), or abemaciclib (LY2835219).

In some embodiments, the anti-cancer therapy comprises an EGFR inhibitor. In some embodiments, the methods provided herein comprise administering to the individual an EGFR inhibitor, e.g., in combination with another anti-cancer therapy. In some embodiments, the EGFR inhibitor is cetuximab, panitumumab, lapatinib, gefitinib, vandetanib, dacomitinib, icotinib, osimertinib (AZD9291), afatanib, olmutinib, EGF816 (nazartinib), avitinib (AC0010), rociletinib (CO-1686), BMS-690514, YH5448, PF-06747775, ASP8273, PF299804, AP26113, or erlotinib. In some embodiments, the EGFR inhibitor is gefitinib or cetuximab.

In some embodiments, the anti-cancer therapy comprises a cancer immunotherapy, such as a cancer vaccine, cell-based therapy, T cell receptor (TCR)-based therapy, adjuvant immunotherapy, cytokine immunotherapy, and oncolytic virus therapy. In some embodiments, the methods provided herein comprise administering to the individual a cancer immunotherapy, such as a cancer vaccine, cell-based therapy, T cell receptor (TCR)-based therapy, adjuvant immunotherapy, cytokine immunotherapy, and oncolytic virus therapy, e.g., in combination with another anti-cancer therapy. In some embodiments, the cancer immunotherapy comprises a small molecule, nucleic acid, polypeptide, carbohydrate, toxin, cell-based agent, or cell-binding agent. Examples of cancer immunotherapies are described in greater detail herein but are not intended to be limiting. In some embodiments, the cancer immunotherapy activates one or more aspects of the immune system to attack a cell (e.g., a tumor cell) that expresses a neoantigen, e.g., a neoantigen expressed by a cancer of the disclosure. The cancer immunotherapies of the present disclosure are contemplated for use as monotherapies, or in combination approaches comprising two or more in any combination or number, subject to medical judgement. Any of the cancer immunotherapies (optionally as monotherapies or in combination with another cancer immunotherapy or other therapeutic agent described herein) may find use in any of the methods described herein.

In some embodiments, the cancer immunotherapy comprises a cancer vaccine. A range of cancer vaccines have been tested that employ different approaches to promoting an immune response against a cancer (see, e.g., Emens L A, Expert Opin Emerg Drugs 13(2). 295-308 (2008) and US20190367613). Approaches have been designed to enhance the response of B cells, T cells, or professional antigen-presenting cells against tumors. Exemplary types of cancer vaccines include, but are not limited to, DNA-based vaccines, RNA-based vaccines, virus transduced vaccines, peptide-based vaccines, dendritic cell vaccines, oncolytic viruses, whole tumor cell vaccines, tumor antigen vaccines, etc. In some embodiments, the cancer vaccine can be prophylactic or therapeutic. In some embodiments, the cancer vaccine is formulated as a peptide-based vaccine, a nucleic acid-based vaccine, an antibody based vaccine, or a cell based vaccine. For example, a vaccine composition can include naked cDNA in cationic lipid formulations; lipopeptides (e.g., Vitiello, A. et ah, J. Clin. Invest. 95:341, 1995), naked cDNA or peptides, encapsulated e.g., in poly(DL-lactide-co-glycolide) (“PLG”) microspheres (see, e.g., Eldridge, et ah, Molec. Immunol. 28:287-294, 1991: Alonso et al, Vaccine 12:299-306, 1994; Jones et al, Vaccine 13:675-681, 1995); peptide composition contained in immune stimulating complexes (iSCOMS) (e.g., Takahashi et al, Nature 344:873-875, 1990; Hu et al, Clin. Exp. Immunol. 113:235-243, 1998); or multiple antigen peptide systems (MAPs) (see e.g., Tam, J. P., Proc. Natd Acad. Sci. U.S.A. 85:5409-5413, 1988; Tam, J. P., J. Immunol. Methods 196: 17-32, 1996). In some embodiments, a cancer vaccine is formulated as a peptide-based vaccine, or nucleic acid based vaccine in which the nucleic acid encodes the polypeptides. In some embodiments, a cancer vaccine is formulated as an antibody-based vaccine. In some embodiments, a cancer vaccine is formulated as a cell based vaccine. In some embodiments, the cancer vaccine is a peptide cancer vaccine, which in some embodiments is a personalized peptide vaccine. In some embodiments, the cancer vaccine is a multivalent long peptide, a multiple peptide, a peptide mixture, a hybrid peptide, or a peptide pulsed dendritic cell vaccine (see, e.g., Yamada et al, Cancer Sci, 104: 14-21), 2013). In some embodiments, such cancer vaccines augment the anti-cancer response.

In some embodiments, the cancer vaccine comprises a polynucleotide that encodes a neoantigen, e.g., a neoantigen expressed by a cancer of the disclosure. In some embodiments, the cancer vaccine comprises DNA or RNA that encodes a neoantigen. In some embodiments, the cancer vaccine comprises a polynucleotide that encodes a neoantigen. In some embodiments, the cancer vaccine further comprises one or more additional antigens, neoantigens, or other sequences that promote antigen presentation and/or an immune response. In some embodiments, the polynucleotide is complexed with one or more additional agents, such as a liposome or lipoplex. In some embodiments, the polynucleotide(s) are taken up and translated by antigen presenting cells (APCs), which then present the neoantigen(s) via MHC class I on the APC cell surface.

In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, the cancer vaccine is selected from an oncolytic viral therapy such as pexastimogene devacirepvec (PexaVec/JX-594, SillaJen/formerly Jennerex Biotherapeutics), a thymidine kinase-(TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543). prostate cancer (NCT01619813), head and neck squamous cell cancer (NCT01166542), pancreatic adenocarcinoma (NCT00998322), and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117), metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676), and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260), fallopian tube cancer, ovarian cancer (NCT 02759588); or CG0070 (Cold Genesys), an adenovirus engineered to express GM-CSF in bladder cancer (NCT02365818); anti-gp100; STINGVAX; GVAX; DCVaxL; and DNX-2401. In some embodiments, the cancer vaccine is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TGO1 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8⁺ T cell response. In some embodiments, the cancer vaccine comprises a vector-based tumor antigen vaccine. Vector-based tumor antigen vaccines can be used as a way to provide a steady supply of antigens to stimulate an anti-tumor immune response. In some embodiments, vectors encoding for tumor antigens are injected into an individual (possibly with pro-inflammatory or other attractants such as GM-CSF), taken up by cells in vivo to make the specific antigens, which then provoke the desired immune response. In some embodiments, vectors may be used to deliver more than one tumor antigen at a time, to increase the immune response. In addition, recombinant virus, bacteria or yeast vectors can trigger their own immune responses, which may also enhance the overall immune response.

In some embodiments, the cancer vaccine comprises a DNA-based vaccine. In some embodiments, DNA-based vaccines can be employed to stimulate an anti-tumor response. The ability of directly injected DNA that encodes an antigenic protein, to elicit a protective immune response has been demonstrated in numerous experimental systems. Vaccination through directly injecting DNA that encodes an antigenic protein, to elicit a protective immune response often produces both cell-mediated and humoral responses. Moreover, reproducible immune responses to DNA encoding various antigens have been reported in mice that last essentially for the lifetime of the animal (see, e.g., Yankauckas et al. (1993) DNA Cell Biol., 12: 771-776). In some embodiments, plasmid (or other vector) DNA that includes a sequence encoding a protein operably linked to regulatory elements required for gene expression is administered to individuals (e.g. human patients, non-human mammals, etc.). In some embodiments, the cells of the individual take up the administered DNA and the coding sequence is expressed. In some embodiments, the antigen so produced becomes a target against which an immune response is directed.

In some embodiments, the cancer vaccine comprises an RNA-based vaccine. In some embodiments, RNA-based vaccines can be employed to stimulate an anti-tumor response. In some embodiments, RNA-based vaccines comprise a self-replicating RNA molecule. In some embodiments, the self-replicating RNA molecule may be an alphavirus-derived RNA replicon. Self-replicating RNA (or “SAM”) molecules are well known in the art and can be produced by using replication elements derived from, e.g., alphaviruses, and substituting the structural viral proteins with a nucleotide sequence encoding a protein of interest. A self-replicating RNA molecule is typically a +-strand molecule which can be directly translated after delivery to a cell, and this translation provides a RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded polypeptide, or may be transcribed to provide further transcripts with the same sense as the delivered RNA which are translated to provide in situ expression of the antigen.

In some embodiments, the cancer immunotherapy comprises a cell-based therapy. In some embodiments, the cancer immunotherapy comprises a T cell-based therapy. In some embodiments, the cancer immunotherapy comprises an adoptive therapy, e.g., an adoptive T cell-based therapy. In some embodiments, the T cells are autologous or allogeneic to the recipient. In some embodiments, the T cells are CD8+ T cells. In some embodiments, the T cells are CD4+ T cells. Adoptive immunotherapy refers to a therapeutic approach for treating cancer or infectious diseases in which immune cells are administered to a host with the aim that the cells mediate either directly or indirectly specific immunity to (i.e., mount an immune response directed against) cancer cells. In some embodiments, the immune response results in inhibition of tumor and/or metastatic cell growth and/or proliferation, and in related embodiments, results in neoplastic cell death and/or resorption. The immune cells can be derived from a different organism/host (exogenous immune cells) or can be cells obtained from the subject organism (autologous immune cells). In some embodiments, the immune cells (e.g., autologous or allogeneic T cells (e.g., regulatory T cells, CD4+ T cells, CD8+ T cells, or gamma-delta T cells), NK cells, invariant NK cells, or NKT cells) can be genetically engineered to express antigen receptors such as engineered TCRs and/or chimeric antigen receptors (CARs). For example, the host cells (e.g., autologous or allogeneic T-cells) are modified to express a T cell receptor (TCR) having antigenic specificity for a cancer antigen. In some embodiments, NK cells are engineered to express a TCR. The NK cells may be further engineered to express a CAR. Multiple CARs and/or TCRs, such as to different antigens, may be added to a single cell type, such as T cells or NK cells. In some embodiments, the cells comprise one or more nucleic acids/expression constructs/vectors introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g. chimeric). In some embodiments, a population of immune cells can be obtained from a subject in need of therapy or suffering from a disease associated with reduced immune cell activity. Thus, the cells will be autologous to the subject in need of therapy. In some embodiments, a population of immune cells can be obtained from a donor, such as a histocompatibility-matched donor. In some embodiments, the immune cell population can be harvested from the peripheral blood, cord blood, bone marrow, spleen, or any other organ/tissue in which immune cells reside in said subject or donor. In some embodiments, the immune cells can be isolated from a pool of subjects and/or donors, such as from pooled cord blood. In some embodiments, when the population of immune cells is obtained from a donor distinct from the subject, the donor may be allogeneic, provided the cells obtained are subject-compatible, in that they can be introduced into the subject. In some embodiments, allogeneic donor cells may or may not be human-leukocyte-antigen (HLA)-compatible. In some embodiments, to be rendered subject-compatible, allogeneic cells can be treated to reduce immunogenicity.

In some embodiments, the cell-based therapy comprises a T cell-based therapy, such as autologous cells, e.g., tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. Several approaches for the isolation, derivation, engineering or modification, activation, and expansion of functional anti-tumor effector cells have been described in the last two decades and may be used according to any of the methods provided herein. In some embodiments, the T cells are derived from the blood, bone marrow, lymph, umbilical cord, or lymphoid organs. In some embodiments, the cells are human cells. In some embodiments, the cells are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, the cells may be allogeneic and/or autologous. In some embodiments, such as for off-the-shelf technologies, the cells are pluripotent and/or multipotent, such as stem cells, such as induced pluripotent stem cells (iPSCs).

In some embodiments, the T cell-based therapy comprises a chimeric antigen receptor (CAR)-T cell-based therapy. This approach involves engineering a CAR that specifically binds to an antigen of interest and comprises one or more intracellular signaling domains for T cell activation. The CAR is then expressed on the surface of engineered T cells (CAR-T) and administered to a patient, leading to a T-cell-specific immune response against cancer cells expressing the antigen.

In some embodiments, the T cell-based therapy comprises T cells expressing a recombinant T cell receptor (TCR). This approach involves identifying a TCR that specifically binds to an antigen of interest, which is then used to replace the endogenous or native TCR on the surface of engineered T cells that are administered to a patient, leading to a T-cell-specific immune response against cancer cells expressing the antigen.

In some embodiments, the T cell-based therapy comprises tumor-infiltrating lymphocytes (TILs). For example, TILs can be isolated from a tumor or cancer of the present disclosure, then isolated and expanded in vitro. Some or all of these TILs may specifically recognize an antigen expressed by the tumor or cancer of the present disclosure. In some embodiments, the TILs are exposed to one or more neoantigens, e.g., a neoantigen, in vitro after isolation. TILs are then administered to the patient (optionally in combination with one or more cytokines or other immune-stimulating substances).

In some embodiments, the cell-based therapy comprises a natural killer (NK) cell-based therapy. Natural killer (NK) cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells can be detected by specific surface markers, such as CD16, CD56, and CD8 in humans. NK cells do not express T-cell antigen receptors, the pan T marker CD3, or surface immunoglobulin B cell receptors. In some embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art.

In some embodiments, the cell-based therapy comprises a dendritic cell (DC)-based therapy, e.g., a dendritic cell vaccine. In some embodiments, the DC vaccine comprises antigen-presenting cells that are able to induce specific T cell immunity, which are harvested from the patient or from a donor. In some embodiments, the DC vaccine can then be exposed in vitro to a peptide antigen, for which T cells are to be generated in the patient. In some embodiments, dendritic cells loaded with the antigen are then injected back into the patient. In some embodiments, immunization may be repeated multiple times if desired. Methods for harvesting, expanding, and administering dendritic cells are known in the art, see, e.g., WO2019178081. Dendritic cell vaccines (such as Sipuleucel-T, also known as APC8015 and PROVENGE®) are vaccines that involve administration of dendritic cells that act as APCs to present one or more cancer-specific antigens to the patient's immune system. In some embodiments, the dendritic cells are autologous or allogeneic to the recipient.

In some embodiments, the cancer immunotherapy comprises a TCR-based therapy. In some embodiments, the cancer immunotherapy comprises administration of one or more TCRs or TCR-based therapeutics that specifically bind an antigen expressed by a cancer of the present disclosure. In some embodiments, the TCR-based therapeutic may further include a moiety that binds an immune cell (e.g., a T cell), such as an antibody or antibody fragment that specifically binds a T cell surface protein or receptor (e.g., an anti-CD3 antibody or antibody fragment).

In some embodiments, the immunotherapy comprises adjuvant immunotherapy. Adjuvant immunotherapy comprises the use of one or more agents that activate components of the innate immune system, e.g., HILTONOL® (imiquimod), which targets the TLR7 pathway.

In some embodiments, the immunotherapy comprises cytokine immunotherapy. Cytokine immunotherapy comprises the use of one or more cytokines that activate components of the immune system. Examples include, but are not limited to, aldesleukin (PROLEUKIN®; interleukin-2), interferon alfa-2a (ROFERON®-A), interferon alfa-2b (INTRON®-A), and peginterferon alfa-2b (PEGINTRON®).

In some embodiments, the immunotherapy comprises oncolytic virus therapy. Oncolytic virus therapy uses genetically modified viruses to replicate in and kill cancer cells, leading to the release of antigens that stimulate an immune response. In some embodiments, replication-competent oncolytic viruses expressing a tumor antigen comprise any naturally occurring (e.g., from a “field source”) or modified replication-competent oncolytic virus. In some embodiments, the oncolytic virus, in addition to expressing a tumor antigen, may be modified to increase selectivity of the virus for cancer cells. In some embodiments, replication-competent oncolytic viruses include, but are not limited to, oncolytic viruses that are a member in the family of myoviridae, siphoviridae, podpviridae, teciviridae, corticoviridae, plasmaviridae, lipothrixviridae, fuselloviridae, poxyiridae, iridoviridae, phycodnaviridae, baculoviridae, herpesviridae, adnoviridae, papovaviridae, polydnaviridae, inoviridae, microviridae, geminiviridae, circoviridae, parvoviridae, hcpadnaviridae, retroviridae, cyctoviridae, reoviridae, birnaviridae, paramyxoviridae, rhabdoviridae, filoviridae, orthomyxoviridae, bunyaviridae, arenaviridae, Leviviridae, picornaviridae, sequiviridae, comoviridae, potyviridae, caliciviridae, astroviridae, nodaviridae, tetraviridae, tombusviridae, coronaviridae, glaviviridae, togaviridae, and barnaviridae. In some embodiments, replication-competent oncolytic viruses include adenovirus, retrovirus, reovirus, rhabdovirus, Newcastle Disease virus (NDV), polyoma virus, vaccinia virus (VacV), herpes simplex virus, picornavirus, coxsackie virus and parvovirus. In some embodiments, a replicative oncolytic vaccinia virus expressing a tumor antigen may be engineered to lack one or more functional genes in order to increase the cancer selectivity of the virus. In some embodiments, an oncolytic vaccinia virus is engineered to lack thymidine kinase (TK) activity. In some embodiments, the oncolytic vaccinia virus may be engineered to lack vaccinia virus growth factor (VGF). In some embodiments, an oncolytic vaccinia virus may be engineered to lack both VGF and TK activity. In some embodiments, an oncolytic vaccinia virus may be engineered to lack one or more genes involved in evading host interferon (IFN) response such as E3L, K3L, B18R, or B8R In some embodiments, a replicative oncolytic vaccinia virus is a Western Reserve, Copenhagen, Lister or Wyeth strain and lacks a functional TK gene. In some embodiments, the oncolytic vaccinia virus is a Western Reserve, Copenhagen, Lister or Wyeth strain lacking a functional B18R and/or B8R gene. In some embodiments, a replicative oncolytic vaccinia virus expressing a tumor antigen may be locally or systemically administered to a subject, e.g. via intratumoral, intraperitoneal, intravenous, intra-arterial, intramuscular, intradermal, intracranial, subcutaneous, or intranasal administration.

The following exemplary embodiments are representative of some aspects of the invention:

Embodiment 1. A method of detecting loss-of-heterozygosity (LOH) of a human leukocyte antigen (HLA) gene, comprising:

providing a plurality of nucleic acids obtained from a sample from an individual, wherein the plurality of nucleic acids comprises nucleic acids encoding an HLA gene;

optionally, ligating one or more adaptors onto one or more nucleic acids from the plurality of nucleic acids;

amplifying nucleic acids from the plurality of nucleic acids;

capturing a plurality of nucleic acids corresponding to the HLA gene, wherein the plurality of nucleic acids corresponding to the HLA gene is captured from the amplified nucleic acids by hybridization with a bait molecule;

sequencing, by a sequencer, the captured nucleic acids to obtain a plurality of sequence reads corresponding to the HLA gene;

fitting, by one or more processors, one or more values associated with one or more of the plurality of sequence reads to a model; and

based on the model, detecting LOH of the HLA gene and a relative binding propensity for an HLA allele of the HLA gene.

Embodiment 2. The method of embodiment 1, wherein LOH of the HLA gene and relative binding propensity for an HLA allele of the HLA gene are detected by:
a) obtaining an observed allele frequency for an HLA allele, wherein observed allele frequency corresponds to frequency of nucleic acid(s) encoding at least a portion of the HLA allele as detected among the plurality of sequence reads corresponding to the HLA gene;
b) obtaining a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles;
c) applying an objective function to measure a difference between the relative binding propensity and the observed allele frequency of the HLA allele;
d) applying an optimization model to minimize the objective function;
e) determining an adjusted allele frequency of the HLA allele based on the optimization model and the observed allele frequency; and
f) determining that LOH has occurred when the adjusted allele frequency of the HLA allele is less than a predetermined threshold.
   Embodiment 3. The method of embodiment 1 or embodiment 2, further comprising, based at least in part on detection of LOH of the HLA gene, administering an effective amount of a treatment other than an immune checkpoint inhibitor (ICI) to the individual.
   Embodiment 4. The method of embodiment 1 or embodiment 2, further comprising, based at least in part on detection of LOH of the HLA gene, recommending a treatment other than an immune checkpoint inhibitor (ICI).
   Embodiment 5. The method of embodiment 1 or embodiment 2, further comprising: detecting, or acquiring knowledge of, a high tumor mutational burden (TMB) in the sample.
   Embodiment 6. The method of embodiment 5, further comprising, based at least in part on detection of LOH of the HLA gene and high TMB, administering an effective amount of an immune checkpoint inhibitor (ICI) to the individual.
   Embodiment 7. The method of embodiment 5, further comprising, based at least in part on detection of LOH of the HLA gene and high TMB, recommending a treatment comprising an immune checkpoint inhibitor (ICI) to the individual.
   Embodiment 8. The method of any one of embodiments 1-7, wherein the HLA gene is a human HLA-A, HLA-B, or HLA-C gene.
   Embodiment 9. The method of any one or embodiments 1-8, further comprising, prior to (1), extracting the plurality of nucleic acids from the sample.
   Embodiment 10. The method of any one of embodiments 1-9, wherein the sample comprises tumor cells and/or tumor nucleic acids.
   Embodiment 11. The method of embodiment 10, wherein the sample further comprises non-tumor cells.
   Embodiment 12. The method of embodiment 10, wherein the sample is from a tumor biopsy or tumor specimen.
   Embodiment 13. The method of embodiment 10, wherein the sample comprises tumor cell-free DNA (cfDNA).
   Embodiment 14. The method of embodiment 10, wherein the sample comprises fluid, cells, or tissue.
   Embodiment 15. The method of embodiment 14, wherein the sample comprises blood or plasma.
   Embodiment 16. The method of embodiment 10, wherein the sample comprises a tumor biopsy or a circulating tumor cell.
   Embodiment 17. The method of embodiment 16, wherein the sample from the individual is a nucleic acid sample.
   Embodiment 18. The method of embodiment 17, wherein the nucleic acid sample comprises mRNA, genomic DNA, circulating tumor DNA, cell-free DNA, or cell-free RNA. Embodiment 19. The method of any one of embodiments 5-18, wherein the TMB is determined based on a number of non-driver somatic coding mutations per megabase of genome sequenced.
   Embodiment 20. A method comprising:

identifying a plurality of chemical reactions such that:
each reaction corresponds to a bait molecule binding to a different allele of a polymorphic gene, and each reaction resulting in capture of a corresponding allele fraction;
the plurality of chemical reactions consists of a first subset of reactions and a second subset of reactions, in which the first and second subsets share no reaction in common and in which the first and second subsets each comprise at least one chemical reaction;

identifying a plurality of equations that collectively relate binding propensities of each chemical reaction and allele fraction of each captured allele;

empirically identifying the relative binding propensities of the first subset of the plurality of chemical reactions; and

identifying the relative binding propensities of the second subset by minimizing a total error.

Embodiment 21. The method of embodiment 20, wherein minimizing the total error is subject to a constraint that the median relative binding propensities is equal to 1.
Embodiment 22. The method of embodiment 20, wherein one relative binding propensity is set equal to 1.
Embodiment 23. The method of embodiment 20, wherein minimizing the total error includes performing a least squares procedure.
Embodiment 24. The method of embodiment 20, further comprising:

performing a hybrid capture process to measure raw allele frequencies in a DNA sample of a patient; and

using the first and second subsets of relative binding propensities to scale the measured raw allele frequencies, thereby mitigating sampling bias.

Embodiment 25. The method of embodiment 20, wherein the polymorphic gene includes a Human Leukocyte Antigen gene.
Embodiment 26. The method of embodiment 20, wherein the polymorphic gene is ST7/RAY1, ARH1/NOEY2, TSLC1, RB, PTEN, SMAD2, SMAD4, DCC, TP53, ATM, miR-15a, miR-16-1, NAT2, BRCA1, BRCA2, hOGG1, CDH1, IGF2, CDKN1C/P57, MEN1, PRKAR1A, H19, KRAS, BAP1, PTCH1, SMO, SUFU, NOTCH1, PPP6C, LATS1, CASP8, PTPN14, ARID1A, FBXW7, M6P/IGF2R, IFN-alpha, an olfactory receptor gene, CBFA2T3, DUTT1, FHIT, APC, P16, FCMD, TSC2, miR-34, c-MPL, RUNX3, DIRAS3, NRAS, miR-9, FAM50B, PLAGL1, ER, FLT3, ZDBF2, GPR1, c-KIT, NAP1L5, GRB10, EGFR, PEG10, BRAF, MEST, JAK2, DAPK1, LIT1, WT1, NF-1, PR, c-CBL, DLK1, AKT1, SNURF, a cytochrome P450 gene (CYP), ZNF587, SOCS1, TIMP2, RUNX1, AR, CEBPA, C19MC, EMP3, ZNF331, CDKN2A, PEG3, NNAT, GNAS, or GATA5.
Embodiment 27. The method of embodiment 24, further comprising determining whether the patient has experienced a loss of heterozygosity.
Embodiment 28. A system, comprising:

one or more processors; and

a memory configured to store one or more computer program instructions, wherein the one or more computer program instructions when executed by the one or more processors are configured to:
identify a plurality of chemical reactions such that:
   each reaction corresponds to a bait molecule binding to a different allele of a polymorphic gene, and each reaction resulting in capture of a corresponding allele fraction;
   the plurality of chemical reactions consists of a first subset of reactions and a second subset of reactions, in which the first and second subsets share no reaction in common and in which the first and second subsets each comprise at least one chemical reaction;
identify a plurality of equations that collectively relate binding propensities of each chemical reaction and allele fraction of each captured allele;
receive empirically identified relative binding propensities of the first subset of the plurality of chemical reactions; and
identify the relative binding propensities of the second subset by minimizing a total error.
   Embodiment 29. The system of embodiment 28, wherein minimizing the total error is subject to a constraint that a median relative binding propensity is equal to 1.
   Embodiment 30. The system of embodiment 28, wherein one relative binding propensity is set equal to 1.
   Embodiment 31. The system of embodiment 28, wherein minimizing the total error includes performing a least squares procedure.
   Embodiment 32. The system of embodiment 28, wherein the one or more computer program instructions when executed by the one or more processors are further configured to:

receiving, at the one or more processors, measured raw allele frequencies in a DNA sample of a patient, wherein the measured raw allele frequencies were measured by performing a hybrid capture process; and

scaling, at the one or more processors, the measured raw allele frequencies using the first and second subsets of relative binding propensities, thereby mitigating sampling bias.

Embodiment 33. The system of embodiment 28, wherein the polymorphic gene includes a Human Leukocyte Antigen gene.
Embodiment 34. The system of embodiment 28, wherein the polymorphic gene is ST7/RAY1, ARH1/NOEY2, TSLC1, RB, PTEN, SMAD2, SMAD4, DCC, TP53, ATM, miR-15a, miR-16-1, NAT2, BRCA1, BRCA2, hOGG1, CDH1, IGF2, CDKN1C/P57, MEN1, PRKAR1A, H19, KRAS, BAP1, PTCH1, SMO, SUFU, NOTCH1, PPP6C, LATS1, CASP8, PTPN14, ARID1A, FBXW7, M6P/IGF2R, IFN-alpha, an olfactory receptor gene, CBFA2T3, DUTT1, FHIT, APC, P16, FCMD, TSC2, miR-34, c-MPL, RUNX3, DIRAS3, NRAS, miR-9, FAM50B, PLAGL1, ER, FLT3, ZDBF2, GPR1, c-KIT, NAP1L5, GRB10, EGFR, PEG10, BRAF, MEST, JAK2, DAPK1, LIT1, WT1, NF-1, PR, c-CBL, DLK1, AKT1, SNURF, a cytochrome P450 gene (CYP), ZNF587, SOCS1, TIMP2, RUNX1, AR, CEBPA, C19MC, EMP3, ZNF331, CDKN2A, PEG3, NNAT, GNAS, or GATA5.
Embodiment 35. The system of embodiment 32, wherein the method further comprises determining, at the one or more processors, whether the patient has experienced a loss of heterozygosity.
Embodiment 36. A method for determining allele frequency, comprising:
a) receiving, at one or more processors, an observed allele frequency for an allele of a gene, wherein the observed allele frequency corresponds to frequency of nucleic acid(s) encoding at least a portion of the allele as detected among a plurality of sequence reads corresponding to the gene, wherein the plurality of sequence reads was obtained by sequencing nucleic acids encoding the gene or a portion thereof as captured by hybridization with a bait molecule;
b) receiving, at one or more processors, a relative binding propensity for the allele to the bait molecule, wherein the relative binding propensity of the allele corresponds to propensity of nucleic acid encoding at least a portion of the allele to bind the bait molecule in the presence of nucleic acids encoding portions of one or more other alleles of the gene;
c) executing, by the one or more processors, an objective function to measure a difference between the relative binding propensity and the observed allele frequency of the allele;
d) executing, by the one or more processors, an optimization model to minimize the objective function; and
e) determining, by the one or more processors, an adjusted allele frequency of the allele based on the optimization model and the observed allele frequency.
   Embodiment 37. The method of embodiment 36, wherein the optimization model is a least squares optimization model.
   Embodiment 38. The method of embodiment 36 or embodiment 37, wherein the optimization model is subject to one or more constraints.
   Embodiment 39. The method of embodiment 38, wherein the one or more constraints require that a median value of the relative binding propensities for a plurality of alleles of the gene is equal to 1.
   Embodiment 40. The method of any one of embodiments 36-39, wherein the observed allele frequency corresponds to relative frequency of nucleic acid(s) encoding at least a portion of the allele as detected among the plurality of sequence reads, as compared to a reference value.
   Embodiment 41. The method of embodiment 40, wherein the reference value is a total number of sequence reads.
   Embodiment 42. The method of embodiment 40, wherein the reference value is a number of sequence reads corresponding to a reference gene.
   Embodiment 43. The method of any one of embodiments 36-42, wherein the gene is a human leukocyte antigen (HLA) gene encoding a major histocompatibility (MHC) class I molecule.
   Embodiment 44. The method of any one of embodiments 36-42, wherein the gene is ST7/RAY1, ARH1/NOEY2, TSLC1, RB, PTEN, SMAD2, SMAD4, DCC, TP53, ATM, miR-15a, miR-16-1, NAT2, BRCA1, BRCA2, hOGG1, CDH1, IGF2, CDKN1C/P57, MEN1, PRKAR1A, H19, KRAS, BAP1, PTCH1, SMO, SUFU, NOTCH1, PPP6C, LATS1, CASP8, PTPN14, ARID1A, FBXW7, M6P/IGF2R, IFN-alpha, an olfactory receptor gene, CBFA2T3, DUTT1, FHIT, APC, P16, FCMD, TSC2, miR-34, c-MPL, RUNX3, DIRAS3, NRAS, miR-9, FAM50B, PLAGL1, ER, FLT3, ZDBF2, GPR1, c-KIT, NAP1L5, GRB10, EGFR, PEG10, BRAF, MEST, JAK2, DAPK1, LIT1, WT1, NF-1, PR, c-CBL, DLK1, AKT1, SNURF, a cytochrome P450 gene (CYP), ZNF587, SOCS1, TIMP2, RUNX1, AR, CEBPA, C19MC, EMP3, ZNF331, CDKN2A, PEG3, NNAT, GNAS, or GATA5.
   Embodiment 45. The method of any one of embodiments 36-44, further comprising, after determining the adjusted allele frequency: determining that the gene has undergone loss-of-heterozygosity (LOH) based at least in part on the adjusted allele frequency.
   Embodiment 46. The method of any one of embodiments 36-45, wherein the plurality of sequence reads was obtained by performing next-generation sequencing (NGS), whole exome sequencing, or methylation sequencing on nucleic acids captured by hybridization with the bait molecule.
   Embodiment 47. The method of any one of embodiments 36-46, further comprising, prior to receiving the observed allele frequency: sequencing a plurality of polynucleotides by next-generation sequencing (NGS), whole exome sequencing, or methylation sequencing in order to obtain the plurality of sequence reads, wherein the plurality of polynucleotides comprises nucleic acid(s) encoding at least a portion of the allele.
   Embodiment 48. The method of embodiment 47, further comprising, prior to sequencing the plurality of polynucleotides:

contacting a mixture of polynucleotides with the bait molecule under conditions suitable for hybridization, wherein the mixture comprises a plurality of polynucleotides capable of hybridization with the bait molecule; and

isolating a plurality of polynucleotides that hybridized with the bait molecule, wherein the isolated plurality of polynucleotides that hybridized with the bait molecule are sequenced.

Embodiment 49. The method of embodiment 48, further comprising, prior to contacting the mixture of polynucleotides with the bait molecule:

obtaining a sample from an individual, wherein the sample comprises tumor cells and/or tumor nucleic acids; and

extracting the mixture of polynucleotides from the sample, wherein the mixture of polynucleotides is from the tumor cells and/or tumor nucleic acids.

Embodiment 50. The method of embodiment 49, wherein the sample further comprises non-tumor cells.
Embodiment 51. The method of embodiment 49, wherein the sample is from a tumor biopsy or tumor specimen.
Embodiment 52. The method of embodiment 49, wherein the sample comprises tumor cell-free DNA (cfDNA).
Embodiment 53. The method of any one of embodiments 36-52, further comprising:

(1) receiving, at one or more processors, an observed allele frequency for each of two or more alleles of a gene, wherein the observed allele frequencies correspond to frequency of nucleic acid(s) encoding at least a portion of the respective allele as detected among a plurality of sequence reads corresponding to the gene, wherein the plurality of sequence reads was obtained by sequencing nucleic acids encoding the gene or a portion thereof as captured by hybridization with a bait molecule;

(2) receiving, at one or more processors, a relative binding propensity for each of two or more alleles to the bait molecule, wherein a second of the two or more alleles has a lower relative binding propensity to the bait molecule than a first of the two or more alleles; and

(3) identifying, by the one or more processors, a second bait molecule, wherein the second of the two or more alleles has a higher relative binding propensity to the second bait molecule than to the first bait molecule.

Embodiment 54. The method of embodiment 53, wherein the second bait molecule comprises a sequence complementary to at least a portion of the second of the two or more alleles.
Embodiment 55. A non-transitory computer-readable storage medium comprising one or more programs for execution by one or more processors of a device, the one or more programs including instructions which, when executed by the one or more processors, cause the device to perform the method of any one of embodiments 36-46, 53, and 54.
Embodiment 56. A method for detecting loss-of-heterozygosity (LOH) of a human leukocyte antigen (HLA) gene, comprising:
a) receiving, at one or more processors, an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule;
b) receiving, at one or more processors, a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles;
c) executing, by the one or more processors, an objective function to measure a difference between the relative binding propensity and the observed allele frequency of the HLA allele;
d) executing, by the one or more processors, an optimization model to minimize the objective function;
e) determining, by the one or more processors, an adjusted allele frequency of the HLA allele based on the optimization model and the observed allele frequency; and
f) determining, by the one or more processors, that LOH has occurred when the adjusted allele frequency of the HLA allele is less than a predetermined threshold.
   Embodiment 57. The method of embodiment 56, wherein the HLA gene is a human HLA-A, HLA-B, or HLA-C gene.
   Embodiment 58. The method of embodiment 56 or embodiment 57, wherein the plurality of sequence reads was obtained by sequencing nucleic acids obtained from a sample comprising tumor cells and/or tumor nucleic acids.
   Embodiment 59. The method of embodiment 58, wherein the sample further comprises non-tumor cells.
   Embodiment 60. The method of embodiment 58, wherein the sample is from a tumor biopsy or tumor specimen.
   Embodiment 61. The method of embodiment 58, wherein the sample comprises tumor cell-free DNA (cfDNA).
   Embodiment 62. The method of embodiment 58, wherein the sample comprises fluid, cells, or tissue.
   Embodiment 63. The method of embodiment 62, wherein the sample comprises blood or plasma.
   Embodiment 64. The method of embodiment 58, wherein the sample comprises a tumor biopsy or a circulating tumor cell.
   Embodiment 65. The method of embodiment 58, wherein the sample is a nucleic acid sample.
   Embodiment 66. The method of embodiment 65, wherein the nucleic acid sample comprises mRNA, genomic DNA, circulating tumor DNA, cell-free DNA, or cell-free RNA.
   Embodiment 67. A method of identifying an individual having cancer who may benefit from a treatment comprising an immune checkpoint inhibitor (ICI), the method comprising detecting loss-of-heterozygosity (LOH) of a human leukocyte antigen (HLA) gene in a sample from the individual, wherein LOH of the HLA gene is detected according to the method of any one of embodiments 36-66.
   Embodiment 68. A method of selecting a therapy for an individual having cancer, the method comprising detecting loss-of-heterozygosity (LOH) of a human leukocyte antigen (HLA) gene in a sample from the individual, wherein LOH of the HLA gene is detected according to the method of any one of embodiments 36-66.
   Embodiment 69. A method of identifying one or more treatment options for an individual having cancer, the method comprising:

(a) acquiring knowledge of loss-of-heterozygosity (LOH) of a human leukocyte antigen (HLA) gene in a sample from the individual, wherein LOH of the HLA gene is detected according to the method of any one of embodiments 36-66; and

(b) generating a report comprising one or more treatment options identified for the individual based at least in part on said knowledge.

Embodiment 70. The method of any one of embodiments 67-69, wherein LOH of the HLA gene in the sample indicates that the individual is not likely to benefit from a treatment comprising an ICI.
Embodiment 71. The method of embodiment 70, wherein the one or more treatment options do not include treatment comprising an ICI.
Embodiment 72. A method of selecting treatment for an individual having cancer, comprising acquiring knowledge of loss-of-heterozygosity (LOH) of a human leukocyte antigen (HLA) gene in a sample from an individual having cancer, wherein LOH of the HLA gene is detected according to the method of any one of embodiments 36-66, and wherein responsive to the acquisition of said knowledge: (i) the individual is classified as a candidate not to receive treatment with an immune checkpoint inhibitor (ICI); (ii) the individual is identified as not likely to respond to a treatment that comprises an immune checkpoint inhibitor (ICI); and/or (iii) the individual is classified as a candidate to receive a treatment other than an immune checkpoint inhibitor (ICI).
Embodiment 73. A method of predicting survival of an individual having cancer treated with an immune checkpoint inhibitor (ICI), comprising acquiring knowledge of loss-of-heterozygosity (LOH) of a human leukocyte antigen (HLA) gene in a sample from the individual, wherein LOH of the HLA gene is detected according to the method of any one of embodiments 36-66, and wherein responsive to the acquisition of said knowledge, the individual is predicted to have shorter survival after treatment with the ICI, as compared to survival of an individual treated with the ICI whose cancer does not exhibit LOH of the HLA gene.
Embodiment 74. A method of monitoring an individual having cancer, comprising acquiring knowledge of loss-of-heterozygosity (LOH) of a human leukocyte antigen (HLA) gene in a sample from the individual, wherein LOH of the HLA gene is detected according to the method of any one of embodiments 36-66, and wherein responsive to the acquisition of said knowledge, the individual is predicted to have increased risk of recurrence, as compared to an individual whose cancer does not exhibit LOH of the HLA gene.
Embodiment 75. A method of evaluating an individual having cancer, comprising acquiring knowledge of loss-of-heterozygosity (LOH) of a human leukocyte antigen (HLA) gene in a sample from the individual, wherein LOH of the HLA gene is detected according to the method of any one of embodiments 36-66, and wherein the LOH of the HLA gene identifies the individual as having increased risk of recurrence, as compared to an individual whose cancer does not exhibit LOH of the HLA gene.
Embodiment 76. A method of screening an individual having cancer, comprising acquiring knowledge of loss-of-heterozygosity (LOH) of a human leukocyte antigen (HLA) gene in a sample from the individual, wherein LOH of the HLA gene is detected according to the method of any one of embodiments 36-66, and wherein responsive to the acquisition of said knowledge, the individual is predicted to have increased risk of recurrence, as compared to an individual whose cancer does not exhibit LOH of the HLA gene.
Embodiment 77. The method of any one of embodiments 67-76, wherein LOH of the HLA gene is determined by;
a) receiving, at one or more processors, an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule;
b) receiving, at one or more processors, a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles;
c) determining, by the one or more processors, an objective function that measures a difference between the relative binding propensity and the observed allele frequency of the HLA allele;
d) determining, by the one or more processors, an optimization model configured to minimize the objective function;
e) determining, by the one or more processors, an adjusted allele frequency of the HLA allele based on the optimization model and the observed allele frequency; and
f) determining, by the one or more processors, that LOH has occurred when the adjusted allele frequency of the HLA allele is less than a predetermined threshold.
   Embodiment 78. A method of treating or delaying progression of cancer, comprising:

(1) detecting loss-of-heterozygosity (LOH) of a human leukocyte antigen (HLA) gene in a sample obtained from an individual, wherein LOH of the HLA gene is detected by:
a) receiving, at one or more processors, an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule;
b) receiving, at one or more processors, a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles;
c) executing, by the one or more processors, an objective function to measure a difference between the relative binding propensity and the observed allele frequency of the HLA allele;
d) executing, by the one or more processors, an optimization model to minimize the objective function;
e) determining, by the one or more processors, an adjusted allele frequency of the HLA allele based on the optimization model and the observed allele frequency; and
f) determining, by the one or more processors, that LOH has occurred when the adjusted allele frequency of the HLA allele is less than a predetermined threshold; and

(2) based at least in part on detection of LOH of the HLA gene, administering an effective amount of a treatment other than an immune checkpoint inhibitor (ICI) to the individual.

Embodiment 79. A method of treating or delaying progression of cancer, comprising:

(1) detecting lack of loss-of-heterozygosity (LOH) of a human leukocyte antigen (HLA) gene in a sample obtained from an individual, wherein lack of LOH of the HLA gene is detected by:
a) receiving, at one or more processors, an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule;
b) receiving, at one or more processors, a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles;
c) executing, by the one or more processors, an objective function to measure a difference between the relative binding propensity and the observed allele frequency of the HLA allele;
d) executing, by the one or more processors, an optimization model to minimize the objective function;
e) determining, by the one or more processors, an adjusted allele frequency of the HLA allele based on the optimization model and the observed allele frequency; and
f) determining, by the one or more processors, that LOH has not occurred when the adjusted allele frequency of the HLA allele is greater than a predetermined threshold; and

(2) based at least in part on detection of lack of LOH of the HLA gene, administering an effective amount of an immune checkpoint inhibitor (ICI) to the individual.

Embodiment 80. The method of any one of embodiments 67-79, wherein the ICT comprises a PD-1 inhibitor, a PD-L1 inhibitor, or a CTLA-4 inhibitor.
Embodiment 81. The method of any one of embodiments 67-80, wherein the method further comprises detecting a tumor mutation burden (TMB) in a sample obtained from the individual.
Embodiment 82. The method of any one of embodiments 67-80, wherein the method further comprises acquiring knowledge of a tumor mutation burden (TMB) in a sample obtained from the individual.
Embodiment 83. The method of any one of embodiments 67-82, wherein the treatment or the one or more treatment options further comprise a second therapeutic agent.
Embodiment 84. The method of any one of embodiments 67-69 and 72-83, wherein LOH of the HLA gene and high TMB in the sample indicate that the individual is likely to benefit from a treatment comprising an immune checkpoint inhibitor (ICI).
Embodiment 85. The method of embodiment 84, wherein the one or more treatment options include treatment comprising an ICI.
Embodiment 86. The method of any one of embodiments 81-85, wherein LOH of the HLA gene and high TMB are detected in the same sample obtained from the individual.
Embodiment 87. The method of any one of embodiments 81-85, wherein LOH of the HLA gene and high TMB are detected in different samples obtained from the individual.
Embodiment 88. A method of selecting treatment for an individual having cancer, comprising (a) acquiring knowledge of loss-of-heterozygosity (LOH) of a human leukocyte antigen (HLA) gene in a sample from an individual having cancer, wherein LOH of the HLA gene is detected according to the method of any one of embodiments 36-66; and (b) acquiring knowledge of high tumor mutational burden (TMB) in a sample from the individual having cancer; wherein responsive to the acquisition of said knowledge in (a) and (b): (i) the individual is classified as a candidate to receive treatment with an immune checkpoint inhibitor (ICI); (ii) the individual is identified as likely to respond to a treatment that comprises an immune checkpoint inhibitor (ICI); and/or (iii) the individual is classified as a candidate to receive a treatment comprising an immune checkpoint inhibitor (ICI).
Embodiment 89. A method of predicting survival of an individual having cancer treated with an immune checkpoint inhibitor (ICI), comprising: (a) acquiring knowledge of loss-of-heterozygosity (LOH) of a human leukocyte antigen (HLA) gene in a sample from the individual, wherein LOH of the HLA gene is detected according to the method of any one of embodiments 36-66, (b) acquiring knowledge of high tumor mutational burden (TMB) in a sample from the individual; wherein responsive to the acquisition of said knowledge in (a) and (b), the individual is predicted to have longer survival after treatment with the ICI, as compared to survival of an individual treated with the ICI whose cancer has LOH of an HLA gene without a high TMB.
Embodiment 90. A method of treating or delaying progression of cancer, comprising:

(1) detecting loss-of-heterozygosity (LOH) of a human leukocyte antigen (HLA) gene in a sample obtained from an individual, wherein LOH of the HLA gene is detected by:
a) receiving, at one or more processors, an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule;
b) receiving, at one or more processors, a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles;
c) executing, by the one or more processors, an objective function to measure a difference between the relative binding propensity and the observed allele frequency of the HLA allele;
d) executing, by the one or more processors, an optimization model to minimize the objective function;
e) determining, by the one or more processors, an adjusted allele frequency of the HLA allele based on the optimization model and the observed allele frequency; and
f) determining, by the one or more processors, that LOH has occurred when the adjusted allele frequency of the HLA allele is less than a predetermined threshold;

(2) detecting high tumor mutational burden (TMB) in a sample obtained from the individual; and

(3) based at least in part on detection of LOH of the HLA gene and high TMB, administering an effective amount of a treatment comprising an immune checkpoint inhibitor (ICI) to the individual.

Embodiment 91. A non-transitory computer readable storage medium comprising one or more programs executable by one or more computer processors for performing a method, comprising:

identifying, using the one or more processors, a plurality of chemical reactions such that:
each reaction corresponds to a bait molecule binding to a different allele of a polymorphic gene, and each reaction resulting in capture of a corresponding allele fraction;
the plurality of chemical reactions consists of a first subset of reactions and a second subset of reactions, in which the first and second subsets share no reaction in common and in which the first and second subsets each comprise at least one chemical reaction;

identifying, using the one or more processors, a plurality of equations that collectively relate binding propensities of each chemical reaction and allele fraction of each captured allele;

receiving, at the one or more processors, empirically identified relative binding propensities of the first subset of the plurality of chemical reactions; and

identifying, using the one or more processors, the relative binding propensities of the second subset by minimizing a total error.

Embodiment 92. The non-transitory computer readable storage medium of embodiment 91, wherein minimizing the total error is subject to a constraint that a median relative binding propensity is equal to 1.
Embodiment 93. The non-transitory computer readable storage medium of embodiment 91, wherein one relative binding propensity is set equal to 1.
Embodiment 94. The non-transitory computer readable storage medium of embodiment 91, wherein minimizing the total error includes performing a least squares procedure.
Embodiment 95. The non-transitory computer readable storage medium of embodiment 91, wherein the method further comprises:

receiving, at the one or more processors, measured raw allele frequencies in a DNA sample of a patient, wherein the measured raw allele frequencies were measured by performing a hybrid capture process; and

scaling, at the one or more processors, the measured raw allele frequencies using the first and second subsets of relative binding propensities, thereby mitigating sampling bias.

Embodiment 96. The non-transitory computer readable storage medium of embodiment 91, wherein the polymorphic gene includes a Human Leukocyte Antigen gene.
Embodiment 97. The non-transitory computer readable storage medium of embodiment 91, wherein the polymorphic gene is ST7/RAY1, ARH1/NOEY2, TSLC1, RB, PTEN, SMAD2, SMAD4, DCC, TP53, ATM, miR-15a, miR-16-1, NAT2, BRCA1, BRCA2, hOGG1, CDH1, IGF2, CDKN1C/P57, MEN1, PRKAR1A, H19, KRAS, BAP1, PTCH1, SMO, SUFU, NOTCH1, PPP6C, LATS1, CASP8, PTPN14, ARID1A, FBXW7, M6P/IGF2R, IFN-alpha, an olfactory receptor gene, CBFA2T3, DUTT1, FHIT, APC, P16, FCMD, TSC2, miR-34, c-MPL, RUNX3, DIRAS3, NRAS, miR-9, FAM50B, PLAGL1, ER, FLT3, ZDBF2, GPR1, c-KIT, NAP1L5, GRB10, EGFR, PEG10, BRAF, MEST, JAK2, DAPK1, LIT1, WT1, NF-1, PR, c-CBL, DLK1, AKT1, SNURF, a cytochrome P450 gene (CYP), ZNF587, SOCS1, TIMP2, RUNX1, AR, CEBPA, C19MC, EMP3, ZNF331, CDKN2A, PEG3, NNAT, GNAS, or GATA5.
Embodiment 98. The non-transitory computer readable storage medium of embodiment 95, wherein the method further comprises determining, at the one or more processors, whether the patient has experienced a loss of heterozygosity.
Embodiment 99. An immune checkpoint inhibitor (ICI) for use in method of treating or delaying progression of cancer in an individual, wherein lack of loss-of-heterozygosity (LOH) of a human leukocyte antigen (HLA) gene has been detected in a sample obtained from the individual by:
a) receiving, at one or more processors, an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule;
b) receiving, at one or more processors, a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles;
c) executing, by the one or more processors, an objective function to measure a difference between the relative binding propensity and the observed allele frequency of the HLA allele;
d) executing, by the one or more processors, an optimization model to minimize the objective function;
e) determining, by the one or more processors, an adjusted allele frequency of the HLA allele based on the optimization model and the observed allele frequency; and
f) determining, by the one or more processors, that LOH has not occurred when the adjusted allele frequency of the HLA allele is greater than a predetermined threshold.
   Embodiment 100. An immune checkpoint inhibitor (ICI) for use in method of treating or delaying progression of cancer in an individual, wherein loss-of-heterozygosity (LOH) of a human leukocyte antigen (HLA) gene and high tumor mutational burden (TMB) have been detected in sample(s) obtained from the individual by:
a) receiving, at one or more processors, an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule;
b) receiving, at one or more processors, a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles;
c) executing, by the one or more processors, an objective function to measure a difference between the relative binding propensity and the observed allele frequency of the HLA allele;
d) executing, by the one or more processors, an optimization model to minimize the objective function;
e) determining, by the one or more processors, an adjusted allele frequency of the HLA allele based on the optimization model and the observed allele frequency;
f) determining, by the one or more processors, that LOH has occurred when the adjusted allele frequency of the HLA allele is less than a predetermined threshold; and
g) acquiring knowledge of, or detecting, a high TMB in a sample obtained from the individual.
   Embodiment 101. An immune checkpoint inhibitor (ICI) for use in manufacture of a medicament for treating or delaying progression of cancer in an individual, wherein loss-of-heterozygosity (LOH) of a human leukocyte antigen (HLA) gene and high tumor mutational burden (TMB) have been detected in a sample obtained from the individual by:
a) receiving, at one or more processors, an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule;
b) receiving, at one or more processors, a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles;
c) executing, by the one or more processors, an objective function to measure a difference between the relative binding propensity and the observed allele frequency of the HLA allele;
d) executing, by the one or more processors, an optimization model to minimize the objective function;
e) determining, by the one or more processors, an adjusted allele frequency of the HLA allele based on the optimization model and the observed allele frequency; and
f) determining, by the one or more processors, that LOH has occurred when the adjusted allele frequency of the HLA allele is less than a predetermined threshold; and
g) acquiring knowledge of, or detecting, a high TMB in a sample obtained from the individual.
   Embodiment 102. An immune checkpoint inhibitor (ICI) for use in manufacture of a medicament for treating or delaying progression of cancer in an individual, wherein lack of loss-of-heterozygosity (LOH) of a human leukocyte antigen (HLA) gene has been detected in a sample obtained from the individual by:
a) receiving, at one or more processors, an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule;
b) receiving, at one or more processors, a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles;
c) executing, by the one or more processors, an objective function to measure a difference between the relative binding propensity and the observed allele frequency of the HLA allele;
d) executing, by the one or more processors, an optimization model to minimize the objective function;
e) determining, by the one or more processors, an adjusted allele frequency of the HLA allele based on the optimization model and the observed allele frequency; and
f) determining, by the one or more processors, that LOH has not occurred when the adjusted allele frequency of the HLA allele is greater than a predetermined threshold.
   Embodiment 103. A system, comprising:

one or more processors; and

a memory configured to store one or more computer program instructions, wherein the one or more computer program instructions when executed by the one or more processors are configured to:
identify a plurality of chemical reactions such that:
   each reaction corresponds to a bait molecule binding to a different allele of a polymorphic gene, and each reaction resulting in capture of a corresponding allele fraction;
   the plurality of chemical reactions consists of a first subset of reactions and a second subset of reactions, in which the first and second subsets share no reaction in common and in which the first and second subsets each comprise at least one chemical reaction;
identify a plurality of equations that collectively relate binding propensities of each chemical reaction and allele fraction of each captured allele;
receive empirically identified relative binding propensities of the first subset of the plurality of chemical reactions; and
identify the relative binding propensities of the second subset by minimizing a total error.
   Embodiment 104. The system of embodiment 103, wherein minimizing the total error is subject to a constraint that a median relative binding propensity is equal to 1.
   Embodiment 105. The system of embodiment 103, wherein one relative binding propensity is set equal to 1.
   Embodiment 106. The system of embodiment 103, wherein minimizing the total error includes performing a least squares procedure.
   Embodiment 107. The system of embodiment 103, wherein the one or more computer program instructions when executed by the one or more processors are further configured to:

receive measured raw allele frequencies in a DNA sample of a patient, wherein the measured raw allele frequencies were measured by performing a hybrid capture process; and

scale the measured raw allele frequencies using the first and second subsets of relative binding propensities, thereby mitigating sampling bias.

Embodiment 108. The system of embodiment 103, wherein the polymorphic gene includes a Human Leukocyte Antigen gene.
Embodiment 109. The system of embodiment 103, wherein the polymorphic gene is ST7/RAY1, ARH1/NOEY2, TSLC1, RB, PTEN, SMAD2, SMAD4, DCC, TP53, ATM, miR-15a, miR-16-1, NAT2, BRCA1, BRCA2, hOGG1, CDH1, IGF2, CDKN1C/P57, MEN1, PRKAR1A, H19, KRAS, BAP1, PTCH1, SMO, SUFU, NOTCH1, PPP6C, LATS1, CASP8, PTPN14, ARID1A, FBXW7, M6P/TGF2R, IFN-alpha, an olfactory receptor gene, CBFA2T3, DUTT1, FHIT, APC, P16, FCMD, TSC2, miR-34, c-MPL, RUNX3, DIRAS3, NRAS, miR-9, FAM50B, PLAGL1, ER, FLT3, ZDBF2, GPR1, c-KIT, NAP1L5, GRB10, EGFR, PEG10, BRAF, MEST, JAK2, DAPK1, LIT1, WT1, NF-1, PR, c-CBL, DLK1, AKT1, SNURF, a cytochrome P450 gene (CYP), ZNF587, SOCS1, TIMP2, RUNX1, AR, CEBPA, C19MC, EMP3, ZNF331, CDKN2A, PEG3, NNAT, GNAS, or GATA5.
Embodiment 110. The system of embodiment 107, wherein the one or more computer program instructions when executed by the one or more processors are further configured to determine whether the patient has experienced a loss of heterozygosity.
Embodiment 111. A non-transitory computer readable storage medium comprising one or more programs executable by one or more computer processors for performing a method, comprising:

receiving, using the one or more processors, an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule;

receiving, using the one or more processors, a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles;

executing, using the one or more processors, an objective function to measure a difference between the relative binding propensity and the observed allele frequency of the HLA allele;

executing, using the one or more processors, an optimization model to minimize the objective function;

determining, using the one or more processors, an adjusted allele frequency of the HLA allele based on the optimization model and the observed allele frequency; and

determining, using the one or more processors, that LOH has occurred when the adjusted allele frequency of the HLA allele is less than a predetermined threshold.

Embodiment 112. The non-transitory computer readable storage medium of embodiment 111, wherein the HLA gene is a human HLA-A, HLA-B, or HLA-C gene.
Embodiment 113. The non-transitory computer readable storage medium of embodiment 111 or embodiment 112, wherein the plurality of sequence reads was obtained by sequencing nucleic acids obtained from a sample comprising tumor cells and/or tumor nucleic acids.
Embodiment 114. The non-transitory computer readable storage medium of embodiment 113, wherein the sample further comprises non-tumor cells.
Embodiment 115. The non-transitory computer readable storage medium of embodiment 113, wherein the sample is from a tumor biopsy or tumor specimen.
Embodiment 116. The non-transitory computer readable storage medium of embodiment 113, wherein the sample comprises tumor cell-free DNA (cfDNA).
Embodiment 117. The non-transitory computer readable storage medium of embodiment 113, wherein the sample comprises fluid, cells, or tissue.
Embodiment 118. The non-transitory computer readable storage medium of embodiment 117, wherein the sample comprises blood or plasma.
Embodiment 119. The non-transitory computer readable storage medium of embodiment 113, wherein the sample comprises a tumor biopsy or a circulating tumor cell.
Embodiment 120. The non-transitory computer readable storage medium of embodiment 113, wherein the sample is a nucleic acid sample.
Embodiment 121. The non-transitory computer readable storage medium of embodiment 120, wherein the nucleic acid sample comprises mRNA, genomic DNA, circulating tumor DNA, cell-free DNA, or cell-free RNA.
Embodiment 122. The non-transitory computer readable storage medium of any one of embodiments 111-121, wherein the method further comprises:

using the one or more processors, determining a tumor mutational burden (TMB) from a plurality of sequence reads, wherein the plurality of sequence reads was obtained by sequencing nucleic acids at least a portion of a genome.

Embodiment 123. The non-transitory computer readable storage medium of embodiment 122, wherein the TMB is determined based on a number of non-driver somatic coding mutations per megabase of genome sequenced.
Embodiment 124. A system, comprising:

one or more processors; and

a memory configured to store one or more computer program instructions, wherein the one or more computer program instructions when executed by the one or more processors are configured to:
determine an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule;
determine a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles;
execute an objective function to measure a difference between the relative binding propensity and the observed allele frequency of the HLA allele;
execute an optimization model to minimize the objective function;
determine an adjusted allele frequency of the HLA allele based on the optimization model and the observed allele frequency; and
determine that LOH has occurred when the adjusted allele frequency of the HLA allele is less than a predetermined threshold.
   Embodiment 125. The system of embodiment 124, wherein the HLA gene is a human HLA-A, HILA-B, or HLA-C gene.
   Embodiment 126. The system of embodiment 124 or embodiment 125, wherein the plurality of sequence reads was obtained by sequencing nucleic acids obtained from a sample comprising tumor cells and/or tumor nucleic acids.
   Embodiment 127. The system of embodiment 126, wherein the sample further comprises non-tumor cells.
   Embodiment 128. The system of embodiment 126, wherein the sample is from a tumor biopsy or tumor specimen.
   Embodiment 129. The system of embodiment 126, wherein the sample comprises tumor cell-free DNA (cfDNA).
   Embodiment 130. The system of embodiment 126, wherein the sample comprises fluid, cells, or tissue.
   Embodiment 131. The system of embodiment 130, wherein the sample comprises blood or plasma.
   Embodiment 132. The system of embodiment 126, wherein the sample comprises a tumor biopsy or a circulating tumor cell.
   Embodiment 133. The system of embodiment 126, wherein the sample is a nucleic acid sample.
   Embodiment 134. The system of embodiment 133, wherein the nucleic acid sample comprises mRNA, genomic DNA, circulating tumor DNA, cell-free DNA, or cell-free RNA.
   Embodiment 135. The system of any one of embodiments 124-134, wherein the one or more computer program instructions when executed by the one or more processors are further configured to:

using the one or more processors, acquiring knowledge of or detecting tumor mutational burden (TMB) from a plurality of sequence reads, wherein the plurality of sequence reads was obtained by sequencing nucleic acids at least a portion of a genome.

Embodiment 136. The system of embodiment 135, wherein the TMB is determined based on a number of non-driver somatic coding mutations per megabase of genome sequenced.

The method steps of the invention(s) described herein are intended to include any suitable method of causing one or more other parties or entities to perform the steps, unless a different meaning is expressly provided or otherwise clear from the context. Such parties or entities need not be under the direction or control of any other party or entity, and need not be located within a particular jurisdiction. Thus, for example, a description or recitation of “adding a first number to a second number” includes causing one or more parties or entities to add the two numbers together. For example, if person X engages in an arm's length transaction with person Y to add the two numbers, and person Y indeed adds the two numbers, then both persons X and Y perform the step as recited: person Y by virtue of the fact that he actually added the numbers, and person X by virtue of the fact that he caused person Y to add the numbers. Furthermore, if person X is located within the United States and person Y is located outside the United States, then the method is performed in the United States by virtue of person X's participation in causing the step to be performed.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The disclosures of all publications, patents, and patent applications referred to herein are each hereby incorporated by reference in their entireties. To the extent that any reference incorporated by reference conflicts with the instant disclosure, the instant disclosure shall control.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Somatic HLA Class I Loss is a Widespread Mechanism of Cancer Immune Evasion which Refines the Use of Tumor Mutational Burden as a Biomarker of Checkpoint Inhibitor Response

This example describes the results from experiments designed to predict patient survival in ICI-treated non-small cell lung cancer (NSCLC) from somatic HLA-I LOH. This example also describes experiments to determine the incidence of HLA-I LOH across cancer types and in tumors with high tumor mutational burden (TMB).

Immune checkpoint inhibitors (ICIs) have revolutionized current treatments for advanced cancer patients and are thought to reinvigorate the patient's own T-cell mediated immune response [1-5]. CD8 T-cells recognize tumor cells via the presentation of tumor-specific mutant peptides (neoantigens) on human leukocyte antigen class I (HLA-I)-encoded major histocompatibility complex class I (MHC-T) proteins [6-8]. This hypothesis is supported by the efficacy of ICTs in diseases with a high tumor mutational burden (TMB) and the potential pan-cancer utility of TMB as a biomarker of checkpoint inhibitor response. Yet, in trials focused only on non-small cell lung cancer (NSCLC), TMB fails to sufficiently predict for patient survival [2,5,9]. However, efforts to use HLA genotyping to predict the relative efficiency of neoantigen presentation and use of this information together with TMB to predict checkpoint responses are showing promise (Goodman A M, et al. Genome Med. 2020; 12(1):45; Shim J H, et al. Ann Oncol. 2020; 31(7):902-11).

The loss of HLA-I can result in fewer neoantigens presented to immune cells and can lead to immune escape, as predicted in FIG. 4A. The relation between HLA-I LOH and the immune response is further illustrated in FIG. 4B. HLA-I LOH is related to TMB through neoantigens and to PD-L1 as an evasion mechanism. In this example, somatic loss of HLA-I was shown to be a negative predictor of patient survival in ICI-treated NSCLC, which blunts the effect of high TMB. The landscape of somatic HLA-I LOH in over 83,000 patient samples across 59 disease groups was also determined, finding a pan-cancer incidence of 17% and significant enrichment in tumors with high TMB and inflamed tumors as represented by PD-L1 expression. Combined, TMB and HLA-I LOH may better select patients most likely to benefit from ICI in inflamed cancers and has implications for the design of personalized cancer vaccines.

### Materials and Methods

Genomic Profiling

Genomic data was collected as part of routine clinical care for 83,664 patients using a targeted comprehensive genomic profiling assay in a Clinical Laboratory Improvement Amendments (CLIA)-certified, College of American Pathologists (CAP)-accredited, New York State approved laboratory, as previously described [20]. DNA was extracted and hybrid capture for all coding exons of 315 genes plus 28 introns frequently rearranged in cancer was performed. Libraries were sequenced to a median coverage depth of >500×. Analysis for genomic alterations, including short variant alterations (base substitutions, insertions, and deletions), copy number alterations (amplifications and homozygous deletions), as well as gene rearrangements was performed as previously described [20,21]. TMB was defined as the number of non-driver somatic coding mutations per megabase of genome sequenced [22]. Mutational signatures were determined in samples with ≥20 non-driver somatic missense mutations, including silent and noncoding alterations. Signatures were assigned using the COSMIC signatures of mutational processes in human cancer, as previously described by Zehir et al [23]. A positive status was determined if a sample had ≥40% fit to a mutational process. Viral DNA detection was performed through Velvet [24] de novo assembly of sequencing reads left unmapped to the human reference genome (hg19). Assembled contigs were competitively mapped by BLASTn (BLAST+v2.6.0 [25]) to the NCBI database of >3 million viral nucleotide sequences and a positive viral status was determined by contigs ≥80 nucleotides in length with ≥97% identity to the BLAST sequence.

Histology

PD-L1 status was determined through immunohistochemistry performed on formalin-fixed paraffin-embedded (FFPE) tissue sections, with the use of the commercially available antibody clones 22C3 (Dako/Agilent, Santa Clara, Calif., USA) or SP142 (Ventana, Tucson, Ariz., USA). A pathologist determined the percent of tumor cells with expression (0%-100%) and the intensity of expression (0, 1+, 2+). PD-L1 expression was reported as a continuous variable with the percentage of tumor cells staining with ≥1+ intensity. PD-L1 expression for each sample was also summarized as negative (<1% tumor cells) or positive (≥1% tumor cells). The pathology laboratory established performance characteristics for this assay per the requirements of the Clinical Laboratory Improvement Amendments (CLIA '88) and in accordance with College of American Pathologists (CAP) checklist requirements and guidance. Estrogen receptor (ER) and progesterone receptor (PR) status were manually abstracted from pathology reports or by an automated machine-reading algorithm validated to 97% accuracy for ER and 94% accuracy for PR. Genomic amplification status from next-generation sequencing (NGS) was used for HER2 (ERBB2) positivity [20].

HLA Loss of Heterozygosity Determination and Neoantigen Prediction

HLA-I zygosity was determined using the SGZ (somatic-germline-zygosity) algorithm, a computational method previously described by Sun et al. [21] for zygosity prediction from next-generation sequencing results of mixed tumor-normal samples (20-95% tumor). In brief, SGZ models zygosity by taking into account the tumor purity, tumor ploidy, minor allele frequency, and local copy number of each genomic segment. The minor allele frequency of each HLA-I gene (HLA-A, -B, and -C) was calculated separately. HLA-I genotyping of sequencing results was performed by OptiType [26] v1.3.1, to a four-digit resolution. HLA reference sequences that matched the germline alleles for each sample were obtained from the IPD-IMGT/HLA database [27]. Only germline heterozygous alleles were assessed for LOH and samples identified as being germline homozygous at all three loci were not used in this study.

Sequencing reads that aligned to the HLA region of the human reference genome (hg19, 6p21-22) as well as all unmapped reads were extracted using Samtools [28]v1.5. Picard v1.56 was used to remove all PCR and optical duplicates. Reads were competitively re-aligned to the HLA reference sequences specific to each sample using BWA [28] v0.7.17. Samtools v1.5 was used to keep only uniquely aligned reads and to remove all unpaired mates. A local alignment was performed between each germline heterozygous homologous allele using BLAST+v2.6.0. The BTOP function was used to identify mismatch positions between each homologous allele.

Samtools v1.5 was used to collect all unique reads that aligned to each mismatch position and the allele frequency for each allele was calculated as the number of reads that uniquely aligned to one homologous allele divided by the total number of uniquely aligned reads to both homologous alleles. A baited sequencing method was used to isolate regions of interest. For the HLA-I locus, it was observed that homologous HLA pairs had a consistent allele frequency (AF) skew (FIG. 4E). To account for effects of hybridization on baiting efficiency due to HLA type, the observed AF (obsAF) was modeled as a function of AF and an association constant of an HLA type for the baits.

$${obsAF}_{i , j} = \frac{{k}_{i} \times {AF}_{i , j}}{{k}_{i} \times {AF}_{i , j} + {k}_{j} \times {AF}_{j , i}}$$

where obsAF_{i,j} is the observed allele frequency of HLA type i in the pair i,j; kᵢ is the association constant of HLA type i, and AF_{i,j} is the allele frequency of HLA type i in the sample with pair i,j. Note AF_{i,j}=1−AF_{j,i}.

To fit the association constants, allelic balance was assumed (AF_{i,j}=1−AF_{j,i}). Given that most samples are not under LOH, the median allele frequency of all samples was used with the same pair of homologous HLA types. The constraint that 50 samples were required to provide a representative AF was also added. Thus,

$${obsAF}_{i , j} = \frac{{k}_{i}}{{k}_{i} + {k}_{j}}$$

$${obsAF}_{j , i} = \frac{{k}_{j}}{{k}_{i} + {k}_{j}}$$

Combining these equations yields:

$$\frac{{obsAF}_{i , j}}{{obsAF}_{j , i}} = \frac{{k}_{i}}{{k}_{j}}$$

To determine the best k values for each HLA type, least squares fitting was used:

$$min \sum {( \frac{{obsAF}_{i , j}}{{obsAF}_{j , i}} - \frac{{k}_{i}}{{k}_{j}} )}^{2}$$

for all pairs

With the k values determined, the input allele frequency can be determined from the observed allele frequency:

$${AF}_{i , j} = \frac{{k}_{j} \times {obsAF}_{i , j}}{{k}_{i} + {k}_{j} \times {obsAF}_{i , j} - {k}_{i} \times {obsAF}_{i , j}}$$

It was assessed how the association constants (k values) mapped to sequence diversity in HLA-A. A dendrogram of HLA-A two-digit sequences exhibited two major branches with one branch having k values >1 and the other having k values <1, supporting the hypothesis that sequence-driven hybridization effects were the underlying cause of MAF skewing (FIG. 4F). The sequence of the bait molecule is depicted in the dendrogram shown in FIG. 4G. HLA haplotypes which have diverged the most from the baited sequence have worse binding than haplotypes more closely related to the bait sequence.

Using this model, adjusted minor allele frequencies, representing the true allele frequency in the sample, were calculated from the observed minor allele frequencies. The adjusted minor allele frequency was then used in the SGZ algorithm described above. At loci identified as having HLA-I LOH, the allele with the lower allele frequency was determined to be the allele under LOH.

Neoantigen Prediction

End-to-end processing and MHC-I binding predictions were calculated using MHCpan-4.0 and the IEDB API for all wild-type and mutant peptides [14]. The API produces proteasomal cleavage scores, TAP transport scores, and MHC-I binding affinities, as well as a total score that combines these aforementioned values in an HLA-to-peptide specific manner. Total scores of at least −0.8 and MHC-I binding affinities of at most 500 nM were used to dichotomize each peptide as a binder or a non-binder in a given sample. Upon identification of binders, binder mutant peptides were filtered against their wild-type counterpart.

Clinical Cohort and Survival Analysis

The retrospective clinical analysis utilized a real-world clinico-genomic dataset (data collected through Jun. 30, 2019) which includes electronic health record (EHR) data for patients in the database who underwent comprehensive genomic profiling [11]. The de-identified patient-level clinical data from the EHR included structured data (eg. treatment prescribed, treatment received, treatment start date) in addition to unstructured data (eg. smoking status, histology) collected via technology-enabled chart abstraction from physician's notes by trained medical record abstractors who followed pre-specified, standardized policies and procedures. De-identified patient-level genomic data included specimen (eg. tumor mutational burden, pathological tumor purity) and genomic (eg. gene altered, alteration type) data reported by comprehensive genomic profiling.

The patients included in the clinical analysis were diagnosed with non-squamous NSCLC and negative for EGFR and ALK alterations. A variety of second-line ICI monotherapies were received, including nivolumab, pembrolizumab, durvalumab and atezolizumab. The primary clinical end point studied was overall survival, from start of second-line ICI regimen until death or loss of follow-up. In the survival analyses, to account for left truncation, patients were treated as at risk of death only after the later of their sequencing report date and their second visit in the Flatiron network on or after Jan. 1, 2011, as both are requirements for inclusion in the cohort. For the Kaplan-Meier analyses, the log-rank test was used to compare groups. Significance of survival outcomes in this cohort were not affected when adjusted for race/ethnicity, age at the start of second-line ICI, first line therapy received, and medical practice type, in a multivariate analysis. Analyses were performed on the R software version 3.6.0 (R Foundation for Statistical Computing).

Patient Consent and Data Availability

Approval for this study, including a waiver of informed consent and a HIPAA waiver of authorization was obtained from the Western Institutional Review Board (Protocol No. 20152817). Patients were not consented for release of raw sequencing data.

### Results

To assess the landscape of allele-specific HLA-I LOH, a pipeline was developed for tumor-only next-generation sequencing of tissue biopsies that can detect loss of heterozygosity (LOH) as well as germline homozygosity at the HLA-I locus (HLA-A, -B, and -C). An overview of this pipeline is depicted in FIG. 4C. Additional methodological considerations for detecting HLA-I LOH are shown in FIG. 4D.

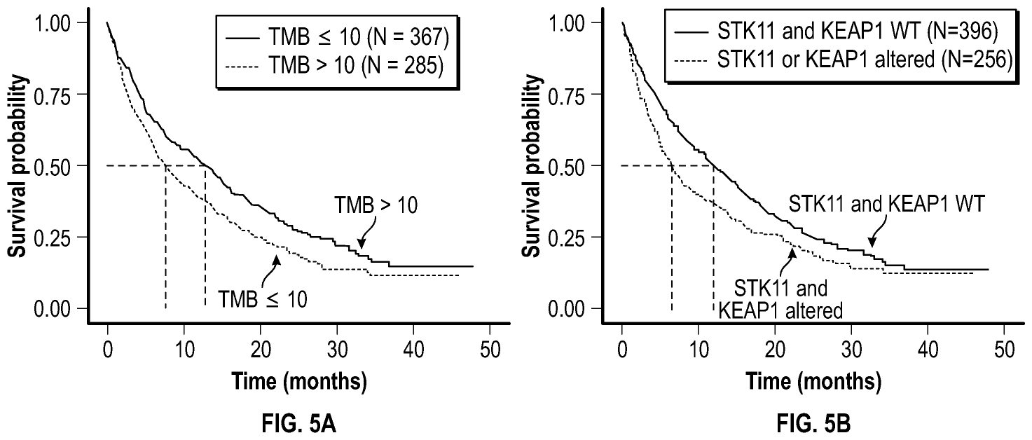

The effect of survival probability of known genomic association in clinic-genomic databases are shown in FIGS. 5A & 5B. TMB high is positively associated with survival (HR=0.76, P=0.007), as shown in FIG. 5A. However, loss of SK11 or KEAP1 was found to be negatively associated with survival (HR=1.3, P=0.009), as shown in FIG. 5B.

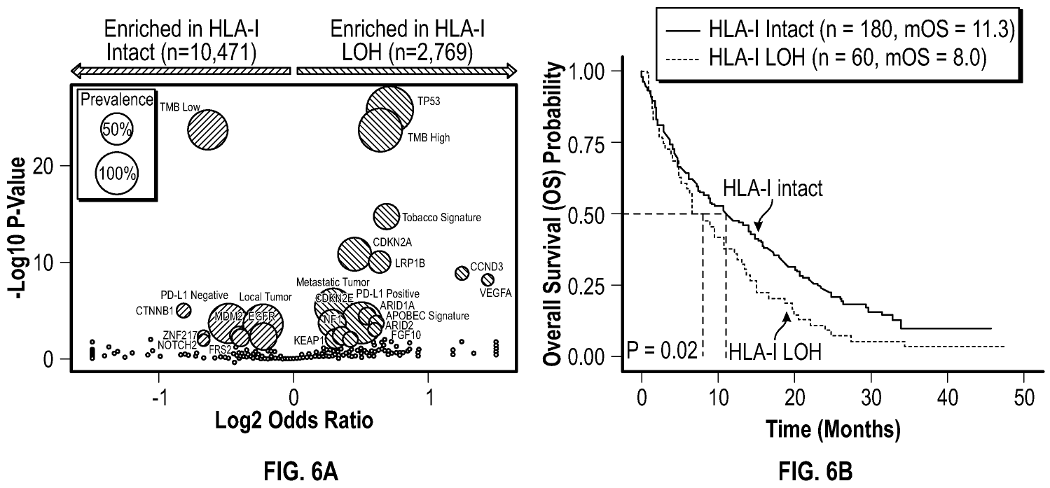

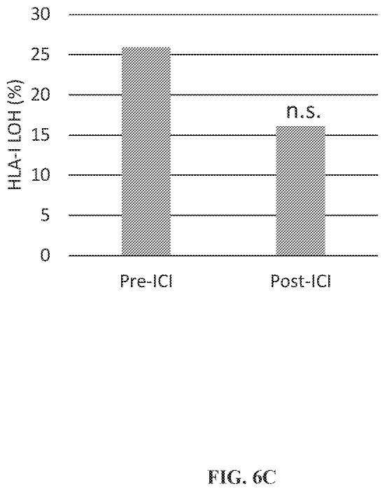

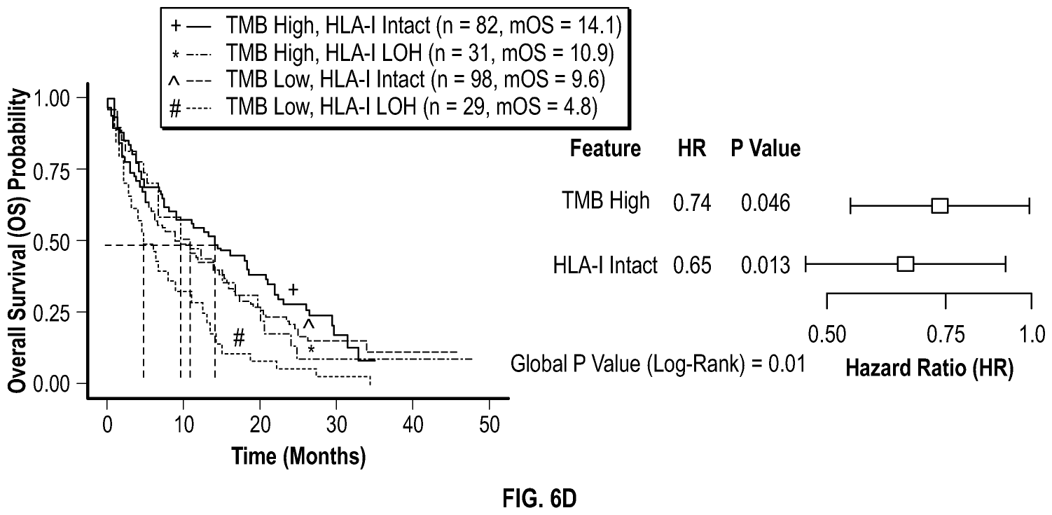

In line with previous reports [10], non-squamous NSCLC with HLA-I LOH classified as somatic LOH of at least one HLA-I allele, were enriched for samples with high TMB (≥10 mutations per megabase [mut/Mb]), PD-L1+(≥1% tumor proportion score), smoking and APOBEC mutational signatures, tumor metastasis, and alterations in TP53, as shown in FIG. 6A. To investigate the impact of HLA-I LOH on ICI treatment, a real-world clinico-genomic dataset [11] was employed to analyze a cohort of 240 patients with EGFR- and ALK-wild-type, non-squamous NSCLC who received second-line ICI monotherapy between July 2014 and February 2019. Table 1 summarizes the characteristics of patients in the real-world clinico-genomic cohort.

TABLE 1
Characteristics of patients in the real-world clinico-genomic cohort

| | | HLA-I Intact | | HLA-I Lost | | P |
|---|---|---|---|---|---|---|
| Characteristic | | (n = 183) | | (n = 60) | | Value |
| Median age at the start of second line ICI therapy, (IQR) | | 68.0 | (60.0-73.5) | 67.5 | (60.8-72.2) | 0.89 |
| Gender | Male | 74 | (41%) | 25 | (42%) | 1.00 |
| | Female | 106 | (59%) | 35 | (58%) | |
| Smoking status | History of smoking | 154 | (86%) | 56 | (93%) | 0.17 |
| | No history of smoking | 26 | (14%) | 4 | (7%) | |
| Race/Ethnicity | Asian | 4 | (2%) | 0 | (0%) | 0.57 |
| (n = 225/243) | Black or African American | 14 | (8%) | 5 | (9%) | 1.00 |
| | Other | 19 | (11%) | 10 | (18%) | 0.25 |
| | White | 129 | (78%) | 41 | (73%) | 0.58 |
| Stage of disease | I | 16 | (9%) | 5 | (9%) | 1.00 |
| at initial diagnosis | II | 9 | (5%) | 0 | (0%) | 0.12 |
| (n = 240/243) | III | 31 | (17%) | 11 | (19%) | 0.84 |
| | IV | 122 | (69%) | 43 | (73%) | 0.62 |
| First line therapy | Anti-VEGF and chemotherapy | 61 | (34%) | 29 | (48%) | 0.06 |
| received | combinations | | | | | |
| | Clinical study drugs | 5 | (3%) | 1 | (2%) | 1.00 |
| | EGFR tyrosine kinase | 3 | (2%) | 1 | (2%) | 1.00 |
| | inhibitors | | | | | |
| | Platinum based chemotherapy | 103 | (57%) | 27 | (45%) | 0.1 |
| | combinations | | | | | |
| | Single agent chemotherapy | 8 | (4%) | 2 | (3%) | 1.00 |
| Timing of tumor | Before 2^{nd} line ICI | 163 | (91%) | 56 | (93%) | 0.61 |
| biopsy | After 2^{nd} line ICI | 17 | (9%) | 4 | (7%) | |
| Tumor mutational | >=10 mut/Mb | 82 | (46%) | 31 | (53%) | 0.46 |
| burden | <10 mut/Mb | 98 | (54%) | 29 | (48%) | |

A second-line ICI treated (ICI naïve) cohort was chosen because patients were presumably treated irrespective of PD-L1 status given contemporaneous FDA approvals [12]. At the initiation of second line ICI, this cohort had a median overall survival (mOS) of 10.8 months, 25% exhibited HLA-I LOH, 59% were female, and the median age was 68 years old. No demographic variables were significantly different when stratifying the cohort by HLA-I LOH, including biopsy timing (P>0.05). Stratification by somatic HLA-I LOH showed significantly decreased survival in the HLA-I LOH group compared to the HLA-I intact group (mOS loss: 8 months [5.2-13.1]; mOS intact: 11.3 months [8.2-15.3]; HR for HLA-I intact=0.68 [0.49-0.95]; P=0.02).). FIG. 6B depicts the results of this analysis. By comparison, in sponsored randomized controlled clinical trials, mOS for all comer second-line non-squamous NSCLC patients treated with ICI was 12.2 months and patients receiving docetaxel in the control arm demonstrated a mOS of 9.4 months [13]. No effect of biopsy timing was observed in CGBD, as shown in FIG. 6C.

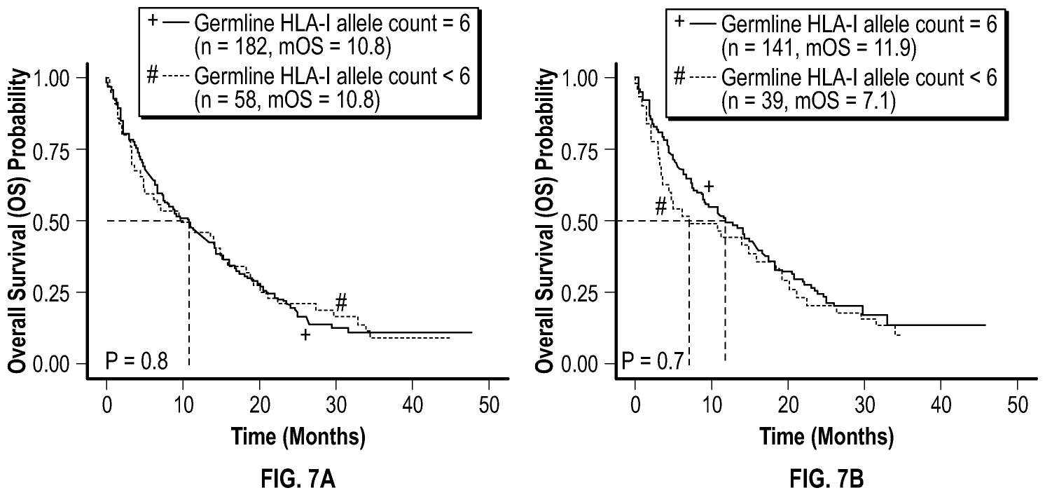

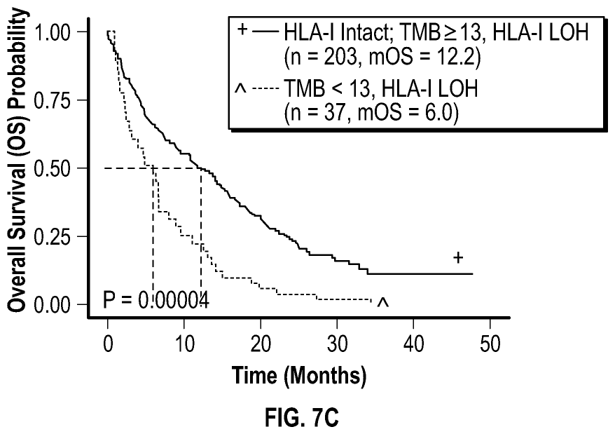

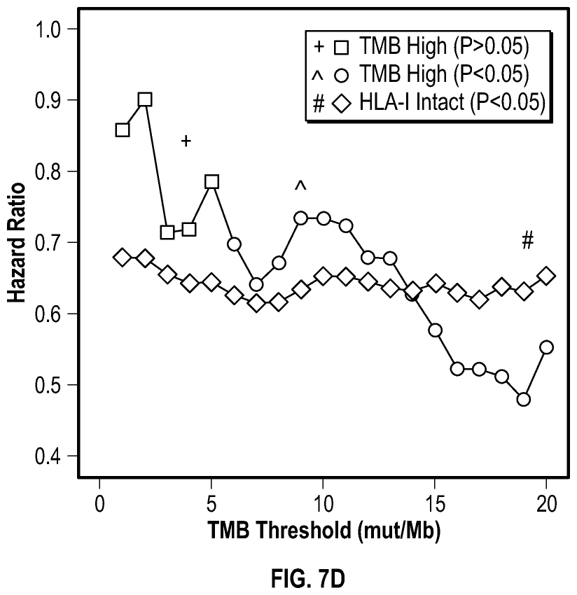

Patients were further stratified by TMB, with a 10 mut/Mb cutoff. The results of this analysis are depicted in FIG. 6D. TMB and HLA-I LOH were independent and significant predictors of survival in a multivariate cox-regression model (HLA-I intact HR=0.65 [0.47-0.91], P=0.01; TMB high HR=0.74 [0.54-0.99], P=0.05). The TMB high, HLA-I intact group had an mOS of 14.09 months [9.0-21.1] while the mOS of the TMB low, HLA-I lost group was 4.83 months [2.86-12.6]. No effect of germline zygosity was found, and no survival difference was observed when stratifying by 6 vs. <6 unique germline HLA-I alleles for either the entire cohort (6 allele HR=1.0 [0.73-1.47], P=0.8), as shown in FIG. 7A, or within the HLA-I intact group (6 allele HR=0.91 [0.60-1.38], P=0.7), as shown in FIG. 7B. To assess whether a combination biomarker could perform better than either HLA-I LOH or TMB alone, the cohort was stratified into two groups by setting different TMB^{hi} thresholds depending on whether the sample was HLA-I intact or HLA-I LOH. The most significant difference was observed when combining all HLA-I intact samples and TMB≥13 mut/Mb for HLA-I LOH (FIG. 7C, HR=0.45 [0.31-0.66], P=0.00004), with 203/240 patients in the combination high group. Using TMB alone to create a predictor with a similar number of biomarker positive patients (200/240) does not significantly stratify survival (FIG. 7D, TMB≥3 mut/Mb, P>0.05). In total, these data show that HLA-I LOH when combined with TMB may identify patients most and least likely to benefit from immune checkpoint inhibitors.

An assessment of 59 different tumor types comprising 83,664 unique patient samples, mostly from tumors of patients with advanced disease, was performed. Table 2 summarizes the results of this analysis.

TABLE 2
Sample counts by tumor

| | Sample | PD-L1 Stained | HLA-I LOH |
|---|---|---|---|
| Tumor Type | Number | Sample Number | Prevalence (%) |
| thymic | 136 | 24 | 41.9 |
| cervical squamous cell carcinoma | 336 | 54 | 39.0 |
| pancreatic islet cell | 254 | 38 | 38.2 |
| adrenocortical carcinoma | 172 | 28 | 36.0 |
| anal squamous cell carcinoma | 236 | 39 | 36.0 |
| penile squamous cell carcinoma | 63 | 13 | 31.7 |
| lung squamous cell carcinoma | 2666 | 842 | 31.3 |
| unknown primary squamous cell carcinoma | 560 | 92 | 28.4 |
| head and neck squamous cell carcinoma | 1134 | 180 | 27.2 |
| skin squamous cell carcinoma | 256 | 46 | 25.4 |
| esophageal | 1967 | 340 | 24.5 |
| vaginal squamous cell carcinoma | 139 | 25 | 24.5 |
| rectal squamous cell carcinoma | 54 | 6 | 24.1 |
| pancreatic | 4049 | 527 | 23.4 |
| renal | 1371 | 228 | 23.0 |
| esophageal squamous cell carcinoma | 324 | 74 | 21.3 |
| cervical | 262 | 45 | 21.0 |
| non-small cell lung carcinoma (NSCLC) | 13240 | 3102 | 20.9 |
| small intestine | 459 | 62 | 20.9 |
| fallopian tube | 377 | 50 | 18.8 |
| biliary | 749 | 120 | 18.7 |
| urinary | 199 | 40 | 18.6 |
| bone sarcoma | 108 | 16 | 18.5 |
| gastric | 1207 | 98 | 18.4 |
| head and neck neuroendocrine | 80 | 12 | 17.5 |
| germ cell | 186 | 26 | 16.7 |
| peritoneal | 292 | 43 | 16.4 |
| unknown primary | 5733 | 905 | 16.3 |
| cholangiocarcinoma | 1542 | 240 | 16.0 |
| bladder | 1299 | 178 | 15.7 |
| ovarian | 4996 | 661 | 15.7 |
| thyroid | 670 | 82 | 15.7 |
| colorectal | 10682 | 1410 | 15.3 |
| appendiceal | 316 | 51 | 13.6 |
| breast | 9686 | 1139 | 13.2 |
| unknown primary neuroendocrine | 644 | 92 | 12.9 |
| soft tissue sarcoma | 638 | 66 | 12.4 |
| mesothelioma | 404 | 70 | 12.4 |
| gastrointestinal neuroendocrine | 269 | 53 | 12.3 |
| skin (other) | 174 | 34 | 12.1 |
| salivary gland | 483 | 65 | 11.4 |
| non-glioma | 716 | 40 | 10.6 |
| head and neck | 231 | 37 | 10.4 |
| carcinoid | 249 | 45 | 9.2 |
| endometrial | 2325 | 347 | 9.1 |
| glioma (non-GBM) | 1519 | 86 | 8.8 |
| gastrointestinal stromal tumor | 442 | 63 | 8.1 |
| glioblastoma (GBM) | 2865 | 219 | 8.1 |
| adenoid cystic carcinoma | 474 | 61 | 6.5 |
| cutaneous melanoma | 1020 | 174 | 6.1 |
| small cell | 1021 | 181 | 5.9 |
| hepatocellular | 570 | 88 | 5.8 |
| prostate | 2774 | 482 | 5.8 |
| uterine | 416 | 60 | 5.5 |
| non-cutaneous melanoma | 222 | 37 | 5.0 |
| Wilms tumor | 54 | 7 | 3.7 |
| prostate neuroendocrine | 110 | 24 | 3.6 |
| Merkel cell carcinoma | 162 | 13 | 3.1 |
| adrenal gland neuroendocrine | 82 | 11 | 2.4 |

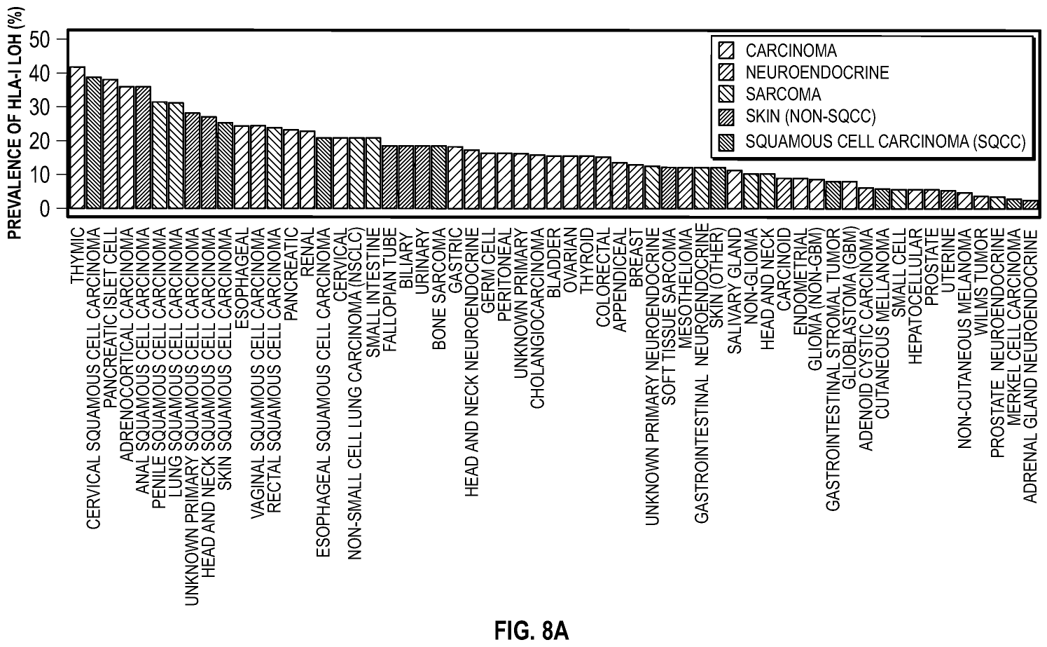

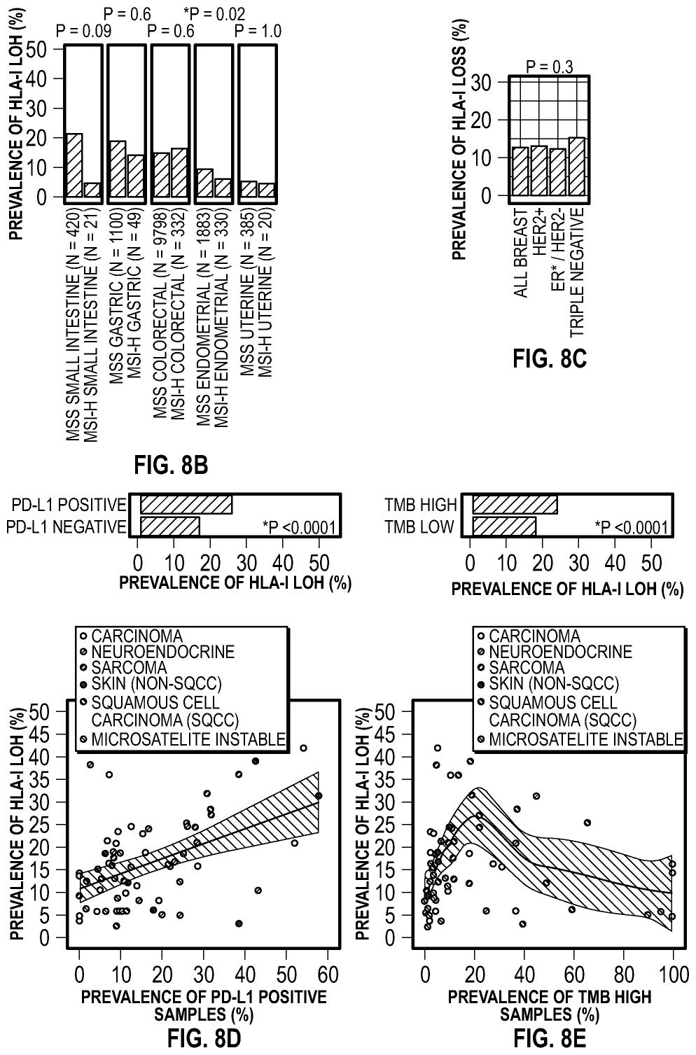

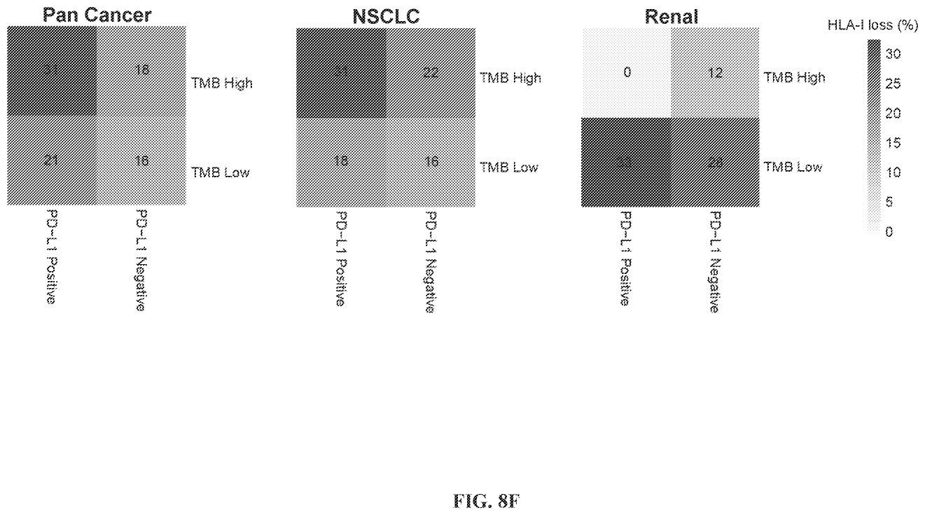

Overall, HLA-I LOH was detected in 17% of solid tumor samples with 85% of HLA-I LOH events involving LOH of the entire HLA-I locus. As shown in FIG. 8A, prevalence varied widely across tumor types (2%-42%). The highest rate of HLA-I LOH was seen in squamous cell carcinomas (SqCCs) (30%), followed by non-SqCC carcinomas (16%), neuroendocrine tumors (11%), sarcomas (11%), and non-SqCC skin cancers (6%). Diseases were further subset by microsatellite instability (MSI) status, finding that HLA-I LOH was either similar in MSI-High and stable (MSS) subsets or increased in MSS tumors with endometrial cancer reaching significance (P=0.02), as shown in FIG. 8B. Subsetting breast cancer by hormone receptor status and HER2 amplification found no significant difference across subsets (P=0.3), as shown in FIG. 8C.

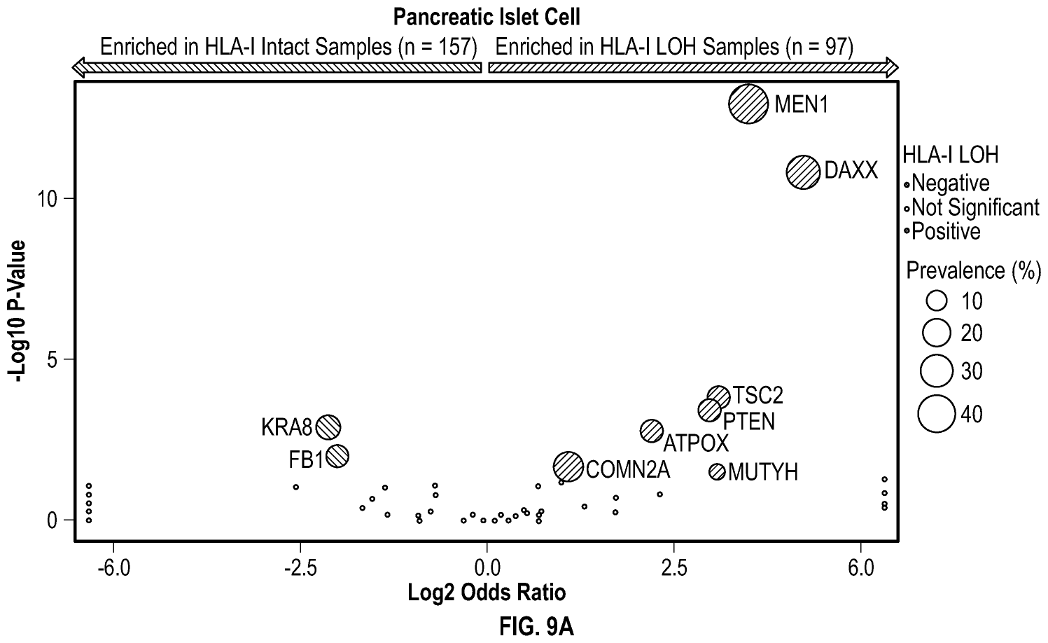

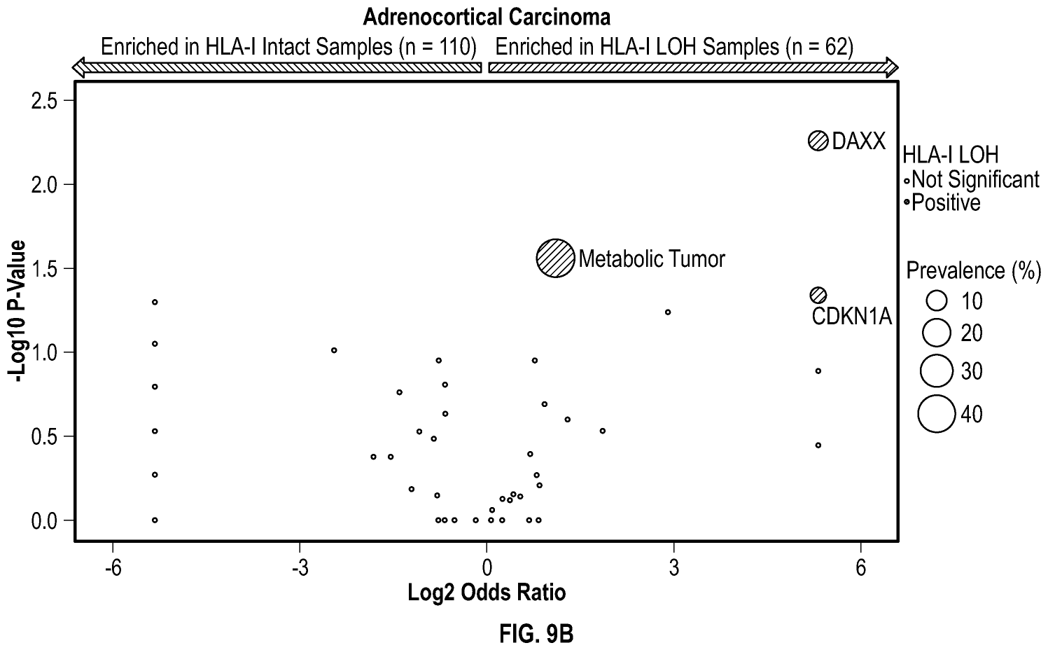

The relationship of HLA-I with PD-L1 and TMB was also examined. As shown in FIG. 8D, a significantly higher prevalence of HLA-I LOH was found in PD-L1⁺ samples (25%) compared to 16% of PD-L1⁻ samples (P<0.0001). HLA-I LOH was also significantly associated with high TMB (TMB high: 21%, TMB low: 16%; P<0.0001), as shown in FIG. 8E. As also shown in FIG. 8D, the incidences of PD-L1⁺ and HLA-I LOH were linearly correlated (P=0.0001). However, as is shown in FIG. 8E, TMB demonstrated a more complex relationship where diseases with the lowest TMB (e.g. neuroendocrine tumors) and highest TMB (e.g. cutaneous melanoma) exhibited low prevalence of HLA-I LOH while tumors in between exhibited high prevalence of HLA-I LOH. The association of HLA-I LOH with TMB and PD-L1 is also shown in FIG. 8F. Two notable exceptions to the TMB and PD-L1 associations were pancreatic islet cell tumors and adrenocortical carcinomas, both of which had low rates of high TMB (5%-10%) and PD-L1⁺ (3%-7%) despite considerable HLA-I LOH (36%-38%). As shown in FIGS. 9A and 9B, in both diseases, HLA-I LOH was associated with loss of function mutations in DAXX, a tumor suppressor located ˜2 Mb away from HLA-B (both P<0.01). These results suggest that HLA-I LOH in pancreatic islet cell tumors and adrenocortical carcinomas was a passenger event driven by LOH of a nearby tumor suppressor gene. Overall, HLA-I LOH exhibited a linear association with PD-L1⁺ and a complex relationship with high TMB.

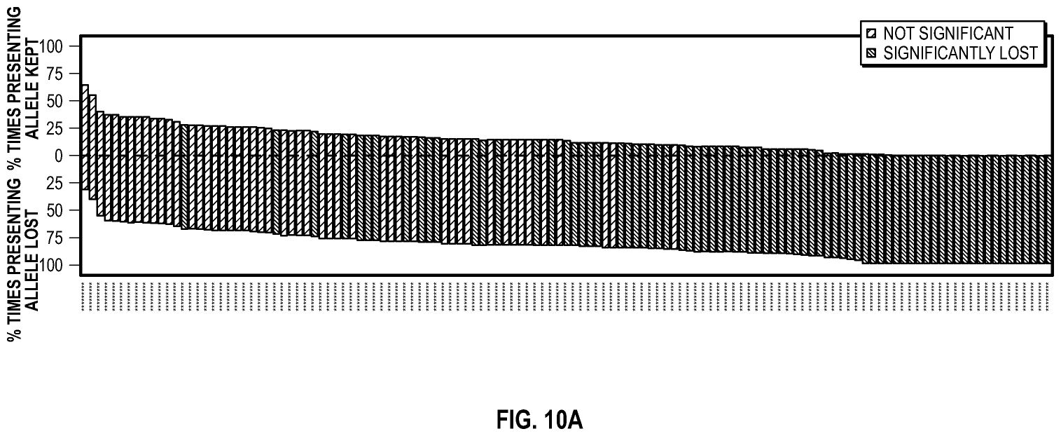

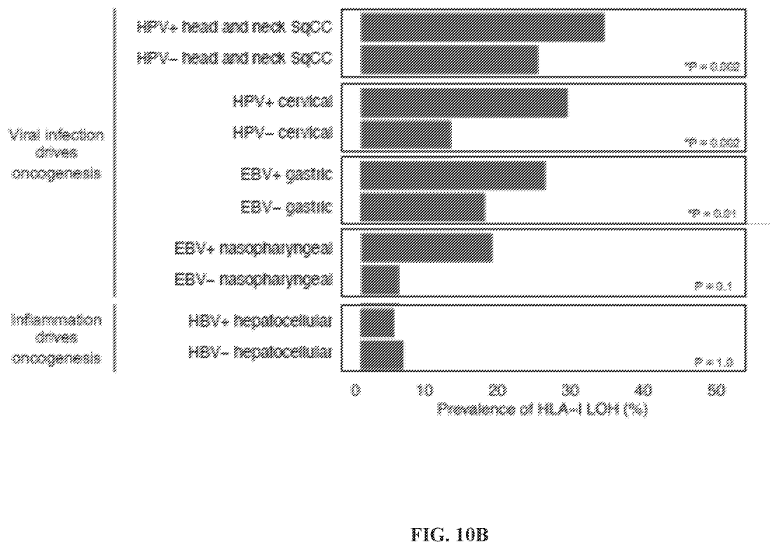

Given the complex relationship between TMB and HLA-I LOH, the link between tumor antigens and HLA-I LOH was further assessed. Neoantigenic driver mutations present a unique subset of neoantigens in that the mutation drives oncogenesis but also provokes an immune response. Recurrent driver neoantigens were predicted using NetMHCpan [14] and cases with HLA-I LOH were assessed for whether the presenting allele was lost or kept. As is shown in FIG. 10A, the presenting allele was more frequently lost for 98% (125/127) of predicted driver neoantigens and 62% (77/125) were statistically significant (P<0.05). Overall, no recurrent driver neoantigen was significantly more frequently presented on the kept allele in any HLA-I LOH event assessed. Viral infection can also drive oncogenesis and recognition by the immune system [15]. FIG. 10B shows the prevalence of HLA-I LOH in virally infected subsets. In tumor types where viral infection mediates cell intrinsic oncogenic transformation, such as with human papillomavirus [16] and Epstein-Barr virus [17], the prevalence of HLA-I LOH was increased in the virally infected subsets (HPV head and neck SqCC: P=0.002, HPV⁺ cervical: P=0.002, EBV⁺ gastric: P=0.01, EBV nasopharyngeal: P=0.1). In contrast, hepatitis B virus, which induces cellular transformation through hepatitis and cirrhosis after chronic infection [18], was not associated with HLA-I LOH in hepatocellular carcinoma (HBV⁺ hepatocellular: P=1.0). These data implicate HLA-I LOH as a potential mechanism by which tumors abrogate neoantigen presentation.

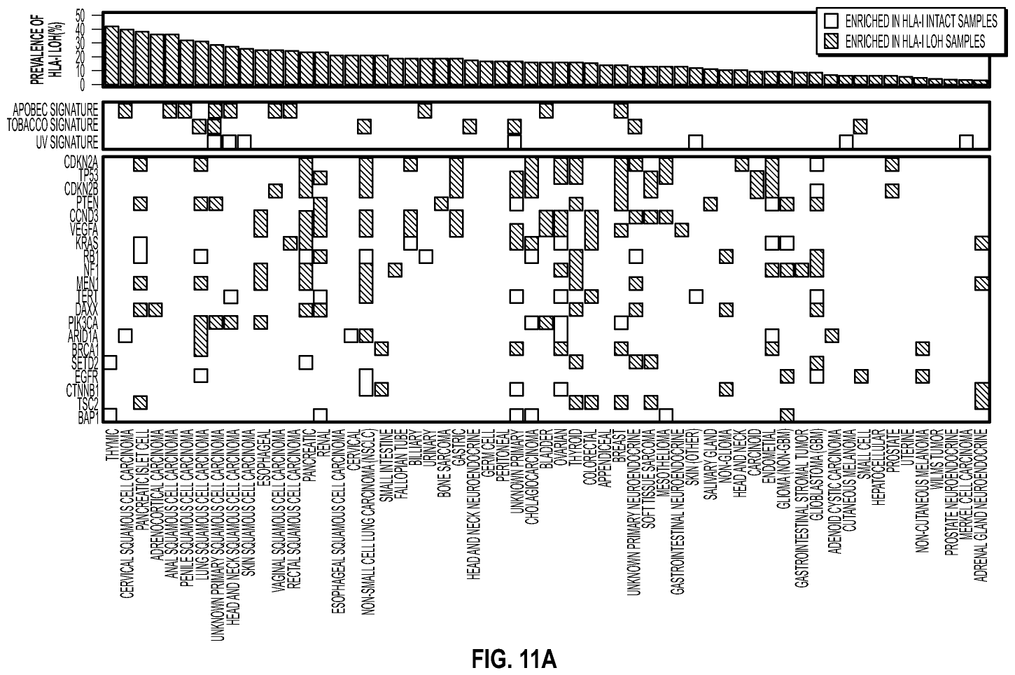

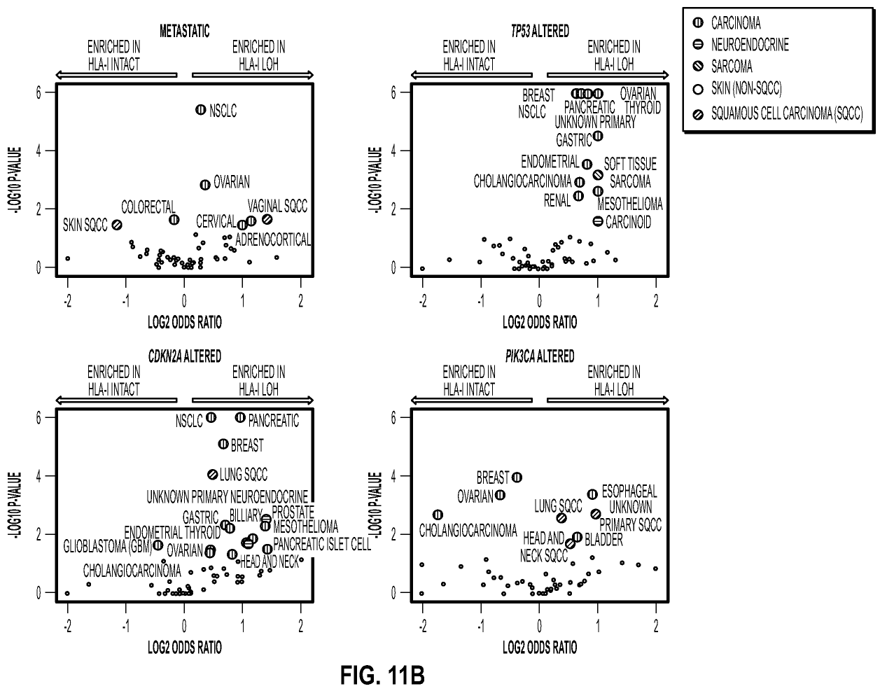

Lastly, the enrichment patterns of frequent genomic alterations, mutational signatures, PD-L1 staining, and TMB status between samples with and without HLA-I LOH across all tumor types was investigated. FIG. 11A depicts the results of this analysis. Tumors with HLA-I LOH were enriched (P<0.05) for high TMB across a diverse range of 15 tumor types, with high TMB being defined as above the median within that disease, as is shown in FIG. 11B. PD-L1⁺ was also largely concurrent with HLA-I LOH, although only reaching statistical significance in four diseases likely due to only a subset having PD-L1 information (FIG. 11B). Several genes were frequently associated with HLA-I LOH including TP53, which was uniformly associated with HLA-I LOH across 14 tumor types, CDKN2A, which had a significant association with HLA-I LOH in 16 diseases, and PIK3CA, which was significantly associated with HLA-I LOH in 5 tumor types, 3 of which were squamous cell carcinomas (FIG. 11B). Gliomas were a notable exception to many of these trends, with mutual exclusivity observed between HLA-I LOH and high TMB as well as CDKN2A alterations, as depicted in FIG. 11A.

From these data, three primary conclusions can be made. The first is that HLA-I LOH is mechanistically connected to presentation of neoantigens. However, utilization of HLA-I LOH as an immune escape mechanism follows a “Goldilocks” pattern, whereby tumors with few neoantigens do not need to lose HLA-I, tumors with high numbers of neoantigens would still present neoantigens after HLA-I LOH, but tumors with an intermediate number of neoantigens can successfully abrogate neoantigen presentation by HLA-I LOH. The second conclusion made from these data is that HLA-I LOH is evolutionarily connected to PD-L1 expression as an immune evasion mechanism, with a clear linear association between HLA-I LOH and PD-L1⁺. And finally, that HLA-I LOH has the potential to refine TMB as a biomarker of checkpoint inhibitor responses based on a better understanding of neoantigen presentation by the tumor.

In non-inflamed tumors, the incidence of HLA-I LOH is low. Thus, high TMB in these tumors may indeed enrich for patients with superior responses to checkpoint inhibitors. This may explain the responses to monotherapy ICI seen in the pan-tumor trial investigating pembrolizumab efficacy in patients representing non-inflamed cancers with high TMB [19]. In contrast, high TMB was not predictive of overall survival in phase II trials of NSCLC. In this example, it was that the TMB high, HLA-I LOH cohort had a similar overall survival to the TMB low, HLA-I intact cohort, suggesting that HLA-I LOH blunted the effect of high TMB in NSCLC. This finding suggests combining HLA-I LOH and TMB would lead to better patient stratification in NSCLC. Furthermore, antigen presentation by the tumor will be an important consideration for designing therapeutic modalities such as neo-antigen vaccines. Assessing HLA-I LOH may play an important role in identifying patients who may be eligible for such therapeutic modalities.

While particular embodiments of the present invention have been shown and described, it will be apparent to those skilled in the art that various changes and modifications in form and details may be made therein without departing from the spirit and scope of the invention as defined by the following claims. The claims that follow are intended to include all such variations and modifications that might fall within their scope, and should be interpreted in the broadest sense allowable by law.

### REFERENCES

1. Reck, M., el al. Pembrolizumab versus Chemotherapy for PD-L1-Positive Non-Small-Cell Lung Cancer. N Engl J Med 375, 1823-1833 (2016).
2. Hellmann, M. D., el al. Nivolumab plus Ipilimumab in Lung Cancer with a High Tumor Mutational Burden. N Engl J Med 378, 2093-2104 (2018).
3. Nghiem, P. T., et al. PD-1 Blockade with Pembrolizumab in Advanced Merkel-Cell Carcinoma. N Engl J Med 374, 2542-2552 (2016).
4. Robert, C., et al. Pembrolizumab versus Ipilimumab in Advanced Melanoma. N Engl J Med 372, 2521-2532 (2015).
5. Le, D. T., et al. PD-1 Blockade in Tumors with Mismatch-Repair Deficiency. N Engl J Med 372, 2509-2520 (2015).
6. Mok, T. S. K., et al. Pembrolizumab versus chemotherapy for previously untreated, PD-L1-expressing, locally advanced or metastatic non-small-cell lung cancer (KEYNOTE-042): a randomised, open-label, controlled, phase 3 trial. Lancet 393, 1819-1830 (2019).
7. Schumacher, T. N. & Schreiber, R. D. Neoantigens in cancer immunotherapy. Science 348, 69-74 (2015).
8. Turajlic, S., et al. Insertion-and-deletion-derived tumour-specific neoantigens and the immunogenic phenotype: a pan-cancer analysis. Lancet Oncol 18, 1009-1021 (2017).
9. Rizvi, N. A., et al. Cancer immunology. Mutational landscape determines sensitivity to PD-1 blockade in non-small cell lung cancer. Science 348, 124-128 (2015).
10. McGranahan, N., et al. Allele-Specific HLA Loss and Immune Escape in Lung Cancer Evolution. Cell 171, 1259-1271 e1211 (2017).
11. Singal, G., et al. Association of Patient Characteristics and Tumor Genomics With Clinical Outcomes Among Patients With Non-Small Cell Lung Cancer Using a Clinicogenomic Database. JAMA 321, 1391-1399 (2019).
12. Davis, A. A. & Patel, V. G. The role of PD-L1 expression as a predictive biomarker: an analysis of all US Food and Drug Administration (FDA) approvals of immune checkpoint inhibitors. J Immunother Cancer 7, 278 (2019).
13. Borghaei, H., et al. Nivolumab versus Docetaxel in Advanced Nonsquamous Non-Small-Cell Lung Cancer. N Engl J Med 373, 1627-1639 (2015).
14. Jurtz, V., et al. NetMHCpan-4.0: Improved Peptide-MHC Class I Interaction Predictions Integrating Eluted Ligand and Peptide Binding Affinity Data. J Immunol 199, 3360-3368 (2017).
15. Tortorella, D., Gewurz, B. E., Furman, M. H., Schust, D. J. & Ploegh, H. L. Viral subversion of the immune system. Annu Rev Immunol 18, 861-926 (2000).
16. Munger, K., et al. Mechanisms of human papillomavirus-induced oncogenesis. J Virol 78, 11451-11460 (2004).
17. Young, L. S. & Murray, P. G. Epstein-Barr virus and oncogenesis: from latent genes to tumours. Oncogene 22, 5108-5121 (2003).
18. Ganem, D. & Prince, A. M. Hepatitis B virus infection—natural history and clinical consequences. N Engl J Med 350, 1118-1129 (2004).
19. Chung, H. C., et al. Efficacy and Safety of Pembrolizumab in Previously Treated Advanced Cervical Cancer: Results From the Phase II KEYNOTE-158 Study. J Clin Oncol 37, 1470-1478 (2019).
20. Frampton, G. M., et al. Development and validation of a clinical cancer genomic profiling test based on massively parallel DNA sequencing. Nat Biotechnol 31, 1023-1031 (2013).
21. Sun, J. X., et al. A computational approach to distinguish somatic vs. germline origin of genomic alterations from deep sequencing of cancer specimens without a matched normal. PLoS Comput Biol 14, e1005965 (2018).
22. Chalmers, Z. R., et al. Analysis of 100,000 human cancer genomes reveals the landscape of tumor mutational burden. Genome Med 9, 34 (2017).
23. Zehir, A., et al. Mutational landscape of metastatic cancer revealed from prospective clinical sequencing of 10,000 patients. Nat Med 23, 703-713 (2017).
24. Zerbino, D. R. & Birney, E. Velvet: algorithms for de novo short read assembly using de Bruijn graphs. Genome Res 18, 821-829 (2008).
25. Camacho, C., et al. BLAST+: architecture and applications. BMC Bioinformatics 10, 421 (2009).
26. Szolek, A., et al. OptiType: precision HLA typing from next-generation sequencing data. Bioinformatics 30, 3310-3316 (2014).
27. Robinson, J., et al. IPD-IMGT/HLA Database. Nucleic Acids Res 48, D948-D955 (2020).
28. Li, H., et al. The Sequence Alignment/Map format and SAMtools. Bioinformatics 25, 2078-2079 (2009).
29. Hartmaier, R J., et al. Genomic analysis of 63,220 tumors reveals insights into tumor uniqueness and targeted cancer immunotherapy strategies. Genome Med 9, 16 (2017).

1. A method of detecting loss-of-heterozygosity (LOH) of a human leukocyte antigen (HLA) gene, comprising:
providing a plurality of nucleic acids obtained from a sample from an individual, wherein the plurality of nucleic acids comprises nucleic acids encoding an HLA gene;
optionally, ligating one or more adaptors onto one or more nucleic acids from the plurality of nucleic acids;
amplifying nucleic acids from the plurality of nucleic acids;
capturing a plurality of nucleic acids corresponding to the HLA gene, wherein the plurality of nucleic acids corresponding to the HLA gene is captured from the amplified nucleic acids by hybridization with a bait molecule;
sequencing, by a sequencer, the captured nucleic acids to obtain a plurality of sequence reads corresponding to the HLA gene;
fitting, by one or more processors, one or more values associated with one or more of the plurality of sequence reads to a model; and
based on the model, detecting LOH of the HLA gene and a relative binding propensity for an HLA allele of the HLA gene.

2. The method of claim 1, wherein LOH of the HLA gene and relative binding propensity for an HLA allele of the HLA gene are detected by:
a) obtaining an observed allele frequency for an HLA allele, wherein observed allele frequency corresponds to frequency of nucleic acid(s) encoding at least a portion of the HLA allele as detected among the plurality of sequence reads corresponding to the HLA gene;
b) obtaining a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles;
c) applying an objective function to measure a difference between the relative binding propensity and the observed allele frequency of the HLA allele;
d) applying an optimization model to minimize the objective function;
e) determining an adjusted allele frequency of the HLA allele based on the optimization model and the observed allele frequency; and
f) determining that LOH has occurred when the adjusted allele frequency of the HLA allele is less than a predetermined threshold.

3. The method of claim 1, further comprising, based at least in part on detection of LOH of the HLA gene, administering an effective amount of a treatment other than an immune checkpoint inhibitor (ICI) to the individual.

4. (canceled)

5. The method of claim 1, further comprising: detecting, or acquiring knowledge of, a high tumor mutational burden (TMB) in the sample.

6. The method of claim 5, further comprising, based at least in part on detection of LOH of the HLA gene and high TMB, administering an effective amount of an immune checkpoint inhibitor (ICI) to the individual.

7. (canceled)

8. The method of claim 1, wherein the HLA gene is a human HLA-A, HLA-B, or HLA-C gene.

9. (canceled)

10. The method of claim 1, wherein the sample comprises tumor cells and/or tumor nucleic acids.

11. The method of claim 10, wherein the sample:
(a) further comprises non-tumor cells;
(b) is from a tumor biopsy or tumor specimen;
(c) comprises tumor cell-free DNA (cfDNA);
(d) comprises fluid, cells, or tissue; or
(e) comprises a tumor biopsy or a circulating tumor cell.

12-18. (canceled)

19. The method of claim 5, wherein the TMB is determined based on a number of non-driver somatic coding mutations per megabase of genome sequenced.

20-35. (canceled)

36. A method for determining allele frequency, comprising:
a) receiving, at one or more processors, an observed allele frequency for an allele of a gene, wherein the observed allele frequency corresponds to frequency of nucleic acid(s) encoding at least a portion of the allele as detected among a plurality of sequence reads corresponding to the gene, wherein the plurality of sequence reads was obtained by sequencing nucleic acids encoding the gene or a portion thereof as captured by hybridization with a bait molecule;
b) receiving, at one or more processors, a relative binding propensity for the allele to the bait molecule, wherein the relative binding propensity of the allele corresponds to propensity of nucleic acid encoding at least a portion of the allele to bind the bait molecule in the presence of nucleic acids encoding portions of one or more other alleles of the gene;
c) executing, by the one or more processors, an objective function to measure a difference between the relative binding propensity and the observed allele frequency of the allele;
d) executing, by the one or more processors, an optimization model to minimize the objective function; and
e) determining, by the one or more processors, an adjusted allele frequency of the allele based on the optimization model and the observed allele frequency.

37. The method of claim 36, wherein the optimization model is a least squares optimization model.

38. The method of claim 36, wherein the optimization model is subject to one or more constraints.

39. The method of claim 38, wherein the one or more constraints require that a median value of the relative binding propensities for a plurality of alleles of the gene is equal to 1.

40. The method of claim 36, wherein the observed allele frequency corresponds to relative frequency of nucleic acid(s) encoding at least a portion of the allele as detected among the plurality of sequence reads, as compared to a reference value.

41-43. (canceled)

44. The method of claim 36, wherein the gene is ST7/RAY1, ARH1/NOEY2, TSLC1, RB, PTEN, SMAD2, SMAD4, DCC, TP53, ATM, miR-15a, miR-16-1, NAT2, BRCA1, BRCA2, hOGG1, CDH1, IGF2, CDKN1C/P57, MEN1, PRKAR1A, H19, KRAS, BAP1, PTCH1, SMO, SUFU, NOTCH1, PPP6C, LATS1, CASP8, PTPN14, ARID1A, FBXW7, M6P/IGF2R, IFN-alpha, an olfactory receptor gene, CBFA2T3, DUTT1, FHIT, APC, P16, FCMD, TSC2, miR-34, c-MPL, RUNX3, DIRAS3, NRAS, miR-9, FAM50B, PLAGL1, ER, FLT3, ZDBF2, GPR1, c-KIT, NAP1L5, GRB10, EGFR, PEG10, BRAF, MEST, JAK2, DAPK1, LIT1, WT1, NF-1, PR, c-CBL, DLK1, AKT1, SNURF, a cytochrome P450 gene (CYP), ZNF587, SOCS1, TIMP2, RUNX1, AR, CEBPA, C19MC, EMP3, ZNF331, CDKN2A, PEG3, NNAT, GNAS, or GATA5.

45. The method of claim 36, further comprising, after determining the adjusted allele frequency: determining that the gene has undergone loss-of-heterozygosity (LOH) based at least in part on the adjusted allele frequency.

46. (canceled)

47. The method of claim 36, further comprising, prior to receiving the observed allele frequency: sequencing a plurality of polynucleotides by next-generation sequencing (NGS), whole exome sequencing, or methylation sequencing in order to obtain the plurality of sequence reads, wherein the plurality of polynucleotides comprises nucleic acid(s) encoding at least a portion of the allele.

48. The method of claim 47, further comprising, prior to sequencing the plurality of polynucleotides:
contacting a mixture of polynucleotides with the bait molecule under conditions suitable for hybridization, wherein the mixture comprises a plurality of polynucleotides capable of hybridization with the bait molecule; and
isolating a plurality of polynucleotides that hybridized with the bait molecule, wherein the isolated plurality of polynucleotides that hybridized with the bait molecule are sequenced.

49-52. (canceled)

53. The method of claim 36, further comprising:
(1) receiving, at one or more processors, an observed allele frequency for each of two or more alleles of a gene, wherein the observed allele frequencies correspond to frequency of nucleic acid(s) encoding at least a portion of the respective allele as detected among a plurality of sequence reads corresponding to the gene, wherein the plurality of sequence reads was obtained by sequencing nucleic acids encoding the gene or a portion thereof as captured by hybridization with a bait molecule;
(2) receiving, at one or more processors, a relative binding propensity for each of two or more alleles to the bait molecule, wherein a second of the two or more alleles has a lower relative binding propensity to the bait molecule than a first of the two or more alleles; and
(3) identifying, by the one or more processors, a second bait molecule, wherein the second of the two or more alleles has a higher relative binding propensity to the second bait molecule than to the first bait molecule.

54. (canceled)

55. A non-transitory computer-readable storage medium comprising one or more programs for execution by one or more processors of a device, the one or more programs including instructions which, when executed by the one or more processors, cause the device to perform the method of claim 36.

56-77. (canceled)

78. A method of treating or delaying progression of cancer, comprising:
(1) detecting loss-of-heterozygosity (LOH) of a human leukocyte antigen (HLA) gene in a sample obtained from an individual, wherein LOH of the HLA gene is detected by:
a) receiving, at one or more processors, an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule;
b) receiving, at one or more processors, a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles;
c) executing, by the one or more processors, an objective function to measure a difference between the relative binding propensity and the observed allele frequency of the HLA allele;
d) executing, by the one or more processors, an optimization model to minimize the objective function;
e) determining, by the one or more processors, an adjusted allele frequency of the HLA allele based on the optimization model and the observed allele frequency; and
f) determining, by the one or more processors, that LOH has occurred when the adjusted allele frequency of the HLA allele is less than a predetermined threshold; and
(2) based at least in part on detection of LOH of the HLA gene, administering an effective amount of a treatment other than an immune checkpoint inhibitor (ICI) to the individual.

79. A method of treating or delaying progression of cancer, comprising:
(1) detecting lack of loss-of-heterozygosity (LOH) of a human leukocyte antigen (HLA) gene in a sample obtained from an individual, wherein lack of LOH of the HLA gene is detected by:
a) receiving, at one or more processors, an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule;
b) receiving, at one or more processors, a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles;
c) executing, by the one or more processors, an objective function to measure a difference between the relative binding propensity and the observed allele frequency of the HLA allele;
d) executing, by the one or more processors, an optimization model to minimize the objective function;
e) determining, by the one or more processors, an adjusted allele frequency of the HLA allele based on the optimization model and the observed allele frequency; and
f) determining, by the one or more processors, that LOH has not occurred when the adjusted allele frequency of the HLA allele is greater than a predetermined threshold; and
(2) based at least in part on detection of lack of LOH of the HLA gene, administering an effective amount of an immune checkpoint inhibitor (ICI) to the individual.

80. The method of claim 79, wherein the ICI comprises a PD-1 inhibitor, a PD-L1 inhibitor, or a CTLA-4 inhibitor.

81. The method of claim 78, wherein the method further comprises detecting a tumor mutation burden (TMB) in a sample obtained from the individual.

82-85. (canceled)

86. The method of claim 81, wherein LOH of the HLA gene and high TMB are detected in the same sample obtained from the individual.

87. The method of claim 81, wherein LOH of the HLA gene and high TMB are detected in different samples obtained from the individual.

88-89. (canceled)

90. A method of treating or delaying progression of cancer, comprising:
(1) detecting loss-of-heterozygosity (LOH) of a human leukocyte antigen (HLA) gene in a sample obtained from an individual, wherein LOH of the HLA gene is detected by:
a) receiving, at one or more processors, an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule;
b) receiving, at one or more processors, a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles;
c) executing, by the one or more processors, an objective function to measure a difference between the relative binding propensity and the observed allele frequency of the HLA allele;
d) executing, by the one or more processors, an optimization model to minimize the objective function;
e) determining, by the one or more processors, an adjusted allele frequency of the HLA allele based on the optimization model and the observed allele frequency; and
f) determining, by the one or more processors, that LOH has occurred when the adjusted allele frequency of the HLA allele is less than a predetermined threshold;
(2) detecting high tumor mutational burden (TMB) in a sample obtained from the individual; and
(3) based at least in part on detection of LOH of the HLA gene and high TMB, administering an effective amount of a treatment comprising an immune checkpoint inhibitor (ICI) to the individual.

91-110. (canceled)

111. A non-transitory computer readable storage medium comprising one or more programs executable by one or more computer processors for performing a method, comprising:
receiving, using the one or more processors, an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule;
receiving, using the one or more processors, a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles;
executing, using the one or more processors, an objective function to measure a difference between the relative binding propensity and the observed allele frequency of the HLA allele;
executing, using the one or more processors, an optimization model to minimize the objective function;
determining, using the one or more processors, an adjusted allele frequency of the HLA allele based on the optimization model and the observed allele frequency; and
determining, using the one or more processors, that LOH has occurred when the adjusted allele frequency of the HLA allele is less than a predetermined threshold.

112-123. (canceled)

124. A system, comprising:
one or more processors; and
a memory configured to store one or more computer program instructions, wherein the one or more computer program instructions when executed by the one or more processors are configured to:
determine an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule;
determine a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles;
execute an objective function to measure a difference between the relative binding propensity and the observed allele frequency of the HLA allele;
execute an optimization model to minimize the objective function;
determine an adjusted allele frequency of the HLA allele based on the optimization model and the observed allele frequency; and
determine that LOH has occurred when the adjusted allele frequency of the HLA allele is less than a predetermined threshold.

125-136. (canceled)
